SEQUENCE LISTING (10 sequences)

SEQ ID NO: 1 - protein, 31 aa, Borrelia burgdorferi
KHLWMHKALLENCDCFCHDENESKCCECDAK

SEQ ID NO: 2 - protein, 27 aa, Borrelia turicatae
KYLWMYRSLLSDCECPCHKKGCCSEKH

SEQ ID NO: 3 - protein, 67 aa, Borrelia miyamotoi
KYLWMYRSLLNNCKCSCDKDDCKCSCDKDDCKCSCDKDDCKCSCDKDDCKCSCDKKEYFSENDDLVI

SEQ ID NO: 4 - protein, 29 aa, Borrelia garinii
KHLWMHKALLENCDCFCHDEHKCCECDIK

SEQ ID NO: 5 - protein, 29 aa, Borrelia afzelii
KYLWMHKALLENCDCVCHDDNKCCECDIK

SEQ ID NO: 6 - protein, 11 aa, Helicobacter pylori
KSIWMLEAHLA

SEQ ID NO: 7 - protein, 12 aa, Treponema pallidum
KAIWMLGATLKA

SEQ ID NO: 8 - protein, 15 aa, Leptospira interrogans
KTAWMLRSLLFVDNT

SEQ ID NO: 9 - protein, 11 aa, Yersinia pestis
KFLWFIESNIE

SEQ ID NO: 10 - protein, 11 aa, Escherichia coli
KFLWFIESNIE

(19) United States
(12) Patent Application Publication
(10) Pub. No.: US 2022/0380443 A1
(43) Pub. Date: Dec. 1, 2022

(54) TARGETING NAPA-LINKED PEPTIDOGLYCAN FOR TREATING LYME DISEASE

(71) Applicant: Virginia Tech Intellectual Properties, Inc., Blacksburg, VA (US)

(72) Inventors: Brandon JUTRAS, Blacksburg, VA (US); Marisela M. DAVIS, Blacksburg, VA (US)

(21) Appl. No.: 17/748,984

(22) Filed: May 19, 2022

Related U.S. Application Data
(60) Provisional application No. 63/190,555, filed on May 19, 2021.

(51) Int. Cl. C07K 16/12

(52) U.S. Cl. CPC C07K 16/1207

(57) ABSTRACT

Described in certain example embodiments herein are methods of treating or preventing a Borrelia burgdorferi (B. burgdorferi) infection, a symptom thereof, or a disease, disorder or condition resulting therefrom in a subject in need thereof that include reducing or eliminating a B. burgdorferi peptidoglycan-associated protein (PAP), optionally neutrophil attracting protein A (NapA), a function thereof, activity thereof, or any combination thereof in the subject in need thereof. Also described herein are methods of diagnosing and/or prognosing B. burgdorferi infection in a subject that include detecting a B. burgdorferi PAP, optionally NapA.

### CROSS-REFERENCE TO RELATED APPLICATIONS

This application claims the benefit of and priority to co-pending U.S. Provisional Patent Application No. 63/190,555, filed on May 19, 2021, entitled “TARGETING NAPA-LINKED PEPTIDOGLYCAN FOR TREATING LYME DISEASE,” the contents of which is incorporated by reference herein in its entirety.

### SEQUENCE LISTING

This application contains a sequence listing filed in electronic form as an ASCII.txt file entitled VTIP-0330US_ST25.txt, created on May 19, 2022 and having a size of 3,113 bytes (4 KB on disk). The content of the sequence listing is incorporated herein in its entirety.

### TECHNICAL FIELD

The subject matter disclosed herein is generally directed to diagnosing and treating Lyme disease or symptoms thereof.

### BACKGROUND

The spirochetal bacterium Borrelia burgdorferi is the primary agent of Lyme disease, a debilitating infection that is transmitted to humans by the bite of an infected Ixodes spp. of tick. Over the past 20 years in the United States, the incidence of Lyme disease has increased more than 2000 percent with an estimate of close to 476,000 patients diagnosed annually. Increases in disease prevalence can be attributed to 1) geographical expansion of vector ticks; 2) higher pathogen carriage rates; 3) deforestation; 4) increase in physician awareness of Lyme disease; and 5) social behavior. Given the number of complex variables contributing to the ascendency of Lyme disease, this pervasive problem is likely to continue for the foreseeable future, which warrants the development of new diagnostics and treatments.

Citation or identification of any document in this application is not an admission that such a document is available as prior art to the present invention.

### SUMMARY

Described in certain example embodiments herein are methods of treating or preventing a Borrelia burgdorferi (B. burgdorferi) infection, a symptom thereof, or a disease, disorder or condition resulting therefrom in a subject in need thereof, the method comprising reducing or eliminating a B. burgdorferi peptidoglycan-associated protein (PAP), a function thereof, activity thereof, or any combination thereof in the subject in need thereof. In certain example embodiments, the B. burgdorferi PAP is neutrophil attracting protein A (NapA).

In certain example embodiments, reducing or eliminating a B. burgdorferi peptidoglycan-associated protein (PAP), a function thereof, activity thereof, or any combination thereof in the subject in need thereof comprises administering an antibody or fragment thereof capable of specifically binding the PAP, optionally NapA protein, or an enzyme capable of targeting, degrading, modifying, and/or otherwise inhibiting the PAP, optionally the NapA protein

In certain example embodiments, the B. burgdorferi peptidoglycan-associated protein (PAP), a function thereof, activity thereof, or any combination thereof is reduced 1-5,000 fold. In certain example embodiments, the disease, disorder, or condition resulting from the B. burgdorferi infection is inflammation, optionally neutrophil mediated-inflammation.

In certain example embodiments, the inflammation is intra-articular inflammation. In certain example embodiments, the disease, disorder or condition resulting from B. burgdorferi infection is arthritis, optionally rheumatoid arthritis, carditis, encephalitis, paralysis, optionally neurological paralysis, a wound, or any combination thereof.

Described in certain example embodiments herein are methods of diagnosing or prognosing a Borrelia burgdorferi (B. burgdorferi) infection, a symptom thereof, or a disease, disorder or condition resulting therefrom in a subject in need thereof, the method comprising detecting a B. burgdorferi peptidoglycan-associated protein (PAP) in a sample obtained from the subject in need thereof. In certain example embodiments, the B. burgdorferi PAP is neutrophil attracting protein A (NapA).

In certain example embodiments, the sample comprises B. burgdorferi outer membrane vesicles. In certain example embodiments, the PAP is present in the B. burgdorferi outer membrane vesicles.

In certain example embodiments, the method further comprises detecting an amount of IL-17 in the sample, wherein an increase in IL-17 as compared to a suitable control indicates B. burgdorferi infection. In certain example embodiments, detecting comprises exposing the sample or component(s) thereof to peripheral blood mononuclear cells in culture and measuring an amount IL-17 in the culture supernatant, whereby an increase in the amount of IL-17 as compared to a suitable control indicates the presence of a PAP. In certain example embodiments, the PAP is neutrophil attracting protein A (NapA).

In certain example embodiments, the sample is a bodily fluid, optionally blood or fraction thereof or synovial fluid.

In certain example embodiments, detecting comprises mass-spectrometry, protein sequencing, an immunodetection method or technique, or any combination thereof.

In certain example embodiments, the method further comprises treating the Borrelia burgdorferi (B. burgdorferi) infection, a symptom thereof, or a disease, disorder or condition resulting therefrom in the subject in need thereof, wherein treating comprises reducing or eliminating a B. burgdorferi peptidoglycan-associated protein (PAP), a function thereof, activity thereof, or any combination thereof in the subject in need thereof. In certain example embodiments, the PAP is neutrophil attracting protein A (NapA).

In certain embodiments, treating comprises administering a PAP, optionally NapA, inhibitor to the subject in need thereof. In certain example embodiments, treating comprises administering to the subject in need thereof comprises administering an antibody or fragment thereof capable of specifically binding the PAP, optionally NapA protein, or an enzyme capable of targeting, degrading, modifying, and/or otherwise inhibiting the PAP, optionally the NapA protein.

In certain example embodiments, the B. burgdorferi peptidoglycan-associated protein (PAP), a function thereof, activity thereof, or any combination thereof is reduced 1-5,000 fold.

Also described in certain example embodiments herein are methods of diagnosing, prognosing, and/or treating B. burgdorferi infection, a symptom thereof, or a disease, disorder or condition resulting therefrom in the subject in need thereof comprising detecting a B. burgdorferi Peptidoglycan reactome signature in peripheral blood mononuclear cells in a sample obtained from a subject.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1G—Identification of peptidoglycan-associated proteins (PAPs) in B. burgdorferi. (FIG. 1A) Peptidoglycan was isolated from live B. burgdorferi (phase-contrast micrograph, scale bar 5 μm) and treated with trypsin (+T). Peptides from each preparation were identified by LC-MS. (FIG. 1B) Western blot analysis of whole cell lysates prepared from the parental, wild-type strain (5A11) and napA mutant (5A11/napA). Each preparation was assayed by western blot for NapA (left) and the constitutive protein FlaB (right). The latter served as a loading control. Numbers and dashes correspond to the migration of molecular weight standards. (FIG. 1C-1E) Localization of putative PAP NapA by sub-cellular fractionation coupled with immunofluorescence. Both 5A11 and 5A11/napA were transformed with a B. burgdorferi shuttle vector constitutively expressing GFP (GFP, purple panel). Each strain was fixed and treated with sodium phosphate buffer (no permeabilization, FIG. 1C); 50% methanol (outer membrane permeabilization, (FIG. 1D); or methanol, followed by SDS and lysozyme (outer/inner membrane permeabilization, (FIG. 1E). Both strains, treated with each permeabilization method, were probed for three targets, independently: Anti-FlaB (periplasmic control, green), anti-GFP (cytoplasmic control, green), and anti-NapA (green). Secondary antibodies anti-Rat IgG:Alexa 588 (anti-FlaB) and anti-Rabbit IgG:Alexa 647 (anti-GFP/anti-NapA) were used to detect primary antibodies. In all cases, images were acquired by phase-contrast microscopy (Ph), epifluorescence microscopy (middle two panels), and epifluorescence channels were merged (M). All scale bars=5 μm. (FIG. 1F) Population level analysis of signal intensities from each treatment in FIG. 1C-1E. Phase-contrast micrographs were used for automated cell detection, and total signal intensities, for each cell, were calculated and used to generate violin plots. No permeabilization (upper panel); outer membrane permeabilization (middle panel); outer/inner membrane permeabilization (lower panel) are shown and grouped by strain, and target. Each data set contained>300 cells. All average signal intensities were statistically significant (unpaired t-test, p<0.001) between upper panel and lower two panels, except for anti-NapA in 5A11/napA strain. (FIG. 1G) Demographs of NapA signal attained from outer membrane permeabilization (periplasmic signal, upper panel) and outer/inner membrane permeabilization (cytoplasmic signal, lower panel). Cells were organized by cell length, fluorescent intensity profiles were generated for each cell, and plotted as a heatmap (0-1).

FIG. 2A-2E—NapA is a PAP. (FIG. 2A) Dot blot analysis of PG. Wild-type (5A11) and napA mutant (5A11/napA) bacteria were cultured to mid-log exponential growth, cells were harvested, and PG was purified. Prior to trypsin treatment, one half of each sample was removed. Serial dilutions of each pre- and post-trypsin preparation were spotted on nitrocellulose and probed for PG (anti-PG, left) or NapA (anti-NapA, right). (FIG. 2B) The same sample preparation described in A were used for immunofluorescence studies. Whole PG sacculi were visualized by epifluorescence microscopy using wheat germ agglutinin (WGA, red) conjugated to Alexa Fluor 350. NapA (green) was detected using anti-NapA antibody and anti-rabbit IgG conjugated to Alexa Fluor 488. Scale bars=5 μm. (FIG. 2C) Population-level analysis of integrated fluorescent signal intensities of NapA from sacculi isolated from 5A11 (n=310) and 5A11/napA (n=345). (FIG. 2D) Scatter-plot analysis of NapA signal in methanol treated, fixed cells (gray, n=532 see FIG. 1D) relative to NapA signal from purified PG (red, n=310), pre-trypsin treatment. Scatter-plot shading corresponds to +/−1 standard deviation (STD) while dark lines represent moving averages. (FIG. 2E) Proteinase K assay to determine protein surface exposure. Both 5A11 and 5A11/napA were cultured to identical densities, each split in half, gently harvested by centrifugation, and treated with (+) 5 ug/mL of Proteinase K (ProK) or PBS diluent control (−). After 1 hour protease was inactivated and surface exposure of FlaB (left), OspA (middle), or NapA (right) was determined by western blot.

FIG. 3A-3D—Cell envelope stress and defects of NapA deficient bacteria. (FIG. 3A) Osmotic and lysozyme susceptibility in wild-type (5A11) and napA mutant (5A11/napA) bacteria. Following exposure to 0.100 M NaCl (total osmolality 544 mOsm, left) or 0.375 mg/mL Lysozyme (right) for 18 hours, each strain was diluted in fresh media and plated. Three (wild-type) or six (mutant) weeks later, CFUs were determined. Bars shown are the mean+/−standard deviation from 4 experimental BSK II plates with either strain. P-value determined using unpaired t-test, *=p<0.05. (FIG. 3B) Cryo-electron micrographs of the inner membrane (IM), peptidoglycan (PG) and outer membrane (OM) of the 5A11 (top) and 5A11/napA (bottom) strains. Scale bar 100 nm. (FIG. 3C) Population-level analysis of average PG width (right) and average PG pixel intensity (left) normalized by sampling area. Note that measurements excluded PG from 10% of each cell pole since these areas are thicker and more variable. (FIG. 3D) Liquid chromatography spectra attained from muropeptides, isolated from strain 5A11 (black) and 5A11/napA (red). Each strain was cultured, enumerated, and PG was purified for an equal number of bacteria. Following mutanolysin digestion, an equal amount of each sample was injected, and muropeptide abundance (UV absorbance) was plotted as a function of retention time.

FIG. 4—NapA is released in outer-membrane vesicles. (A) Wild-type (5A11) and napA mutant (5A11/napA) were cultured to mid-log (2.5×107 cell/mL), cells were collected, washed, and processed into crude lysate (L), Outer membrane vesicles (OMVs), or protoplasmic cylinders (PC) as described in the methods. Each fraction was standardized by total amount of protein (Bradford assay) and assayed by immunoblot for OspA (anti-OspA, upper panel), FlaB (anti-FlaB, middle panel), or NapA (anti-NapA, lower panel).

FIG. 5A-5B—NapA-PG is released in outer-membrane vesicles. (FIG. 5A) The same OMV and PC fractions analyzed in FIG. 4 were serially diluted and assayed for NapA and PG by dot blot. (FIG. 5B) Reporter assay to query PC and OMV fractions for PG containing Muramyl dipeptide (MDP). Human NOD2 reporter cell line (hNOD2, Invivogen) was used to estimate the relative amount of MDP in sample with and without 20 ug/mL of gefitinib—an inhibitor of the effector downstream of hNOD2, RIP2. MDP (50 pg/mL) served as the positive control (C) reactions. Bars shown are the mean of samples tested in triplicate, +/−standard deviation. **=p<0.001, unpaired t-test, with and without inhibitor.

FIG. 6A-6C—NapA stimulates IL-17 and induces neutrophil migration. (FIG. 6A) IL-17 production by human peripheral blood mononuclear cells (PBMCs). Three pools of eight mixed donor PBMCs samples were stimulated with 10 ug/mL of PG, before and after trypsin treatment, from wild-type and napA mutant bacteria. Culture supernatants, from each stimulation, were assayed for IL-17 by ELISA (Abcam). Values are the mean, +/−standard deviation, after normalizing for untreated, PBS diluent control are shown. Statistical analysis unpaired t-test, *=p<0.005. (FIG. 6B) Merged Phase-contract/epifluorescence micrograph of microfluidic competitive chemotaxis-chip (μC3) (55) used to measure dHL-60 cell (blue) migration both toward (red) and away (black) from gradients of each stimulus. Scale bar=500 (FIG. 6C) dHL-60 cells show a higher percentage of cells migrating toward PG-linked NapA. Reservoirs that flank each maze were loaded with 125 μg/mL of each PG sample, diluted in dHL-60 cell culture media, and compared to opposite reservoir, which contained culture media alone. Controls included media, 10 nM of Formylmethionine-leucyl-phenylalanine (fMLP), and 100 nM of Leukotriene B4 (LTB4). Data were collected over 5 hours, images captured every 2 minutes, while cells were maintained at 37° C. under 5% CO2. Results shown are mean+/−SD of three biological replicate experiments. To evaluate differences between responses ANOVA were performed with Turkey's correction for multiple comparisons (*=p<0.05, **=p<0.005).

FIG. 7A-7B—(FIG. 7A) Lysozyme and NaCl stress test. Both 5A11 and 5A11/napA strains were grown to 1×10⁴ cells/mL in BSK II at 37 degrees C. media prior to adding increasing amounts of Lysozyme (0 to 2 mg/mL) (left) or NaCl (7.8 to 500 mM) (right). Final osmolality of culture media is also shown (380 to 1410 mOsm). Cells were allowed to grow for one week in a 96 well plate prior to growth analysis using spectrophotometry. (FIG. 7B) Growth curves. 5A11 and 5A11/napA were grown at a starting concentration of 1×10³ cells/mL in BSK II media. Cells were enumerated roughly every 24 hours for 10 days with the exception of the first count which occurred 48 hours after inoculation. Note that for data presented in FIG. 3A-3D the concentrations of Lysozyme and NaCl tested were in between wells 5-6 and 4-5, respectively.

FIG. 8A-8B—(FIG. 8A) SDS PAGE and immunoblot analysis of Outer Membrane Vesicle and Protoplasmic Cylinder preparations. Both 5A11 and 5A11/napA strains were cultured to late-log, cell were harvested, and fractionated into outer membrane vesicles (OMV) and protoplasmic cyl-inders (PC). Each preparation was separate by SDS PAGE and visualized by Sypro Ruby stain. Asterisk (*) indicate bands only present in OMVs. (FIG. 8B) NapA Immunoblot of samples prepared as described above.

FIG. 9A-9B—dHL60 cells migrated toward NapA-associated PG shows less non-dysfunctional migratory patterns in comparison to other preparations. (FIG. 9A) Cells migrating toward NapA-associated PG shows lowest number of cells displaying of non-directional migration (n=23). (FIG. 9B) Cells migrating toward NapA-associated PG shows lowest number of cells showing oscillatory migration (n=13).

FIG. 10A-10B—dHL-60 cells migrating toward Nap-A associated PG show higher velocity in com-parison to other preparations. Single cell velocity values are plotted over a box plot showing range of values. (FIG. 10A) Cells migrating toward PG bait samples and chemoattractants show Nap-A associated PG has a similar velocity (10.94±4.79 μm/min) to known chemoattractants LTB4 (7.04±4.90 μm/min) and fMLP (6.93±4.40 μm/min). (FIG. 10B) Cells migrating away from PG bait samples and chemoattractants show similar velocities. To evaluate differences between responses ANOVA were performed with Turkey's correction for multiple comparisons (*=p<0.05, ***=p<0.001).

FIG. 11A-11C—Phylogenetic analysis of Dps/NapA. (FIG. 11A) Phylogenic analysis of Dps/NapA homo-logues in Borreliae, Helicobacter pylori, Treponema pallidum, Leptospira interrogans, Yersinia pestis, and Escherichia coli. (FIG. 11B) Amino acid alignment of Dps/NapA homologues from bacteria in A. The Lysine-rich DNA binding domain is underlined (blue) (FIG. 11C) Zoomed in amino acid sequence of the C-terminus of Dps/NapA homologues.

FIG. 12—RNA sequencing sample names.

FIG. 13—Differentially expressed genes of interest in PBMCs stimulated with PG^{Bb} for 12 hours.

FIG. 14—Differentially expressed genes of interest in PBMCs stimulated with PG^{Bb} for 72 hours.

FIG. 15—Differentially expressed genes of interest in PBMCs stimulated with live Borrelia for 12 hours.

FIG. 16—Differentially expressed genes of interest in PBMCs stimulated with live Borrelia for 72 hours.

FIG. 17—Reactome analysis post 12 hour PBMC stimulation with PG^{Sm}.

FIG. 18—Reactome analysis post 12 hour PBMC stimulation with PG^{Sa}.

FIG. 19—Reactome analysis post 12 hour PBMC stimulation with PG^{Ec}.

FIG. 20—Reactome analysis post 12 hour PBMC stimulation with PG^{Bs}.

FIG. 21—Reactome analysis post 12 hour PBMC stimulation with PG^{Bb}.

FIG. 22—Reactome analysis post 72 hour PBMC stimulation with PG^{Sm}.

FIG. 23—Reactome analysis post 72 hour PBMC stimulation with PG^{Sa}.

FIG. 24—Reactome analysis post 72 hour PBMC stimulation with PG^{Ec}.

FIG. 25—Reactome analysis post 72 hour PBMC stimulation with PG^{Bs}.

FIG. 26—Reactome analysis post 72 hour PBMC stimulation with PG^{Bb}.

FIG. 27—GO analysis “Granulocyte Activation” Pathway

FIG. 28—Reactome analysis post 12 hour PBMC stimulation with live Borrelia.

FIG. 29—Reactome analysis post 72 hour PBMC stimulation with live Borrelia.

FIG. 30—DO Enrichment Analysis.

FIG. 31A-31C—Coexpression analysis. (FIG. 31A) Coexpression analysis for PBMCs stimulated with PG^{Bb}, PG^{Ec}, PG^{Bs}, PG^{Sm}, and PG^{Sa} for 12 hours. (FIG. 312) Coexpression analysis for PBMCs stimulated with PG^{Bb}, PG^{Ec}, PG^{Bs}, PG^{Sm}, and PG^{Sa} for 72 hours. (FIG. 31C) PBMCs stimulated with live Borrelia at either 500 cells/mL (Bb1X) or 5000 cells/mL (Bb2X) for 12 or 72 hours.

FIG. 32A-32B—GO Enrichment analysis results for the PMCs stimulated with bacterial PGs for 12 or 72 hours. Top 20 GO results are reported, those with a padj value of <0.05 represent significant findings. (FIG. 32A) GO analysis or PBMs stiulated with bacterial PG for 12 hours. (FIG. 32B) GO analysis for PBMCs stimulated with bacterial PG for 72 hours.

FIG. 33A-33B—Gene expression analysis in PBMCs stimulated with Bacterial PGs for 12 or 72 hours. Y axis is log₂Fold change in gene expression compared to controls. Controls were PBMC stimulated with PBS. (FIG. 33A) Genes upregulated in response to PG^{Bb}, PG^{Ec}, PG^{Bs}, PG^{Sm}, and PG^{Sa} for 12 hours. (FIG. 33B) Genes upregulated in response to PG^{Bb}, PG^{Ec}, PG^{Bs}, PG^{Sm}, and PG^{Sa} for 72 hours.

FIG. 34A-34B—KEGG enrichment analysis results for the PBMCs Stimulated with Bacterial PGs for 12 and 72 hours. Top 20 KEGG results are reported, those with a padj value of <0.05 represent significant findings. (FIG. 34A) KEGG analysis for PBMCs stimulated with bacterial PG for 12 hours. (FIG. 34B) KEGG analysis for PBMCs stimulated with bacterial PG for 72 hours.

FIG. 35—Cluster analysis of differentially expressed genes. Cluster analysis was performed using log 10 (FPKM+1). Red, as represented in greyscale represents genes with high levels of expression. Blue, as represented in greyscale represents genes with low levels of expression. LB1x: live Borrelia 500 cells/mL. LB2x: Live Borrelia 5000 cells/mL.

FIG. 36A-36B—GO Enrichment analysis results for the PBMCs stimulated with PG^{Bb} or live Borrelia at 5000 cells/mL at 12 and 72 hours. Top 20 GO results are reported, those with a padj value of <0.05 represent significant findings. (FIG. 36A) GO analysis for PBMCs stimulated with bacterial PG for 12 hours. (FIG. 36B) GO analysis for PBMCs stimulated with bacterial PG for 72 hours.

FIG. 37A-37B—KEGG enrichment analysis results for the PBMCs stimulated with PG^{Bb} or live Borrelia at 5000 cells/mL at 12 and 72 hours. Top 20 KEGG results are reported, those with a padj value of <0.05 represent significant findings. (FIG. 37A) KEGG analysis for PBMCs stimulated with bacterial PG for 12 hours. (FIG. 34B) KEGG analysis for PBMCs stimulated with bacterial PG for 72 hours.

FIG. 38-38B—Gene expression analysis in PBMCs stimulated with bacterial PGs for 12 or 72 hours. Y-axis is log₂Fold change in gene expression compared to controls (PBMC stimulated with PBS). (FIG. 38A) Genes significantly downregulated in response to PG^{Bb}, PG^{Ec}, PG^{Bs}, PG^{Sm}, and PG^{Sa} for 12 hours. (FIG. 33B) Genes downregulated in response to PG^{Bb}, PG^{Ec}, PG^{Bs}, PG^{Sm}, and PG^{Sa} for 72 hours.

FIG. 39—Gene expression analysis in PBMCs stimulated with Live Borrelia at 5000 cells/mL for 12 and 72 hours. log 2Fold change in gene expression compared to controls (PBMC stimulated with PBS). Genes listed are those of interest relative to those up/down regulated compared to PBMCs stimulated with PG, and also those that were uniquely expressed by cells stimulated with live Borrelia.

The figures herein are for illustrative purposes only and are not necessarily drawn to scale.

### DETAILED DESCRIPTION OF THE EXAMPLE EMBODIMENTS

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant application should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Where a range is expressed, a further aspect includes from the one particular value and/or to the other particular value. Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2nd edition (2011).

Definitions of common terms and techniques in chemistry and organic chemistry can be found in Smith. Organic Synthesis, published by Academic Press. 2016; Tinoco et al. Physical Chemistry, 5^{th} edition (2013) published by Pearson; Brown et al., Chemistry, The Central Science 14^{th} ed. (2017), published by Pearson, Clayden et al., Organic Chemistry, 2^{nd} ed. 2012, published by Oxford University Press; Carey and Sunberg, Advanced Organic Chemistry, Part A: Structure and Mechanisms, 5^{th} ed. 2008, published by Springer; Carey and Sunberg, Advanced Organic Chemistry, Part B: Reactions and Synthesis, 5^{th} ed. 2010, published by Springer, and Vollhardt and Schore, Organic Chemistry, Structure and Function; 8^{th} ed. (2018) published by W.H. Freeman.

Definitions of common terms, analysis, and techniques in genetics can be found in e.g., Hartl and Clark. Principles of Population Genetics. 4^{th} Ed. 2006, published by Oxford University Press. Published by Booker. Genetics: Analysis and Principles, 7th Ed. 2021, published by McGraw Hill; Isik et al., Genetic Data Analysis for Plant and Animal Breeding. First ed. 2017. published by Springer International Publishing AG; Green, E. L. Genetics and Probability in Animal Breeding Experiments. 2014, published by Palgrave; Bourdon, R. M. Understanding Animal Breeding. 2000 2^{nd} Ed. published by Prentice Hall; Pal and Chakravarty. Genetics and Breeding for Disease Resistance of Livestock. First Ed. 2019, published by Academic Press; Fasso, D. Classification of Genetic Variance in Animals. First Ed. 2015, published by Callisto Reference; Megahed, M. Handbook of Animal Breeding and Genetics, 2013, published by Omniscriptum Gmbh & Co. Kg., LAP Lambert Academic Publishing; Reece. Analysis of Genes and Genomes. 2004, published by John Wiley & Sons. Inc; Deonier et al., Computational Genome Analysis. 5^{th} Ed. 2005, published by Springer-Verlag, New York; Meneely, P. Genetic Analysis: Genes, Genomes, and Networks in Eukaryotes. 3^{rd} Ed. 2020, published by Oxford University Press.

As used herein, the singular forms “a”, “an”, and “the” include both singular and plural referents unless the context clearly dictates otherwise.

As used herein, “about,” “approximately,” “substantially,” and the like, when used in connection with a measurable variable such as a parameter, an amount, a temporal duration, and the like, are meant to encompass variations of and from the specified value including those within experimental error (which can be determined by e.g. given data set, art accepted standard, and/or with e.g. a given confidence interval (e.g. 90%, 95%, or more confidence interval from the mean), such as variations of +/−10% or less, +/−5% or less, +/−1% or less, and +/−0.1% or less of and from the specified value, insofar such variations are appropriate to perform in the disclosed invention. As used herein, the terms “about,” “approximate,” “at or about,” and “substantially” can mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

As used herein, a “biological sample” refers to a sample obtained from, made by, secreted by, excreted by, or otherwise containing part of or from a biologic entity. A biologic sample can contain whole cells and/or live cells and/or cell debris, and/or cell products, and/or virus particles. The biological sample can contain (or be derived from) a “bodily fluid”. The biological sample can be obtained from an environment (e.g., water source, soil, air, and the like). Such samples are also referred to herein as environmental samples. As used herein “bodily fluid” refers to any non-solid excretion, secretion, or other fluid present in an organism and includes, without limitation unless otherwise specified or is apparent from the description herein, amniotic fluid, aqueous humor, vitreous humor, bile, blood or component thereof (e.g. plasma, serum, etc.), breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from an organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

All publications, published patent documents, and patent applications cited herein are hereby incorporated by reference to the same extent as though each individual publication, published patent document, or patent application was specifically and individually indicated as being incorporated by reference.

### Overview

The spirochetal bacterium Borrelia burgdorferi is the primary agent of Lyme disease, a debilitating infection that is transmitted to humans by the bite of an infected Ixodes spp. of tick. Over the past 20 years in the United States, the incidence of Lyme disease has increased more than 2000 percent with an estimate of close to 476,000 patients diagnosed annually. Increases in disease prevalence can be attributed to 1) geographical expansion of vector ticks; 2) higher pathogen carriage rates; 3) deforestation; 4) increase in physician awareness of Lyme disease; and 5) social behavior. Given the number of complex variables contributing to the ascendency of Lyme disease, this pervasive problem is likely to continue for the foreseeable future. As such there exists a need for the development of new diagnostics and treatment for Lyme disease and its causative agent Borrelia burgdorferi (B. burgdorferi).

Relative to classical diderms, the B. burgdorferi cell envelope is riddled with anomalies. For example, despite being a diderm, B. burgdorferi does not produce Lipopolysaccharide. The outer membrane (OM) contains host-derived cholesterol and more than 100 different lipoproteins. Flagella are not extruded from the envelope, but rather are contained entirely in the periplasmic space. Cross-linking peptides in the PG cell-wall contain the atypical diamine L-Ornithine. Further, the typical proteins which are associated with PG that provide both structural integrity and spatial continuity within the cell envelope, appear to be lacking. As described and demonstrated herein, Applicants have identified a B. burgdorferi PAP, e.g., neutrophil attracting protein A (NapA), previously implicated as an immunomodulatory factor and determine its function in the cell envelope homeostasis. Further, Applicant has evidenced a PG-PAP relationship that likely contributes to the pathogenic properties of B. burgdorferi PG, particularly when secreted in cell membrane vesicles that are released into a subject during infection.

With that said, embodiments disclosed herein can provide methods of treating or preventing Borrelia burgdorferi (B. burgdorferi) infection, a symptom thereof, or a disease, disorder or condition resulting therefrom in a subject in need thereof that includes reducing or eliminating a B. burgdorferi peptidoglycan-associated protein (PAP), a function thereof, activity thereof, or any combination thereof in the subject in need thereof. In certain example embodiments, the B. burgdorferi PAP is neutrophil attracting protein A (NapA). Described in certain example embodiments herein are methods diagnosing or prognosing a Borrelia burgdorferi (B. burgdorferi) infection, a symptom thereof, or a disease, disorder or condition resulting therefrom in a subject in need thereof, the method comprising detecting a B. burgdorferi peptidoglycan-associated protein (PAP) in a sample obtained from the subject in need thereof. In certain example embodiments, the B. burgdorferi PAP is neutrophil attracting protein A (NapA). Also described in certain example embodiments herein are methods of diagnosing, prognosing, and/or treating B. burgdorferi infection, a symptom thereof, or a disease, disorder or condition resulting therefrom in the subject in need thereof that include detecting a B. burgdorferi Peptidoglycan signature in peripheral blood mononuclear cells in a sample obtained from a subject.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Methods of Treating or Preventing B. Burgdorferi Infection

Described in certain example embodiments herein are methods of treating or preventing a Borrelia burgdorferi (B. burgdorferi) infection, a symptom thereof, or a disease, disorder or condition resulting therefrom in a subject in need thereof, the method comprising reducing or eliminating a B. burgdorferi peptidoglycan-associated protein (PAP), a function thereof, activity thereof, or any combination thereof in the subject in need thereof. In certain example embodiments, the B. burgdorferi PAP is neutrophil attracting protein A (NapA).

In certain example embodiments, reducing or eliminating a B. burgdorferi peptidoglycan-associated protein (PAP), a function thereof, activity thereof, or any combination thereof in the subject in need thereof comprises administering a PAP, optionally a NapA, inhibitor to the subject in need thereof. In some embodiments, the PAP, optionally NapA, inhibitor is an antibody or fragment thereof capable of specifically binding the PAP, optionally NapA protein, or an enzyme capable of targeting, degrading, modifying, and/or otherwise inhibiting the PAP, optionally the NapA protein. In some embodiments, reducing or eliminating a B. burgdorferi PAP, optionally NapA, includes administering an antibody or fragment thereof capable of specifically binding the PAP, optionally NapA protein, or an enzyme capable of targeting, degrading, modifying, and/or otherwise inhibiting the PAP, optionally the NapA protein, to the subject in need thereof.

In some the agent administered effective to reduce or eliminate a B. burgdorferi peptidoglycan-associated protein (PAP) (e.g., NapA), a function thereof, activity thereof, or any combination thereof in the subject in need thereof is a chemical agent, such as a small molecule therapeutic agent, a biologic agent (e.g., protein or nucleic acid, or complex thereof), or any combination thereof. In some embodiments, the agent administered effective to reduce or eliminate a B. burgdorferi peptidoglycan-associated protein (PAP), a function thereof, activity thereof, or any combination thereof in the subject in need thereof is an antibody or fragment thereof that can specifically bind the B. burgdorferi PAP, such as NapA. As used herein, “antibody” refers to a protein or glycoprotein containing at least two heavy (H) chains and two light (L) chains inter-connected by disulfide bonds, or an antigen binding portion thereof. Each heavy chain is comprised of a heavy chain variable region (abbreviated herein as VH) and a heavy chain constant region. Each light chain is comprised of a light chain variable region and a light chain constant region. The VH and VL regions retain the binding specificity to the antigen and can be further subdivided into regions of hypervariability, termed complementarity determining regions (CDR). The CDRs are interspersed with regions that are more conserved, termed framework regions (FR). Each VH and VL is composed of three CDRs and four framework regions, arranged from amino-terminus to carboxy-terminus in the following order: FR1, CDR1, FR2, CDR2, FR3, CDR3, and FR4. The variable regions of the heavy and light chains contain a binding domain that interacts with an antigen. “Antibody” includes single valent, bivalent and multivalent antibodies.

As used herein, “administering” refers to any suitable administration for the agent(s) being delivered and/or subject receiving said agent(s) and can be oral, topical, intravenous, subcutaneous, transcutaneous, transdermal, intramuscular, intra joint, parenteral, intra-arteriole, intradermal, intraventricular, intraosseous, intraocular, intracranial, intraperitoneal, intralesional, intranasal, intracardiac, intraarticular, intracavernous, intrathecal, intravireal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration routes can be, for instance, auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratym panic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated, subject being treated, and/or agent(s) being administered.

In some embodiments, the effective agent is provided as a pharmaceutical formulation. In some embodiments, administration can be one or more times daily, weekly, monthly or yearly.

Methods of identifying agents capable of reducing or eliminating a B. burgdorferi peptidoglycan-associated protein (PAP), such as NapA, a function thereof, activity thereof, or any combination thereof in the subject in need thereof include (a) applying a candidate or test agent to a B. burgdorferi organism or portion thereof, a cell membrane vesicle produced from B. burgdorferi, a cell or cell population obtained from a subject or that contains or expresses a B. burgdorferi PAP, such as NapA, a PAP, such as NapA, or any combination thereof; (b) detecting and/or quantifying an amount, function, and/or activity of the PAP, such as NapA, whereby a reduction and/or elimination of the amount, function, and/or activity of the PAP, such as NapA detected and/or quantified as compared to a suitable control indicates that such a candidate agent is effective to reduce or eliminate a B. burgdorferi peptidoglycan-associated protein (PAP) (such as NapA), a function thereof, and/or activity thereof. As used herein, “agent” refers to any substance, compound, molecule, and the like, which can be administered to a subject on a subject to which it is administered to. An agent can be inert. An agent can be an active agent. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed.

In certain example embodiments, the B. burgdorferi peptidoglycan-associated protein (PAP), a function thereof, activity thereof, or any combination thereof is reduced 1-5,000 fold as compared to a suitable control. In some embodiments, the B. burgdorferi peptidoglycan-associated protein (PAP), a function thereof, activity thereof, or any combination thereof is reduced 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, 5000 fold or more as compared to a suitable control.

In certain example embodiments, the disease, disorder, or condition resulting from the B. burgdorferi infection is inflammation, optionally neutrophil mediated-inflammation. In certain example embodiments, the inflammation is intra-articular inflammation. In certain example embodiments, the disease, disorder or condition resulting from B. burgdorferi infection is arthritis, optionally rheumatoid arthritis, carditis, encephalitis, paralysis (including but not limited to neurological paralysis), a wound(s) (acute and chronic wounds).

### Pharmaceutical Formulations

Also described herein are pharmaceutical formulations that can contain an amount, effective amount, and/or least effective amount, and/or therapeutically effective amount of one or more compounds, molecules, compositions, or a combination thereof (which are also referred to as the primary active agent or ingredient elsewhere herein) effective to reduce or eliminate a B. burgdorferi PAP (e.g., NapA), a function thereof, and/or activity thereof as described in greater detail elsewhere herein and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a B. burgdorferi infection and/or symptom thereof and/or disease, disorder, or condition resulting from the B. burgdorferi infection. In some embodiments, the disease, disorder, or condition resulting from the B. burgdorferi infection is inflammation, optionally neutrophil mediated-inflammation. In certain example embodiments, the inflammation is intra-articular inflammation. In certain example embodiments, the disease, disorder or condition resulting from B. burgdorferi infection is arthritis, optionally rheumatoid arthritis, carditis, encephalitis, paralysis (including but not limited to neurological paralysis), a wound(s) (acute and chronic wounds).

As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g. polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-hi stamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the one or more therapeutic effects are reduction of one or more symptoms or pathologies of a B. burgdorferi infection and/or symptom thereof and/or disease, disorder, or condition resulting from the B. burgdorferi infection. In some embodiments, the disease, disorder, or condition resulting from the B. burgdorferi infection is inflammation, optionally neutrophil mediated-inflammation. In certain example embodiments, the inflammation is intra-articular inflammation. In certain example embodiments, the disease, disorder or condition resulting from B. burgdorferi infection is arthritis, optionally rheumatoid arthritis, carditis, encephalitis, paralysis (including but not limited to neurological paralysis), a wound(s) (acute and chronic wounds). In some embodiments, the one or more therapeutic effects are reduction of one or more symptoms or pathologies of Lyme's disease.

The effective amount, least effective amount, and/or therapeutically effective amount of the primary and optional secondary active agent described elsewhere herein contained in the pharmaceutical formulation can be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, mg, or g or be any numerical value or subrange within any of these ranges.

In some embodiments, the effective amount, least effective amount, and/or therapeutically effective amount can be an effective concentration, least effective concentration, and/or therapeutically effective concentration, which can each be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value or subrange within any of these ranges.

In other embodiments, the effective amount, least effective amount, and/or therapeutically effective amount of the primary and optional secondary active agent be any non-zero amount ranging from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value or subrange within any of these ranges.

In some embodiments, the primary and/or the optional secondary active agent present in the pharmaceutical formulation can be any non-zero amount ranging from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation or be any numerical value or subrange within any of these ranges.

In some embodiments where a cell or cell population is present in the pharmaceutical formulation (e.g., as a primary and/or or secondary active agent), the effective amount of cells can be any amount ranging from about 1 or 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL or any numerical value or subrange within any of these ranges.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be about 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L or any numerical value or subrange within these ranges. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more or any numerical value or subrange within these ranges.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent, when optionally present, is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total active agents present in the pharmaceutical formulation or any numerical value or subrange within these ranges. In additional embodiments, the effective amount of the secondary active agent is any non-zero amount ranging from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation or any numerical value or subrange within these ranges.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, 6th Edition, Ansel et al., (Media, PA: Williams and Wilkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof. Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g., metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, mannitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein are part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Methods of Diagnosing B. Burgdorferi Infection

Also described herein are methods of diagnosing, prognosing, and/or monitoring B. burgdorferi infection, symptom thereof, and/or a disease, disorder or condition resulting therefrom in a subject in need thereof. As discussed elsewhere herein when B. burgdorferi infects a host, membrane vesicles are secreted into the host that can contain, inter alia, PAPs, including NapA. Without being bound by theory, those vesicles and/or components thereof can cause or contribute to Lyme's disease pathologies and resulting conditions and/or syndromes. As is discussed elsewhere herein the PAP(s), e.g., NapA, can stimulate a subject's cells, particularly peripheral blood mononuclear cells. Generally, some embodiments of the methods of diagnosing, prognosing, and/or monitoring B. burgdorferi infection, symptom thereof, and/or a disease, disorder or condition resulting therefrom in a subject in need thereof include detecting NapA in a sample from subject in need thereof, and/or detecting IL-17 expression from a subject's peripheral blood mononuclear cells in response to B. burgdorferi infection or contact and/or interaction with a B. burgdorferi component or molecule or composition secreted therefrom and/or molecules contained in or associated therewith, and/or detecting a B. berdoferi reactome expression signature in a subject's peripheral blood mononuclear cells. In some embodiments, the methods described herein are used to diagnose, prognose, and/or monitor Lyme's disease, which is caused by B. burgdorferi infection. As used herein “reactome” is a term of art that refers to the collection of genes, RNA, proteins, epigenetic changes, and/or the like whose expression, production, secretion, and/or the like is changed in response to a reaction and/or the molecular and/or biochemical reactions that take place in response to a stimulus. In the context of the present application, the stimulus for the reactome is infection by B. burgdorferi and or one or more component(s) released therefrom, such as a cell membrane vesicle (e.g., an outer cell membrane vesicle) or component contained in and/or associated with a cell membrane vesicle, such as a PAP (e.g., NapA).

Collectively the molecules detected indicative of B. burgdorferi infection, symptom thereof, and/or a disease, disorder or condition resulting therefrom in a subject in need thereof are biomarkers. Thus, in some embodiments the description herein provides biomarkers (e.g., phenotype specific, cell type specific, reactome specific, etc.) for the identification, diagnosis, prognosis, monitoring, and/or manipulation of cell properties, for use in a variety of diagnostic and/or therapeutic indications and treatment monitoring. Biomarkers in the context of the present invention encompass, without limitation, nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition). The term “monitoring” generally refers to the follow-up of a disease or a condition in a subject for any changes which may occur over time.

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

As used herein, the term “signature” may encompass any gene or genes, protein or proteins, or epigenetic element(s) whose expression profile or whose occurrence is associated with a specific cell type, subtype, or cell state of a specific cell type or subtype within a population of cells. For ease of discussion, when discussing gene expression, any of gene or genes, protein or proteins, or epigenetic element(s) may be substituted. As used herein, the terms “signature”, “expression profile”, or “expression program” may be used interchangeably. It is to be understood that also when referring to proteins (e.g. differentially expressed proteins), such may fall within the definition of “gene” signature. Levels of expression or activity or prevalence may be compared between different cells in order to characterize or identify for instance signatures specific for cell (sub)populations. Increased or decreased expression or activity or prevalence of signature genes may be compared between different cells in order to characterize or identify for instance specific cell (sub)populations. The detection of a signature in single cells may be used to identify and quantitate for instance specific cell (sub)populations. A signature may include a gene or genes, protein or proteins, or epigenetic element(s) whose expression or occurrence is specific to a cell (sub)population, such that expression or occurrence is exclusive to the cell (sub)population. A gene signature as used herein, may thus refer to any set of up- and down-regulated genes that are representative of a cell type or subtype. A gene signature as used herein, may also refer to any set of up- and down-regulated genes between different cells or cell (sub)populations derived from a gene-expression profile. A signature can be composed of any number of genes, proteins epigenetic elements, and/or combinations thereof. For example, a gene signature may include a list of genes differentially expressed in a distinction of interest. The signature can be composed completely of or contain 1, 2, 3, 4, 5, 6, 7, 8, 9, 10, 11, 12, 13, 14, 15, 16, 17, 18, 19, or 20 or more genes, proteins and/or epigenetic elements. In aspects, the signature can be composed completely of or contain 1-20 or more, 2-20 or more, 3-20 or more, 4-20 or more, 5-20 or more, 6-20 or more, 7-20 or more, 8-20 or more, 9-20 or more, 10-20 or more, 11-20 or more, 12-20 or more, 13-20 or more, 14-20 or more, 15-20 or more, 16-20 or more, 17-20 or more, 18-20 or more, 19-20 or more, or 20 or more genes, proteins and/or epigenetic elements.

As used herein “increased expression” or “overexpression” are both used to refer to an increased expression of a gene, such as a gene relating to an antigen processing and/or presentation pathway, or gene product thereof in a sample as compared to the expression of said gene or gene product in a suitable control. The term “increased expression” preferably refers to 10%, 20%, 30%, 40%, 50%, 60%, 70%, 80%, 90%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, 1000%, 1010%, 1020%, 1030%, 1040%, 1050%, 1060%, 1070%, 1080%, 1090%, 1100%, 1110%, 1120%, 1130%, 1140%, 1150%, 1160%, 1170%, 1180%, 1190%, 1200%, 1210%, 1220%, 1230%, 1240%, 1250%, 1260%, 1270%, 1280%, 1290%, 1300%, 1310%, 1320%, 1330%, 1340%, 1350%, 1360%, 1370%, 1380%, 1390%, 1400%, 1410%, 1420%, 1430%, 1440%, 1450%, 1460%, 1470%, 1480%, 1490%, or/to 1500% or more increased expression relative to a suitable control.

As used herein “reduced expression”, “decreased expression”, or “underexpression” refers to a reduced or decreased expression of a gene, such as a gene relating to an antigen processing pathway, or a gene product thereof in sample as compared to the expression of said gene or gene product in a suitable control. As used throughout this specification, “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed. In one embodiment, said control is a sample from a healthy individual or otherwise normal individual. By way of a non-limiting example, if said sample is a sample of a lung tumor and comprises lung tissue, said control is lung tissue of a healthy individual. The term “reduced expression” preferably refers to at least a 25% reduction, e.g., at least a 30%, 40%, 50%, 60%, 70%, 75%, 80%, 85%, 90%, 95%, 98% or 99% reduction, relative to such control.

A “suitable control” is a control that will be instantly appreciated by one of ordinary skill in the art as one that is included such that it can be determined if the variable being evaluated an effect, such as a desired effect or hypothesized effect. One of ordinary skill in the art will also instantly appreciate based on inter alia, the context, the variable(s), the desired or hypothesized effect, what is a suitable or an appropriate control needed.

An altered biomarker in response to B. burgdorferi infection in the subject compared to a suitable control or threshold value status indicates that the subject has an impaired immune status or has a disease, e.g., B. burgdorferi infection, Lyme's disease, symptom thereof, and/or a disease, disorder or condition resulting therefrom comprising or would benefit from a therapy targeting B. burgdorferi or component thereof or secreted therefrom, e.g., NapA or other PAP.

Hence, the methods may rely on comparing the quantity of biomarkers measured in samples from patients with reference values, wherein said reference values represent known predictions, diagnoses and/or prognoses of diseases or conditions as taught herein. For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or ±1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or <90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

Described in certain example embodiments herein are methods of diagnosing, prognosing, monitoring, and/or treating a Borrelia burgdorferi (B. burgdorferi) infection, a symptom thereof, or a disease, disorder or condition resulting therefrom in a subject in need thereof, the method comprising detecting a B. burgdorferi peptidoglycan-associated protein (PAP) in a sample obtained from the subject in need thereof. In certain example embodiments, the B. burgdorferi PAP is neutrophil attracting protein A (NapA). In some embodiments, the method further includes quantifying (via relative or absolute methods) the amount of the PAP, optionally NapA, in the sample.

In certain example embodiments, the sample comprises B. burgdorferi outer membrane vesicles. In certain example embodiments, the PAP is present in the B. burgdorferi outer membrane vesicles. In some embodiments, the method can include isolating outer membrane vesicles from a sample obtained from a subject and detecting, one or more PAPs, optionally NapA in the isolated outer membrane vesicles.

In certain example embodiments, the method further comprises detecting an amount of IL-17 (gene or protein expression) in the sample, wherein an increase in IL-17 gene expression and/or gene product as compared to a suitable control indicates B. burgdorferi infection. In some embodiments, the IL-17 gene and/or gene product expression is detected in peripheral blood mononuclear cells obtained from the subject. In some embodiments the method includes isolating or separating peripheral blood mononuclear cells from the sample obtained from the subject. In some embodiments, IL-17 gene and/or gene product expression is detected in the separated peripheral blood mononuclear cells. Methods of separating cells based on type are generally known in the art. In certain example embodiments, the PAP is neutrophil attracting protein A (NapA). In some embodiments, the IL-17 gene and/or gene product expression in the sample is increased by 1-5000 fold or more, such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, 5000 fold or more as compared to a suitable control.

In certain example embodiments, detecting comprises exposing the sample or component(s) thereof to peripheral blood mononuclear cells in culture and measuring an amount IL-17 in the culture supernatant, whereby an increase in the amount of IL-17 as compared to a suitable control indicates the presence of a PAP, optionally NapA. In some embodiments, the IL-17 in the supernatant is increased by 1-5000 fold or more, such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, 5000 fold or more as compared to a suitable control.

In certain example embodiments, the sample is a bodily fluid, optionally whole blood or fraction thereof (e.g., plasma etc.) or synovial fluid.

In certain example embodiments, detecting comprises a nucleic acid detection method, mass-spectrometry, protein sequencing, an immunodetection method or technique, or any combination thereof.

In some embodiments the method further includes detecting a B. burgdorferi Peptidoglycan reactome signature in peripheral blood mononuclear cells in a sample obtained from a subject. Exemplary embodiments of such detection are described in further detail below.

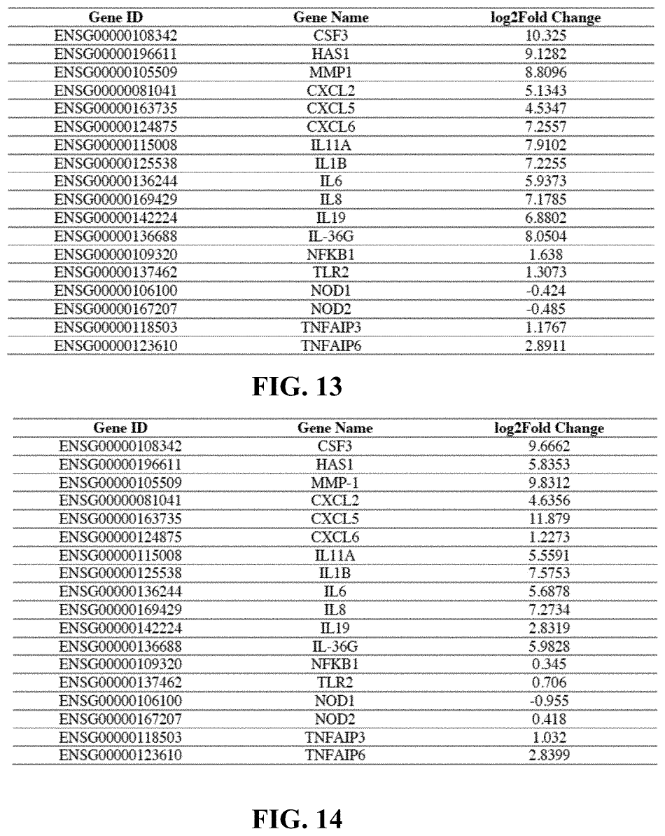

Also provided herein are methods of diagnosing, prognosing, and/or treating B. burgdorferi infection, a symptom thereof, or a disease, disorder or condition resulting therefrom in the subject in need thereof comprising detecting a B. burgdorferi Peptidoglycan reactome signature in peripheral blood mononuclear cells in a sample obtained from a subject. In some embodiments, the reactome signature includes one or more genes, pathways, programs, and/or gene products set forth in FIGS. 13-16, 21, 26-30, 31A-31C, 32A-32B, 33A-33B, 34A-34B, 36A-36B, 37A-37B, 38A-38B, and/or 39 and as further described in Example 2 herein.

In some embodiments, the expression and/or production one or more of the reactome genes and/or gene products are increased, the expression and/or production one or more of the reactome genes and/or gene products are decreased, or both. In some embodiments, the increase in expression of one or more reactome gene or gene products is 1-5,000 fold, such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, 5000 fold or more as compared to a suitable control. In some embodiments, the decrease in expression of one or more reactome gene or gene products is 1-5,000 fold, such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, 5000 fold or more as compared to a suitable control. In some embodiments the decrease in expression of one or more reactome gene or gene products is such that it is below detectable limits.

In certain example embodiments, the method further comprises treating the Borrelia burgdorferi (B. burgdorferi) infection, a symptom thereof, or a disease, disorder or condition resulting therefrom in the subject in need thereof, wherein treating comprises reducing or eliminating a B. burgdorferi peptidoglycan-associated protein (PAP), a function thereof, activity thereof, or any combination thereof in the subject in need thereof. In certain example embodiments, the PAP is neutrophil attracting protein A (NapA).

In some embodiments, treating includes administering a PAP, optionally NapA, inhibitor to the subject in need thereof. In certain example embodiments, treating includes administering to the subject in need thereof comprises administering an antibody or fragment thereof capable of specifically binding the PAP, optionally NapA protein, or an enzyme capable of targeting, degrading, modifying, and/or otherwise inhibiting the PAP, optionally the NapA protein

In certain example embodiments, the B. burgdorferi peptidoglycan-associated protein (PAP), a function thereof, activity thereof, or any combination thereof is reduced 1-5,000 fold, such as 1, 2, 3, 4, 5, 6, 7, 8, 9, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1010, 1020, 1030, 1040, 1050, 1060, 1070, 1080, 1090, 1100, 1110, 1120, 1130, 1140, 1150, 1160, 1170, 1180, 1190, 1200, 1210, 1220, 1230, 1240, 1250, 1260, 1270, 1280, 1290, 1300, 1310, 1320, 1330, 1340, 1350, 1360, 1370, 1380, 1390, 1400, 1410, 1420, 1430, 1440, 1450, 1460, 1470, 1480, 1490, 1500, 1510, 1520, 1530, 1540, 1550, 1560, 1570, 1580, 1590, 1600, 1610, 1620, 1630, 1640, 1650, 1660, 1670, 1680, 1690, 1700, 1710, 1720, 1730, 1740, 1750, 1760, 1770, 1780, 1790, 1800, 1810, 1820, 1830, 1840, 1850, 1860, 1870, 1880, 1890, 1900, 1910, 1920, 1930, 1940, 1950, 1960, 1970, 1980, 1990, 2000, 2010, 2020, 2030, 2040, 2050, 2060, 2070, 2080, 2090, 2100, 2110, 2120, 2130, 2140, 2150, 2160, 2170, 2180, 2190, 2200, 2210, 2220, 2230, 2240, 2250, 2260, 2270, 2280, 2290, 2300, 2310, 2320, 2330, 2340, 2350, 2360, 2370, 2380, 2390, 2400, 2410, 2420, 2430, 2440, 2450, 2460, 2470, 2480, 2490, 2500, 2510, 2520, 2530, 2540, 2550, 2560, 2570, 2580, 2590, 2600, 2610, 2620, 2630, 2640, 2650, 2660, 2670, 2680, 2690, 2700, 2710, 2720, 2730, 2740, 2750, 2760, 2770, 2780, 2790, 2800, 2810, 2820, 2830, 2840, 2850, 2860, 2870, 2880, 2890, 2900, 2910, 2920, 2930, 2940, 2950, 2960, 2970, 2980, 2990, 3000, 3010, 3020, 3030, 3040, 3050, 3060, 3070, 3080, 3090, 3100, 3110, 3120, 3130, 3140, 3150, 3160, 3170, 3180, 3190, 3200, 3210, 3220, 3230, 3240, 3250, 3260, 3270, 3280, 3290, 3300, 3310, 3320, 3330, 3340, 3350, 3360, 3370, 3380, 3390, 3400, 3410, 3420, 3430, 3440, 3450, 3460, 3470, 3480, 3490, 3500, 3510, 3520, 3530, 3540, 3550, 3560, 3570, 3580, 3590, 3600, 3610, 3620, 3630, 3640, 3650, 3660, 3670, 3680, 3690, 3700, 3710, 3720, 3730, 3740, 3750, 3760, 3770, 3780, 3790, 3800, 3810, 3820, 3830, 3840, 3850, 3860, 3870, 3880, 3890, 3900, 3910, 3920, 3930, 3940, 3950, 3960, 3970, 3980, 3990, 4000, 4010, 4020, 4030, 4040, 4050, 4060, 4070, 4080, 4090, 4100, 4110, 4120, 4130, 4140, 4150, 4160, 4170, 4180, 4190, 4200, 4210, 4220, 4230, 4240, 4250, 4260, 4270, 4280, 4290, 4300, 4310, 4320, 4330, 4340, 4350, 4360, 4370, 4380, 4390, 4400, 4410, 4420, 4430, 4440, 4450, 4460, 4470, 4480, 4490, 4500, 4510, 4520, 4530, 4540, 4550, 4560, 4570, 4580, 4590, 4600, 4610, 4620, 4630, 4640, 4650, 4660, 4670, 4680, 4690, 4700, 4710, 4720, 4730, 4740, 4750, 4760, 4770, 4780, 4790, 4800, 4810, 4820, 4830, 4840, 4850, 4860, 4870, 4880, 4890, 4900, 4910, 4920, 4930, 4940, 4950, 4960, 4970, 4980, 4990, 5000 fold or more as compared to a suitable control. Other embodiments for treating B. burgdorferi infection, a symptom thereof, or a disease, disorder or condition resulting therefrom in the subject in need thereof that can be included in the methods herein are described in greater detail elsewhere herein.

### Exemplary Detection Methods and Techniques

Methods of detecting the biomarkers described herein are generally known in the art and can be adapted for use with the present disclosure. For example, in some embodiments, the biomarker(s) can be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

The present invention also may comprise a kit with detection reagent(s) and/or compositions that binds or facilitate binding to one or more biomarkers or can be used to detect one or more biomarkers.

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-associated laser desorption. Common ion sources are, for example, electrospray, including nanospray and microspray or matrix-associated laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-associated laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

In some embodiments, detection includes or is an immunoassay or immunodetection. Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multi-well assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### CRISPR-Cas System Based Nucleic Acid Detection Methods

In some embodiments, a CRISPR-Cas system based method, technique, and/or device can be used to detect a biomarker, such as a nucleic acid biomarker. Exemplary methods and techniques are generally known in the art, including the SHERLOCK and DETECTR methods, which employ collateral cleavage activity of a Cas13 and a Cas12 protein, respectively to cleave a nucleic acid based detection construct in response to binding of the Cas protein and guide RNA complex to a target nucleic acid. For Cas 13 based detection see e.g., in PCT/US18/054472 filed Oct. 22, 2018 at [0183]-[0327], incorporated herein by reference; WO 2017/219027, WO2018/107129, US20180298445, US 2018-0274017, US 2018-0305773, WO 2018/170340, U.S. application Ser. No. 15/922,837, filed Mar. 15, 2018 entitled “Devices for CRISPR Effector System Based Diagnostics”, PCT/US18/50091, filed Sep. 7, 2018 “Multi-Effector CRISPR Based Diagnostic Systems”, PCT/US18/66940 filed Dec. 20, 2018 entitled “CRISPR Effector System Based Multiplex Diagnostics”, PCT/US18/054472 filed Oct. 4, 2018 entitled “CRISPR Effector System Based Diagnostic”, U.S. Provisional 62/740,728 filed Oct. 3, 2018 entitled “CRISPR Effector System Based Diagnostics for Hemorrhagic Fever Detection”, U.S. Provisional 62/690,278 filed Jun. 26, 2018 and U.S. Provisional 62/767,059 filed Nov. 14, 2018 both entitled “CRISPR Double Nickase Based Amplification, Compositions, Systems and Methods”, U.S. Provisional 62/690,160 filed Jun. 26, 2018 and 62/767,077 filed Nov. 14, 2018, both entitled “CRISPR/CAS and Transposase Based Amplification Compositions, Systems, And Methods”, U.S. Provisional 62/690,257 filed Jun. 26, 2018 and 62/767,052 filed Nov. 14, 2018 both entitled “CRISPR Effector System Based Amplification Methods, Systems, And Diagnostics”, U.S. Provisional 62/767,076 filed Nov. 14, 2018 entitled “Multiplexing Highly Evolving Viral Variants With SHERLOCK” and 62/767,070 filed Nov. 14, 2018 entitled “Droplet SHERLOCK.” Reference is further made to WO2017/127807, WO2017/184786, WO 2017/184768, WO 2017/189308, WO 2018/035388, WO 2018/170333, WO 2018/191388, WO 2018/213708, WO 2019/005866, PCT/US18/67328 filed Dec. 21, 2018 entitled “Novel CRISPR Enzymes and Systems”, PCT/US18/67225 filed Dec. 21, 2018 entitled “Novel CRISPR Enzymes and Systems” and PCT/US18/67307 filed Dec. 21, 2018 entitled “Novel CRISPR Enzymes and Systems”, U.S. 62/712,809 filed Jul. 31, 2018 entitled “Novel CRISPR Enzymes and Systems”, U.S. 62/744,080 filed Oct. 10, 2018 entitled “Novel Cas12b Enzymes and Systems” and U.S. 62/751,196 filed Oct. 26 2018 entitled “Novel Cas12b Enzymes and Systems”, U.S. 715,640 filed August 7, 2-18 entitled “Novel CRISPR Enzymes and Systems”, WO 2016/205711, U.S. Pat. No. 9,790,490, WO 2016/205749, WO 2016/205764, WO 2017/070605, WO 2017/106657, and WO 2016/149661, WO2018/035387, WO2018/194963, Cox DBT, et al., RNA editing with CRISPR-Cas13, Science. 2017 Nov. 24; 358(6366):1019-1027; Gootenberg J S, et al., Multiplexed and portable nucleic acid detection platform with Cas13, Cas12a, and Csm6., Science. 2018 Apr. 27; 360(6387):439-444; Gootenberg J S, et al., Nucleic acid detection with CRISPR-Cas13a/C2c2., Science. 2017 Apr. 28; 356(6336):438-442; Abudayyeh 00, et al., RNA targeting with CRISPR-Cas13, Nature. 2017 Oct. 12; 550(7675):280-284; Smargon A A, et al., Cas13b Is a Type VI-B CRISPR-Associated RNA-Guided RNase Differentially Regulated by Accessory Proteins Csx27 and Csx28. Mol Cell. 2017 Feb. 16; 65(4):618-630.e7; Abudayyeh 00, et al., C2c2 is a single-component programmable RNA-guided RNA-targeting CRISPR effector, Science. 2016 Aug. 5; 353(6299):aaf5573; Yang L, et al., Engineering and optimizing deaminase fusions for genome editing. Nat Commun. 2016 Nov. 2; 7:13330, Myrvhold et al., Field deployable viral diagnostics using CRISPR-Cas13, Science 2018 360, 444-448, Shmakov et al. “Diversity and evolution of class 2 CRISPR-Cas systems,” Nat Rev Microbiol. 2017 15(3):169-182, Zhang et al., “Two HPEN domains dictate CRISPR RNA maturation and target cleavage in Cas13d.” Nat. Comm. 10:2544 (2019), Patchsung et al., 2020. Nat. Biomed. Eng. 4:1140-1149; Aquino-Jarquin, G. Drug Discov. Today. 2021. 26(8):2025-2035; Fozouni et al., 2020. Amplification-free detection of SARS-CoV-2 with CRISPR-Cas13a and mobile phone microscopy. Cell. 184:323-333; Lotfi and Rezaei. 2020. CRISPR/Cas13: A potential therapeutic option of COVID-19 Biomedicine & Pharmacotherapy. 131:110738; Khan et al. 2020. CRISPR-Cas13 enzymology rapidly detects SARS-CoV-2 fragments in a clinical setting. medRxiv; doi: https://doi.org/10.1101/2020.12.17.20228593; Schermer et al., Rapid SARS-CoV-2 testing in primary material based on a novel multiplex RT-LAMP assay. PLoS One. https://doi.org/10.1371/journal.pone.0238612; Joung et al., “Detection of SARS-CoV-2 with SHERLOCK One-Pot TestingN Engl J Med 2020; 383:1492-1494” DOI: 10.1056/NEJMc2026172; Joung et al., “Point-of-care testing for COVID-19 using SHERLOCK diagnostics” medRxiv. Preprint. 2020 May 8. doi: 10.1101/2020.05.04.20091231; WO 2017/218573; US 20200010878; US 20200010879; US 20190177775; US 20180208977; US 20180208976; US 20190177775; U.S. Provisional Application Ser. No. 62/351,172; the disclosure of each can be adapted for use with the present invention in view of the description provided herein and each of which is incorporated herein by reference in its entirety. For exemplary Cas 12 based detection methods and devices see e.g., Broughton et al. 2020. CRISPR-Cas12-based detection of SARS-CoV-2. Nat. Biotech. 38:870-874, https://doi.org/10.1038/s41587-020-0513-4; Leung et al. 2021. CRISPR-Cas12-based nucleic acids detection systems. Methods.; 51046-2023(21)00063-3.doi: 10.1016/j.ymeth.2021.02.018; Mahas et al., Viruses. 2021. 13:466, https://doi.org/10.3390/v13030466; Ali et al., 2020. iSCAN: An RT-LAMP-coupled CRISPR-Cas12 module for rapid, sensitive detection of SARS-CoV-2Vir. Res. 288:198129. https://doi.org/10.1016/j.virusres.2020.198129; Ramachandran et al., 2020. Electric field-driven microfluidics for rapid CRISPR-based diagnostics and its application to detection of SARS-CoV-2. PNAS Nov. 24, 2020 117 (47) 29518-29525; Mukama et al., An ultrasensitive and specific point-of care CRISPR-Cas12 based lateral flow biosensor for the rapid detection of nucleic acids. Biosens Bioelectron. 2020 Jul. 1; 159:112143. doi: 10.1016/j.bios.2020.112143; Chen et al., 2018. CRISPR-Cas12a target binding unleashes indiscriminate single-stranded DNase activity. Science. April 27; 360(6387):436-439. doi: 10.1126/science.aar6245; Kellner et al., 2019. Nat Protoc. 2019 October; 14(10):2986-3012. doi: 10.1038/s41596-019-0210-2; Broughton et al., 2020. Rapid Detection of 2019 Novel Coronavirus SARS-CoV-2 Using a CRISPR-based DETECTR Lateral Flow Assay. 2020. medRxiv. March 27; 2020.03.06.20032334. doi: 10.1101/2020.03.06.20032334; Wu et al. 2021. CRISPR-Cas12-Based Rapid Authentication of Halal Food. J Agric Food Chem. 2021 Aug. 26. doi: 10.1021/acs.jafc.1c03078; Long et al. 2021. CRISPR/Cas12-Based Ultra-Sensitive and Specific Point-of-Care Detection of HBV. Int J Mol Sci. 2021 May 3; 22(9):4842. doi: 10.3390/ijms22094842; Curti et al., Viruses. 2021 Mar. 5; 13(3):420. doi: 10.3390/v13030420; Li et al., Cell Discovery (2018)4:20. DOI 10.1038/s41421-018-0028-z; Lucia et al. 2020. An ultrasensitive, rapid, and portable coronavirus SARS-Cov-2 sequence detection method based on CRISPR-Cas12. bioRxiv preprint doi: https/doi.org/10.1101/2020.02.29.971127; MammothBiosciences. 2020. Broughton et al., available at https://mammoth.bio/wp-content/uploads/2020/04/200423-A-protocol-for-rapid-detection-of-SARS-CoV-2-using-CRISPR-diagnostics_3.pdf; East-Seletsky et al., Nat. 538:270, doi:10.1038/nature19802; International Pat. Pub. WO2019/233358; WO2019/011022; U.S. Pat. Nos. 10,337,051; 10,449,4664, 10,253,365; US 2020/0299768; US 2020/0399697; US 2019/0241954; the disclosure of each can be adapted for use with the present invention in view of the description provided herein and each of which is incorporated herein by reference in its entirety.

Digital Nucleic Acid Detection and/or Quantification Methods

As used herein, digital nucleic acid detection” refers to nucleic acid detection approaches that include amplifying nucleic acid amplification, sequencing, and the like with a digital readout. Exemplary techniques include, but are not limited to, SlipChip mediated methods (see e.g., Shen, F. Methods Mol Biol. 2017; 1547:123-132.) doi: 10.1007/978-1-4939-6734-6_10), Nanostring (see e.g., Kulkarni, M. M. Curr Protoc Mol Biol. 2011 April; Chapter 25:Unit25B.10. doi: 10.1002/0471142727.mb25b10s94), and others such as those in described in Xiong and Ying. Chin J Anal Chem, 2016, 44(4), 512-521, dPCR techniques, dLAMP (see e.g., Lin et al., ACS Sens. 2019, 4, 242-249). Other digital nucleic acid detection methods that can be adapted for use with the embodiments described herein will be appreciated by those of ordinary skill in the art

### Probe-Based Polynucleotide Detection

In some embodiments, detection includes or is a probe-based nucleic acid detection assay. In some embodiments, the probe-based assay can include using one or more labeled probes. Generally, probe-based assays can selectively qualitatively or quantitatively detect a target polynucleotide, such as a replication-specific feature described herein based on the ability of the probe to specifically bind or otherwise detectably-interact with a target molecule. Various probe-based assays are known in the art and are described below and elsewhere herein. For example, PCR-based assays are example probe-based assays in which primers, for example, can act as sequence specific probes that result in the sequence specific amplification of a target molecule. In some probe-based methods include labeled oligo probes that specifically bind a target molecule and include a detectable label that provides the ability to detect the target molecule. In some embodiments, complexes such as a CRISPR-Cas based system that includes a guideRNA “probe” can be used to detect a target polynucleotide. In some embodiments the probe-based CRISPR-effector system can include a dCas molecule that facilitates guideRNA binding to a target molecule but does not cleave the target molecule. In some embodiments the dCas can be labeled with a detectable label. In some embodiments, the Cas included in the CRISPR-Cas based system can have collateral activity that can act on a reporter molecule when bound to a target molecule. Such systems are described in greater detail elsewhere herein. Exemplary CRISPR-Cas based systems are described elsewhere herein.

As used herein, the term “specific binding” can refer to non-covalent physical association of a first and a second moiety wherein the association between the first and second moieties is at least 2 times as strong, at least 5 times as strong as, at least 10 times as strong as, at least 50 times as strong as, at least 100 times as strong as, or stronger than the association of either moiety with most or all other moieties present in the environment in which binding occurs. Binding of two or more entities may be considered specific if the equilibrium dissociation constant, Kd, is 10⁻³ M or less, 10⁻⁴ M or less, 10⁻⁵ M or less, 10⁻⁶ M or less, 10⁻⁷ M or less, 10⁻⁸M or less, 10⁻⁹ M or less, 10⁻¹⁰ M or less, 10⁻¹¹ M or less, or 10⁻¹² M or less under the conditions employed, e.g., under physiological conditions such as those inside a cell or consistent with cell survival. In some embodiments, specific binding can be accomplished by a plurality of weaker interactions (e.g., a plurality of individual interactions, wherein each individual interaction is characterized by a Kd of greater than 10⁻³ M). In some embodiments, specific binding, which can be referred to as “molecular recognition,” is a saturable binding interaction between two entities that is dependent on complementary orientation of functional groups on each entity. Examples of specific binding interactions include primer-polynucleotide interaction, aptamer-aptamer target interactions, antibody-antigen interactions, avidin-biotin interactions, ligand-receptor interactions, metal-chelate interactions, hybridization between complementary nucleic acids, etc.

As used herein, “probe” refers to agent capable of specifically binding (such as by hybridizing) or otherwise detectably interacting with a target molecule (such as a nucleic acid). In some embodiments, the target molecule is a replication-specific feature. A detectable label or reporter molecule can be attached to a probe. Typical labels include radioactive isotopes, enzyme substrates, co-factors, ligands, chemiluminescent or fluorescent agents, haptens, and enzymes. As used herein, “label” refers to an agent capable of detection, for example by spectrophotometry, flow cytometry, or microscopy. For example, a label can be attached to a nucleotide, thereby permitting detection of the nucleotide, such as detection of the nucleic acid molecule of which the nucleotide is a part. Examples of labels include, but are not limited to, radioactive isotopes, enzyme substrates, co-factors, ligands, chemiluminescent agents, fluorophores, haptens, enzymes, and combinations thereof. Methods for labeling and guidance in the choice of labels appropriate for various purposes are discussed for example in Sambrook et al. (Molecular Cloning: A Laboratory Manual, Cold Spring Harbor, N.Y., 1989) and Ausubel et al. (In Current Protocols in Molecular Biology, John Wiley & Sons, New York, 1998).

Methods for labeling and guidance in the choice of labels appropriate for various purposes are discussed, for example, in Sambrook et al., Molecular Cloning: A Laboratory Manual, Cold Spring Harbor Laboratory Press (1989) and Ausubel et al., Current Protocols in Molecular Biology, Greene Publishing Associates and Wiley-Intersciences (1987). In a particular example, a probe includes at least one fluorophore, such as an acceptor fluorophore or donor fluorophore. For example, a fluorophore can be attached at the 5′- or 3′-end of the probe. In specific examples, the fluorophore is attached to the base at the 5′-end of the probe, the base at its 3′-end, the phosphate group at its 5′-end or a modified base, such as a T internal to the probe.

Probes are generally about 15 nucleotides in length to about 160 nucleotides in length, such as 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160 contiguous nucleotides complementary to the target nucleic acid molecule, such as 50-140 nucleotides, 75-150 nucleotides, 60-70 nucleotides, 30-130 nucleotides, 20-60 nucleotides, 20-50 nucleotides, 20-40 nucleotides, 20-30 nucleotides, or 40 to 60 nucleotides.

As used herein, “fluorophore” refers to A chemical compound, which when excited by exposure to a particular stimulus such as a defined wavelength of light, emits light (fluoresces), for example at a different wavelength (such as a longer wavelength of light). Fluorophores are part of the larger class of luminescent compounds. Luminescent compounds include chemiluminescent molecules, which do not require a particular wavelength of light to luminesce, but rather use a chemical source of energy. Therefore, the use of chemiluminescent molecules (such as aequorin) eliminates the need for an external source of electromagnetic radiation, such as a laser. Examples of particular fluorophores that can be used in the probes disclosed herein are provided in U.S. Pat. No. 5,866,366 to Nazarenko et al., such as 4-acetamido-4′-isothiocyanatostilbene-2,2′disulfonic acid, acridine and derivatives such as acridine and acridine isothiocyanate, 5-(2′-aminoethyl)aminonaphthalene-1-sulfonic acid (EDANS), 4-amino-N-[3-vinylsulfonyl)phenyl]naphthalimide-3,5 disulfonate (Lucifer Yellow VS), N-(4-anilino-1-naphthyl)maleimide, anthranilamide, Brilliant Yellow, coumarin and derivatives such as coumarin, 7-amino-4-methylcoumarin (AMC, Coumarin 120), 7-amino-4-trifluoromethylcouluarin (Coumaran 151); cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-di sulfonic acid; 4,4′-diisothiocyanatostilbene-2,2′-disulfonic acid; 5-[dimethylamino]naphthalene-1-sulfonyl chloride (DNS, dansyl chloride); 4-dimethylaminophenylazophenyl-4′-isothiocyanate (DABITC); eosin and derivatives such as eosin and eosin isothiocyanate; erythrosin and derivatives such as erythrosin B and erythrosin isothiocyanate; ethidium; fluorescein and derivatives such as 5-carboxyfluorescein (FAM), 5-(4,6-dichlorotriazin-2-yl)aminofluorescein (DTAF), 2′7′-dimethoxy-4′5′-dichloro-6-carboxyfluorescein (JOE), fluorescein, fluorescein isothiocyanate (FITC), and QFITC (XRITC); fluorescamine; IR144; IR1446; Malachite Green isothiocyanate; 4-methylumbelliferone; ortho cresolphthalein; nitrotyrosine; pararosaniline; Phenol Red; B-phycoerythrin; o-phthaldialdehyde; pyrene and derivatives such as pyrene, pyrene butyrate and succinimidyl 1-pyrene butyrate; Reactive Red 4 (Cibacron® Brilliant Red 3B-A); rhodamine and derivatives such as 6-carboxy-X-rhodamine (ROX), 6-carboxyrhodamine (R6G), lissamine rhodamine B sulfonyl chloride, rhodamine (Rhod), rhodamine B, rhodamine 123, rhodamine X isothiocyanate, sulforhodamine B, sulforhodamine 101 and sulfonyl chloride derivative of sulforhodamine 101 (Texas Red); N,N,N′,N′-tetramethyl-6-carboxyrhodamine (TAMRA); tetramethyl rhodamine; tetramethyl rhodamine isothiocyanate (TRITC); riboflavin; rosolic acid and terbium chelate derivatives; LightCycler Red 640; Cy5.5; and Cy56-carboxyfluorescein; 5-carboxyfluorescein (5-FAM); boron dipyrromethene difluoride (BODIPY); N,N,N′,N′-tetramethyl-6-carboxyrhodamine (TAMRA); acridine, stilbene, -6-carboxy-fluorescein (HEX), TET (Tetramethyl fluorescein), 6-carboxy-X-rhodamine (ROX), Texas Red, 2′,7′-dimethoxy-4′,5′-dichloro-6-carboxyfluorescein (JOE), Cy3, Cy5, VIC® (Applied Biosystems), LC Red 640, LC Red 705, Yakima yellow amongst others.

Other suitable fluorophores include those known to those skilled in the art, for example those available from Molecular Probes (Eugene, Oreg.). In particular embodiments, a fluorophore is used as a donor fluorophore or as an acceptor fluorophore. “Acceptor fluorophores” are fluorophores which absorb energy from a donor fluorophore, for example in the range of about 400 to 900 nm (such as in the range of about 500 to 800 nm). Acceptor fluorophores generally absorb light at a wavelength which is usually at least 10 nm higher (such as at least 20 nm higher), than the maximum absorbance wavelength of the donor fluorophore, and have a fluorescence emission maximum at a wavelength ranging from about 400 to 900 nm. Acceptor fluorophores have an excitation spectrum which overlaps with the emission of the donor fluorophore, such that energy emitted by the donor can excite the acceptor. Ideally, an acceptor fluorophore is capable of being attached to a nucleic acid molecule.

In a particular example, an acceptor fluorophore is a dark quencher, such as, Dabcyl, QSY7 (Molecular Probes), QSY33 (Molecular Probes), BLACK HOLE QUENCHERS™ (Glen Research), ECLIPSE™ Dark Quencher (Epoch Biosciences), IOWA BLACK™ (Integrated DNA Technologies). A quencher can reduce or quench the emission of a donor fluorophore. In such an example, instead of detecting an increase in emission signal from the acceptor fluorophore when in sufficient proximity to the donor fluorophore (or detecting a decrease in emission signal from the acceptor fluorophore when a significant distance from the donor fluorophore), an increase in the emission signal from the donor fluorophore can be detected when the quencher is a significant distance from the donor fluorophore (or a decrease in emission signal from the donor fluorophore when in sufficient proximity to the quencher acceptor fluorophore). “Donor Fluorophores” are fluorophores or luminescent molecules capable of transferring energy to an acceptor fluorophore, thereby generating a detectable fluorescent signal from the acceptor. Donor fluorophores are generally compounds that absorb in the range of about 300 to 900 nm, for example about 350 to 800 nm. Donor fluorophores have a strong molar absorbance coefficient at the desired excitation wavelength, for example greater than about 10³ M⁻¹ cm⁻¹.

The method comprises, for example, contacting a sample comprising or suspected of comprising the target RNA (such as mRNA, tTNA and rRNA,), such as a sample obtained from a subject, such as a human subject, or an environmental sample, or food source sample, with a set of probes that are antisense, e.g., anneal to a target or set of target RNAs. The set of probes comprises at least one detectable probe that is specific for a target RNA sequence of each species to be tested, wherein the individual probes specific for each species have about 85% or less sequence identity to the probes for the other species, such as less than about 75%, less than about 70%, or even less than about 50% sequence identity to the probes for the other species. The method further comprises detecting hybridization between one or more of the probes and the RNA, thereby distinguishing between two or more species in a sample. In certain example embodiments, the individual probes specific for each species have about 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60% or less sequence identity to the probes for the other species.

Hybridization between one or more of the probes and the target RNAs is detected, thereby distinguishing between two or more species in a sample. In some embodiments, detecting hybridization between the probe indicates the presence of the species in the sample, and can indicate that the subject from which, the sample was obtained is infected and/or contaminated with the organism.

In some embodiments, a target polynucleotide can be detected, for example, example using Nanostring's method of two DNA oligonucleotides that bind to adjacent 50-nucleotide stretches of RNA. When the target transcript is present in a lysate, it links a biotinylated DNA oligo (the “capture probe”) to an adjacent fluorescently labeled DNA oligo (the “reporter probe”). In particular embodiments, two molecular probes are added to a crude sample lysate. A capture probe comprises 50 nucleotides complementary to a given RNA molecule and can be conjugated to biotin. A reporter probe comprises a different 50 nucleotides complementary to a different part of the same RNA molecule, and can be conjugated to a reporter molecule, e.g., a fluorescent tag or quantum dot. Each reporter probe uniquely identifies a given RNA molecule. The capture and reporter probes hybridize to their corresponding RNA molecules within the lysate. Excess reporter is removed by bead purification that hybridizes to a handle on each oligomer, leaving only the hybridized RNA complexes. The RNA complexes can be captured and immobilized on a surface, e.g., a streptavidin-coated surface. An electric field can be applied to align the complexes all in the same direction on the surface before the surface is microscopically imaged.

In some embodiments, the technique employs a commercial RNA recognition technology known as NanoString, which has largely been applied to characterization of messenger RNA (mRNA) transcripts. The reporter probes can be counted to provide a quantitative measure of RNA molecules. A commercially available nCounter® Analysis System (NanoString, Seattle, Wash.) can be used in the procedure. It will be understood by those skilled in the art, that other systems may be used in the process.

In some embodiments of the disclosed methods, determining the identity of a nucleic acid includes detection by nucleic acid hybridization. Nucleic acid hybridization involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids. Under low stringency conditions (e.g., low temperature and/or high salt) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches. One of skill in the art will appreciate that hybridization conditions can be designed to provide different degrees of stringency.

In general, there is a tradeoff between hybridization specificity (stringency) and signal intensity. Thus, in one embodiment, the wash is performed at the highest stringency that produces consistent results and that provides a signal intensity greater than approximately 10% of the background intensity. Thus, the hybridized array may be washed at successively higher stringency solutions and read between each wash. Analysis of the data sets thus produced will reveal a wash stringency above which the hybridization pattern is not appreciably altered and which provides adequate signal for the particular oligonucleotide probes of interest. In some examples, RNA is detected using Northern blotting or in situ hybridization (Parker & Barnes, Methods in Molecular Biology 106:247-283, 1999); RNase protection assays (Hod, Biotechniques 13:852-4, 1992); and PCR-based methods, such as reverse transcription polymerase chain reaction (RT-PCR) (Weis et al., Trends in Genetics 8:263-4, 1992).

In some examples, nucleic acids are identified or confirmed using the microarray technique.

Means of detecting such labels are also well known. Thus, for example, radiolabels may be detected using photographic film or scintillation counters. Fluorescent markers may be detected using a photodetector to detect emitted light. Enzymatic labels are typically detected by providing the enzyme with a substrate and detecting the reaction product produced by the action of the enzyme on the substrate, and colorimetric labels are detected by simply visualizing the colored label.

The label may be added to the target (sample) nucleic acid(s) prior to, or after, the hybridization. So-called “direct labels” are detectable labels that are directly attached to or incorporated into the target (sample) nucleic acid prior to hybridization. In contrast, so-called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected (see Laboratory Techniques in Biochemistry and Molecular Biology, Vol. 24: Hybridization With Nucleic Acid Probes, P. Tij ssen, ed. Elsevier, N.Y., 1993).

In some embodiments, the probe(s) can be attached or included in an array. Arrays are described in greater detail elsewhere herein.

In some embodiments, the probe-based assay is an electrochemical-based assay. Such assays are described in greater detail elsewhere herein.

In some embodiments, the probe-based assay is a thermal based assay, such as that described in U.S. Pat. No. 9,995,680, which can be adapted for use with the various embodiments described herein.

In some embodiments, the probe-based assay can be performed on filter paper, such as in the context of a POC device, as described in e.g., Song and Gyarmati. 2020. New Biotech. 55:77-83, which can be adapted for use with the various embodiments described herein.

### Electrochemistry-Based Polynucleotide Detection

In some embodiments, detecting can include or be an electrochemistry-based polynucleotide detection method. Electrochemical methods of polynucleotide detection are selective and sensitive techniques for detecting and distinguishing polynucleotides with specific sequences and/or features such as methylation and/or phosphorylation. Generally, these methods include coupling a capture probe specific to a polynucleotide sequence of interest to an electrode such that when a target polynucleotide binds to the capture probe a change in the electric potential of the substrate is generated thus forming a detectable signal. There are many variations to the basic principle of the detection method as will be appreciated by those of ordinary skill in the relevant art. Exemplary electrochemistry-based polynucleotide detection methods include, but are not limited to, those described and discussed in any of the following: WO/1993/020230 WO2017/026901, WO/2004/094986, WO/2018/087303, WO/2018/197725; WO/2003/023365; WO/2005/001122; WO/2002/063041; U.S. Pat. Nos. 7,820,030; 8,105,477; 9,234,867; 8,425,745; 9,612,222; 9,624,532; 10,465,244; U.S. Pat. App. Publications 2007/0099211; 2014/0102915; 20190048402, which can be adapted for use with the various embodiments described herein.

### Electric Field Associated Polynucleotide Detection

In some embodiments, an electric field associated polynucleotide detection method can be used to detect one or more polynucleotides. Exemplary electric field-associated polynucleotide detection methods include, but are not limited, to those described in e.g., Nanotechnology 16(10):2061-71 (October 2005), which can be adapted for use with the various embodiments described herein.

### Microscopy Associated Polynucleotide Detection

In some embodiments, the polynucleotide detection method can be or include a microscopy associated polynucleotide detection method. As used herein, “microscopy associated polynucleotide detection” refers to detection methods that utilize a microscope to assist with detection of a target polynucleotide. In some embodiments, the microscope is capable of detecting an optical signal output. Such methods can include reading signals from chips, plates, beads, tissues, and the like. In some embodiments, the microscope can be a transmission or scanning electron microscope. Exemplary methods include, but are not limited to, Nanostring method, U.S. Pat. Nos. 7,381,529, 9,777,313, Moller et al., Nucleic Acids Res. 2000 Oct. 15; 28(20): e91; Wei at al., 2014. ACS. 8(12): 12725-12733.

### In-Situ Polynucleotide Detection

In some embodiments, the polynucleotide detection method is or includes an in-situ polynucleotide detection method. Exemplary in-situ polynucleotide detection methods include, but are not limited to, probe-based or other hybridization methods conducted in situ, sequencing methods (see e.g., Lee et al. Nature Protocols volume 10, pages 442-458 (2015) (FISSEQ), Gyllborg et al. BioRxiv. doi: https://doi.org/10.1101/2020.02.03.931618 (HybISS), Furth et al. BioRxiv. doi: https://doi.org/10.1101/722819 (INSTA-eq), Wang et al., Science. 2018. 361:328. DOI: 10.1126/science.aat5691, Chen at al. 2020. Cell. https://doi.org/10.1016/j.ce11.2020.06.038.

### Array-Based Polynucleotide Detection

In some embodiments, the detection (or sequencing) method includes performing the method in an array. One or more locations on/in the array can contain one or more reagents, probes, effector systems, compositions, molecules, and the like for performing the detection and/or sequencing method described elsewhere herein within one or more locations in the array.

As used herein, “array” encompasses any two- or three-dimensional ordered arrangement of features, where each feature has a unique position in two- or three-dimensional space. Thus, it will be appreciated that each feature in an array can be identified by a unique x,y (two-dimensional arrays) or unique x,y,z coordinate (three-dimensional arrays). Each feature of the array can be any physical, chemical, or biological, composition, property, or aspect that can or has the potential to bind with, react with, contain, fixate, incorporate, or otherwise hold in position a sample or a component thereof. As used herein, “addressable array” refers to an array where the unique position of each feature is predetermined and/or is organized such that each feature and/or its position is otherwise identifiable from the each other feature and/or position thereof. Such predetermined and/or organized addressing of the features in an addressable array can allow for detection, measuring, determination, and/or identification of e.g., a specific target present in a sample, a specific sample characteristic(s) or response(s) present in a sample, a specific condition or set of conditions applied at each feature that elicits or causes a response in a sample, or any combination thereof, thus providing useable information about the sample or one or more component thereof and/or condition(s) applied to a sample.

Features can be arranged within an array such that there is substantially no distance between two or more features, that there is a distance between two or more features, or a combination thereof. In some embodiments, the distance between each feature is the same between each feature of the array. In some embodiments, the distance between each feature of the array can be varied. In some embodiments, the features can be contained in, attached to, integrated with, or otherwise coupled to a substrate or a surface thereof.

In some embodiments, one or more of the features can contain one or more sub features. The sub features can be contained in, attached to, integrated with, or otherwise coupled to the feature and/or substrate or a surface thereof. As used herein, “attached” can refer to covalent or non-covalent interaction between two or more molecules. Non-covalent interactions can include ionic bonds, electrostatic interactions, van der Walls forces, dipole-dipole interactions, dipole-induced-dipole interactions, London dispersion forces, hydrogen bonding, halogen bonding, electromagnetic interactions, π-π interactions, cation-π interactions, anion-π interactions, polar π-interactions, and hydrophobic effects. In some embodiments, the features can be adsorbed, physisorbed, or chemisorbed to a substrate. In some embodiments, the substrate can fix or hold the feature in a specific position within the array. In some embodiments, the features can be formed from voids present in the substrate (e.g., wells or etchings). In some embodiments, the sub features can be adsorbed, physisorbed, or chemisorbed to a substrate. In some embodiments, the substrate can fix or hold the sub feature in a specific position within the feature of the array. In some embodiments, the sub features can be formed from voids present in a feature (e.g., void, engraving or etching). Sub features can be arranged within a feature of the array such that there is substantially no distance between two or more sub features, that there is a distance between two or more sub features, or a combination thereof. In some embodiments, the distance between each sub feature is the same between each sub feature. In some embodiments, the distance between each sub feature of the array can be varied. In some embodiments, the sub features can be contained in, attached to, integrated with, or otherwise coupled to a feature, the substrate and/or a surface thereof.

Further aspects of the array are discussed in greater detail elsewhere herein.

### Array Substrate

The substrate can be solid, vitreous solid, semisolid, liquid, gel, hydrogel, or any permissible combination thereof. As used herein, “hydrogel” refers to a gelatinous colloid, or aggregate of polymeric molecules in a finely dispersed semi-solid state, where the polymeric molecules are in the external or dispersion phase and water (or an aqueous solution) is forms the internal or dispersed phase. Generally, hydrogels are at least 90% by weight of an aqueous solution. The substrate can be any permissible shape or size. In some embodiments the substrate can be or have a regular shape. In some embodiments the substrate can have an irregular shape. The substrate can have any useful form including beads, bottles, planar objects (e.g., slides, plates, etc.), matrices, containers, vessels, dishes, fibers, wafers, plates (e.g. single well plates, multi-well plates, etched, engraved, etc.), chips, membranes, particles, microparticles, sticks, strips, thin films, tapes, fibers, tubes, chambers, droplets, capillaries, or any combination thereof.

A substrate can contain a single array or can contain multiple arrays. In some embodiments, a substrate can contain a single addressable array. In some embodiments, the substrate can contain multiple addressable arrays.

In some embodiments, one or more dimensions of the substrate (e.g., a length, a width, a height, a diameter, and the like) can range from about 1-1,000 pm, nm, μm, cm, or mm. In some embodiments, one or more dimensions of the substrate can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the substrate can range from 1-1,000 pm, nm, μm, cm, or mm. In some embodiments, the largest dimension of the substrate can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the substrate can range from 1-1,000 pm, nm, μm, cm, or mm. In some embodiments, the smallest dimension of the substrate can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, the substrate can have a volume. The volume of the substrate can range from about 1-1,000 pm³, nm³, μm³, cm³, mm³, or L³. In some embodiments, the substrate volume can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, the features are attached or otherwise coupled on one or more surfaces of the substrate. As used herein, “surface,” in the context herein, refers to a boundary of an object, such as the substrate. The surface can be an interior surface (e.g., the interior boundary of a hollow object), or an exterior or outer boundary of a substrate. Generally, the surface of a substrate corresponds to the idealized surface of a three dimensional solid that is topological homeomorphic with the substrate. The surface can be an exterior surface or an interior surface. An exterior surface forms the outermost layer of a substrate or device. An interior surface surrounds an inner cavity of a substrate or device, such as the inner cavity of a tube. As an example, both the outside surface of a tube and the inside surface of a tube are part of the surface of the tube. In some embodiments, one or more surfaces can be modified with one or more features. In some embodiments, one or more surfaces can be functionalized to facilitate attachment or coupling of one or more features to the surface.

In some embodiments, one or more dimensions of the surface (e.g., a length, a width, a height, a diameter, and the like) can range from about 1-1,000 pm, nm, μm, cm, or mm. In some embodiments, one or more dimensions of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the surface can range from 1-1,000 pm, nm, μm, cm, or mm. In some embodiments, the largest dimension of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the surface can range from about 1-1,000 pm, nm, μm, cm, or mm. In some embodiments, the smallest dimension of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments the surface area of the surface can range about 1-1,000 pm², nm², μm², cm², or mm². In some embodiments, the surface area of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm²,nm², cm², or mm².

In some embodiments, the surface can have a volume. The volume of the surface can range from about 1-1,000 pm³, nm³, μm³, cm³, mm³, or C. In some embodiments, the substrate volume can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments the surface and/or substrate can be porous. In some embodiments the pores of the surface and/or substrate can be substantially homogenous. In some embodiments the pores of the surface and/or substrate can be heterogenous. Pores can have any irregular or regular shape. In some embodiments the surface and/or substrate a population of pores can have an average diameter, average largest dimension, and/or average smallest dimension that can range from 1-1,000 pm, nm, μm, cm, or mm. In some embodiments, the average diameter, average largest dimension, and/or average smallest dimension of the a population of pores can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, one or more pores of the substrate and/or surface can have a diameter, a largest dimension, and/or a smallest dimension that can range from about 1-1,000 pm, nm, μm, cm, or mm. In some embodiments one or more pores of the substrate and/or surface can have a diameter, a largest dimension, and/or a smallest dimension that can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments, the population of pores of the substrate and/or surface can have a total pore volume. In some embodiments, the total pore volume of the substrate and/or surface can range from 1-1,000 pm³, nm³, μm³, cm³, mm³, or L³. In some embodiments, the total poor volume can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, all or one or more parts of the substrate and/or surface can be opaque. In some embodiments, all or one or more parts of the substrate and/or surface can be transparent. In some embodiments, all or one or more parts of the substrate and/or surface can be semi-transparent.

The substrate and/or surface can be completely composed of or include any suitable material(s). Suitable materials include, but are not limited to, glass, ceramics, polymers, gels, hydrogels, adhesives, metals, metalloids, metal alloys, non-metals, crystals, fibrous material, and combinations thereof. The substrate and/or surface can be composed of a biocompatible material.

The term “biocompatible”, as used herein, refers to a substance or object that performs its desired function when introduced into an organism without inducing significant inflammatory response, immunogenicity, or cytotoxicity to native cells, tissues, or organs, or to cells, tissues, or organs introduced with the substance or object. For example, a biocompatible product is a product that performs its desired function when introduced into an organism without inducing significant inflammatory response, immunogenicity, or cytotoxicity to native cells, tissues, or organs.

Biocompatibility, as used herein, can be quantified using the following in vivo biocompatibility assay. A material or product is considered biocompatible if it produces, in a test of biocompatibility related to immune system reaction, less than 50%, 45%, 40%, 35%, 30%, 25%, 20%, 15%, 10%, 8%, 6%, 5%, 4%, 3%, 2%, or 1% of the reaction, in the same test of biocompatibility, produced by a material or product the same as the test material or product except for a lack of the surface modification on the test material or product. Examples of useful biocompatibility tests include measuring and assessing cytotoxicity in cell culture, inflammatory response after implantation (such as by fluorescence detection of cathepsin activity), and immune system cells recruited to implant (for example, macrophages and neutrophils).

As used herein, “polymer” refers to molecules made up of monomers repeat units linked together. “Polymers” are understood to include, but are not limited to, homopolymers, copolymers, such as for example, block, graft, random and alternating copolymers, terpolymers, etc. and blends and modifications thereof. “A polymer” can be a three-dimensional network (e.g., the repeat units are linked together left and right, front and back, up and down), a two-dimensional network (e.g., the repeat units are linked together left, right, up, and down in a sheet form), or a one-dimensional network (e.g., the repeat units are linked left and right to form a chain). “Polymers” can be composed, natural monomers or synthetic monomers and combinations thereof. The polymers can be biologic (e.g., the monomers are biologically important (e.g., an amino acid), natural, or synthetic. As used interchangeably herein, “polymer blend” and “polymer mixture” refers to a macroscopically homogenous mixture of two or more different species of polymers. Unlike a copolymer, where the monomeric polymers are covalently linked, the constituents of a “polymer blend” and “polymer mixture” are separable by physical means and does not require covalent bonds to be broken. A “polymer blend” can have 2 or more (e.g., 2, 3, 4, 5, 6, 7, 8, 9, 10 or more) different polymer constituents.

Exemplary synthetic polymers include, without limitation, poly(hydroxy acids) such as poly(lactic acid), poly(glycolic acid), and poly(lactic acid-co-glycolic acid), poly(lactide), poly(glycolide), poly(lactide-co-glycolide), polyanhydrides, polyorthoesters, polyamides, polycarbonates, polyalkylenes such as polyethylene and polypropylene, polyalkylene glycols such as poly(ethylene glycol), polyalkylene oxides such as poly(ethylene oxide), polyalkylene terepthalates such as poly(ethylene terephthalate), polyvinyl alcohols, polyvinyl ethers, polyvinyl esters, polyvinyl halides such as poly(vinyl chloride), polyvinylpyrrolidone, polysiloxanes, poly(vinyl alcohols), poly(vinyl acetate), polystyrene, polyurethanes and co-polymers thereof, derivatized celluloses such as alkyl cellulose, hydroxyalkyl celluloses, cellulose ethers, cellulose esters, nitro celluloses, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, and cellulose sulphate sodium salt (jointly referred to herein as “synthetic celluloses”), polymers of acrylic acid, methacrylic acid or copolymers or derivatives thereof including esters, poly(methyl methacrylate), poly(ethyl methacrylate), poly(butylmethacrylate), poly(isobutyl methacrylate), poly(hexylmethacrylate), poly(isodecyl methacrylate), poly(lauryl methacrylate), poly(phenyl methacrylate), poly(methyl acrylate), poly(isopropyl acrylate), poly(isobutyl acrylate), and poly(octadecyl acrylate) (jointly referred to herein as “polyacrylic acids”), poly(butyric acid), poly(valeric acid), and poly(lactide-co-caprolactone), copolymers and blends thereof. As used herein, “derivatives” include polymers having substitutions, additions of chemical groups, for example, alkyl, alkylene, hydroxylations, oxidations, and other modifications routinely made by those skilled in the art.

As used herein, “glass” refers to any type of glass including, but not limited to silicate glasses (e.g., soda-lime glass, borosilicate glass, lead glass, aluminosilicate glass, glass-ceramics, and fiber glass), silica-free glasses (e.g., amorphous metals and polymers), and molecular liquids and molten salts. Glasses can contain additives that can modify e.g., the optical properties (e.g., transparency, color, refractivity etc.), conductive properties or other properties of the glass.

As used herein, “metal” refers to Li, Be, Na, Mg, Al, K, Ca, Sc, Ti, V, Cr, Mn, Fe, Co, Ni, Cu, Zn, Ga, Rb, Sr, Y, Zr, Nb, Mo, Tc, Ru, Rh, Pd, Ag, Cd, In, Sn, Cs, Ba, La, Ce, Pr, Nd, Pm, Sm, Eu, Gd, Tb, Dy, Ho, Er, Rm, Yb, Lu, Hf, W, Re, Os, Ir, Pt, Au, Hg, Tl, Pb, Bi, Po, Ra, Ac, Th, Pa, U, Np, Am, Cm, Bk, Cf, Es, Fm, Md, No, Lr, Rf, db, Sg, Bh, Hs, Mt, Ds, Rg, Cn, Nh, Fl, Mc, Lv, and combinations thereof. As used herein, “metalloid” refers to B, Si, Ge, As, Sb, Te, At, and combinations thereof. As used herein, “non-metal” refers to He, H, C, N, O, F, Ne, P, S, Cl, Ar, Se, Br, Kr, I, Xe, Rn, and combinations thereof.

As used herein, “fibrous material” refers to any bulk material composed of a plurality of fibers. The fibers the fibrous material can be composed of glass, biological polymers (e.g., proteins, polynucleotides), metals, metalloids, non-metals, carbon nanostructures, polymers, crystals, ceramics, metal alloys, and combinations thereof. The fibers can be formed of natural or synthetic materials. The fibrous material can form any usable form, such as a sheet, membrane, strip, tape, slide, fiber, mesh, and the like. The fibrous material can be a flexible, semi-flexible, or inflexible material. Generally, fibrous materials where the individual fibers are loosely coupled to or associated with each other will be more flexible than those where the individual fibers are more tightly coupled or associated with each other. Exemplary fibrous materials include, but are not limited to paper sheets, paper strips and paper tapes, polymeric membranes, fabrics, and fibrous glass membranes.

In some embodiments, all or one or more parts of the surface and/or the substrate can be hydrophilic. In some embodiments, all or one or more parts of the surface and/or the substrate can be hydrophobic. In some embodiments, all or one or more parts of the surface and/or substrate can be superhydrophobic. In some embodiments, patterns on the surface and/or substrate can be formed by specific placement of hydrophobic and/or hydrophilic materials. In some embodiments, such patterns can, without limitation, form features of the array and/or form conduits to provide sample, reactants, features, and the like to one or more regions of the array. As used herein, “hydrophilic”, refers to molecules which have a greater affinity for, and thus solubility in, water as compared to organic solvents. The hydrophilicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the water than in the organic solvent, then the molecule is considered hydrophilic. As used herein, “hydrophobic”, refers to molecules which have a greater affinity for, or solubility in an organic solvent as compared to water. The hydrophobicity of a compound can be quantified by measuring its partition coefficient between water (or a buffered aqueous solution) and a water-immiscible organic solvent, such as octanol, ethyl acetate, methylene chloride, or methyl tert-butyl ether. If after equilibration a greater concentration of the compound is present in the organic solvent than in the water, then the molecule is considered hydrophobic. In some embodiments, hydrophobic and hydrophilic regions can be formed by particular materials that are hydrophobic or hydrophilic or can be formed by changing the texture of a surface (e.g., by etching, scoring, etc.) such that the contact angle or other interaction of water or liquid with the surface is changed such that that region such that it is hydrophobic or hydrophilic.

In some embodiments, the suitable material can be a hydrophobic material. Suitable hydrophobic materials include, but are not limited to: acrylics (e.g., acrylic, acrylonitrile, acrylamide, and maleic anhydride polymers), polyamides and polyimides, carbonates (e.g., Bisphenol A-based carbonates), polydienes, polyesters, polyethers, polyfluorocarbons, polyolefins (e.g., polyethylene, polypropylene, and copolymers thereof), polystyrenes and copolymers thereof, polyvinyl acetals, polyvinyl chlorides and polyvinylidene chlorides, poly vinyl ethers and polyvinyl ketones, polyvinylpyridines and polyvinylpyrrolidones, Aculon's Transition Metal Complex coting, SLIPS coating material (Adaptive Surface Technologies), and any combination thereof.

In some embodiments, the suitable material can be composed of or include a superhydrophobic material. Suitable superhydrophobic materials include, but are not limited to manganese oxide polystyrene, zinc oxide polystyrene, precipitated calcium carbonate, carbon nanotubes, silica nano-coatings, fluorinated silanes, and flurophopolymer coatings. See e.g., Meng et al. 2008, The Journal of Physical Chemistry C. 112 (30): 11454-11458; Hu et al. 2009. Colloids and Surfaces A: Physicochemical and Engineering Aspects. 351 (1-3): 65-70; Lin et al., Colloids and Surfaces A: Physicochemical and Engineering Aspects. 421: 51-62; Das et al., RSC Advances. 4 (98): 54989-54997. doi:10.1039/C4RA10171E; Torun et al., 2018. Macromolecules. 51 (23): 10011-10020; Warsinger et al. 2015., Colloids and Surfaces A: Physicochemical and Engineering Aspects. 421: 51-62; Servi et al. 2017., Journal of Membrane Science. Elsevier BV. 523: 470-479

In some embodiments, the suitable material can be composed of or include a hydrophilic material. Hydrophilic materials include, but are not limited to, hydrophilic polymers such as poly(N-vinyl lactams), poly(vinylpyrrolidone), poly(ethylene oxide), poly(propylene oxide), polyacrylamides, cellulosics, methyl cellulose, polyanhydrides, polyacrylic acids, polyvinyl alcohols, polyvinyl ethers, alkylphenol ethoxylates, complex polyol monoesters, polyoxyethylene esters of oleic acid, polyoxyethylene sorbitan esters of oleic acid, and sorbitan esters of fatty acids; inorganic hydrophilic materials such as inorganic oxide, gold, zeolite, and diamond-like carbon; and surfactants such as Triton X-100, Tween, Sodium dodecyl sulfate (SDS), a.mmonium lauryl sulfate, alkyl sulfate salts, sodium lauryl ether sulfate (SLES), alkyl benzene sulfonate, soaps, fatty acid salts, cetyl trimethylammonium bromide (CTAB) a.k.a. hexadecyl trimethyl animonium bromide, alkyltrimethylanimonium salts, cetylpyridinium chloride (CPC), polyethoxylated tallow amine (POEA), benzalkonium chloride (BAC), benzethonium chloride (BZT), dodecyl betaine, dodecyl dimethylamine oxide, cocamidopropyl betaine, coco ampho glycinate alkyl poly(ethylene oxide), copolymers of poly(ethylene oxide) and poly(propylene oxide) (commercially called Poloxamers or Poloxamines), alkyl polyglucosides, fatty alcohols, cocamide MEA, cocamide DEA, cocamide TEA, Adhesives Research (AR) tape 90128, AR tape 90469, AR tape 90368, AR tape 90119, AR tape 92276, and AR tape 90741 (Adhesives Research, Inc., Glen Rock, Pa.). Examples of hydrophilic film include, but are not limited to, Vistex® and Visguard® films from (Film Specialties Inc., Hillsborough, N.J.), and Lexan HPFAF (GE Plastics, Pittsfield, Mass.). Other hydrophilic surfaces are available from Surmodics, Inc. (Eden Prairie, Minn.), Biocoat Inc. (Horsham, Pa.), Advanced Surface Technology (Billerica, Mass.), and Hydromer, Inc. (Branchburg, N.J.) and any combination thereof. Surfactants can be mixed with reaction polymers such as polyurethanes and epoxies to serve as a hydrophilic coating.

In some embodiments, the suitable material can be composed of or include a conductive and/or magnetic material. Conductive materials include, without limitation, metals, electrolytes, superconductors, semiconductors and some nonmetallic conductors such as graphite and conductive polymers. Magnetic materials include without limitation, any magnetic material including those that are ferromagnetic, paramagnetic and diamagnetic. In some embodiments, the magnetic material can include those that are electromagnetic (i.e., those materials that become magnetic or become a more powerful magnet when an electric current is applied to them). Exemplary magnetic materials include, but are not limited to, iron, nickel, cobalt, steel, rare earth metals (e.g. gadolinium, samarium, and neodymium), and combinations thereof.

In some embodiments, the suitable material can be composed or include an electric insulator material. Exemplary electric insulator materials include, but are not limited to, rubber, glass, oil, air, diamond, dry wood, dry cotton, plastic, fiberglass, porcelain, ceramics and quartz.

In some embodiments, the surface of the substrate is made from the same material as the substrate and is essentially integrated and indistinguishable from the substrate. In some embodiments, the surface is made from a different material as the substrate. In some embodiments the surface is essentially a coating, film, or layer present on at least part of or the entirety of the substrate and is thus readily distinguishable from the substrate.

### Array Features

As previously described the array can have one or more features. In some embodiments, one or more of the features can have sub-features. In some embodiments, the sub features themselves can form an array within the feature (or also referred to herein as a sub array).

The number of features can range from 1 to 100, 1,000, 10,000 or more. In some embodiments, the number of features can be 1, to 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, or 10000 or more.

The number of sub features can range from 1 to 100, 1,000, 10,000 or more. In some embodiments, the number of sub features can be 1 to/or 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900, 10000, 15000, 20000, 25000, 30000, 35000, 40000, 45000, 50000 or more.

In some embodiments the features and/or sub features can be wells (including but not limited to, microwells, nanowells, picowells, etc.), capillaries, microcapillaries, nanocapillaries, droplets, beads, oligonucleotides, polynucleotides, antibodies, affibodies, aptamers, polypeptide:polynucleotide complexes, gel forms, hydrogel forms, columns, matrices, and any permissible combinations thereof.

In some embodiments the features and/or sub features can hold a volume ranging from 1-1,000 pm³, nm³, μm³, cm³, mm³, or L³. In some embodiments, the wells, microwells, and/or nanowells capillaries, microcapillaries, nanocapillaries, and/or other areas formed on a surface of a substrate can hold a volume can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm³, nm³, μm³, cm³, mm³, or L³.

In some embodiments, one or more dimensions of the features and/or sub features (e.g. a length, a width, a height, a diameter, and the like) can range from about 1-1,000 pm, nm, μm, cm, or mm. In some embodiments, one or more dimensions of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the largest dimension of the features and/or sub features can range from 1-1,000 pm, nm, μm, cm, or mm. In some embodiments, the largest dimension of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm. In some embodiments, the smallest dimension of the features and/or sub features can range from about 1-1,000 pm, nm, μm, cm, or mm. In some embodiments, the smallest dimension of the surface can be about 1, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990 to/or about 1000 pm, nm, μm, cm, or mm.

In some embodiments the features can be any container, region, area, droplet, vessel, and the like capable of containing a volume of fluid. In some of such embodiments, the features can be wells, (including but not limited to, microwells, nanowells, picowells, etc.) capillaries, microcapillaries, nanocapillaries, and/or other areas formed on a surface of a substrate. The wells, microwells, and/or nanowells capillaries, microcapillaries, nanocapillaries, and/or other areas formed on a surface can be any regular or regular 2D or 3D shape. In some embodiments, all the wells, microwells, and/or nanowells capillaries, microcapillaries, nanocapillaries, and/or other areas formed on a surface are homogenous. In some embodiments, all the wells, microwells, and/or nanowells capillaries, microcapillaries, nanocapillaries, and/or other areas formed on a surface are heterogenous.

In some embodiments, the array can be configured for electrochemical polynucleotide detection, which is described in greater detail elsewhere herein. In some embodiments, the array can be a CMOS array. An exemplary CMOS array for electrochemical polynucleotide detection is described in U.S. Pat. No. 10,718,732, which can be adapted for use with the various embodiments described herein.

### Biomarker Amplification and Enrichment

In some embodiments, the biomarker to be detected can be amplified or enriched prior to detection.

### Biomarker Amplification

In certain example embodiments, detecting comprises amplification of one or more of the biomarkers. In some embodiments, the biomarker(s) is/are selectively amplified from a larger population of polynucleotides. The amplification may be performed with primers with one or more barcodes. Barcodes, such as those included on primers, adapters, or any other nucleic acid molecules described or contemplated herein, include short sequences of nucleotides (for example, DNA or RNA) used as an identifier for an associated molecule, such as a target molecule and/or target nucleic acid, or as an identifier of the source of an associated molecule, such as a cell-of-origin. A barcode may also refer to any unique, non-naturally occurring, nucleic acid sequence that may be used to identify the originating source of a nucleic acid fragment. Although it is not necessary to understand the mechanism of an invention, it is believed that the barcode sequence provides a high-quality individual read of a barcode associated with a single cell, a viral vector, labeling ligand (e.g., an aptamer), protein, shRNA, sgRNA or cDNA, such that multiple species can be sequenced together.

Barcoding may be performed based on any of the compositions or methods disclosed in International Patent Publication WO 2014047561 A1, Compositions and methods for labeling of agents, incorporated herein in its entirety. In certain embodiments barcoding uses an error correcting scheme (T. K. Moon, Error Correction Coding: Mathematical Methods and Algorithms (Wiley, New York, ed. 1, 2005)). Not being bound by a theory, amplified sequences from single cells can be sequenced together and resolved based on the barcode associated with each cell, sample, subject, pool, or a variety of other categories as desired depending on barcoding scheme applied.

In certain example embodiments, amplification can be performed using polymerase chain reaction (PCR), quantitative real-time PCR; reverse transcriptase PCR (RT-PCR); real-time PCR (rt PCR); real-time reverse transcriptase PCR (rt RT-PCR); nested PCR; strand displacement amplification; transcription-free isothermal amplification; ligase chain reaction amplification; gap filling ligase chain reaction amplification; coupled ligase detection and PCR; and NASBA™ RNA transcription-free amplification or other methods known in the art.

In certain example embodiments, amplification comprises nucleic-acid sequenced-based amplification (NASBA), recombinase polymerase amplification (RPA), loop-mediated isothermal amplification (LAMP), strand displacement amplification (SDA), helicase-dependent amplification (HDA), nicking enzyme amplification reaction (NEAR), multiple displacement amplification (MDA), rolling circle amplification (RCA), ligase chain reaction (LCR), ramification amplification method or (RAM), or a combination thereof.

In certain example embodiments, target RNAs and/or DNAs (e.g., a target replication-specific feature), may be amplified prior to performing a detection method described in greater detail elsewhere herein. Any suitable RNA or DNA amplification technique may be used. In certain example embodiments, the RNA or DNA amplification is an isothermal amplification. In certain example embodiments, the isothermal amplification may be nucleic-acid sequenced-based amplification (NASBA), recombinase polymerase amplification (RPA), loop-mediated isothermal amplification (LAMP), strand displacement amplification (SDA), helicase-dependent amplification (HDA), or nicking enzyme amplification reaction (NEAR). In certain example embodiments, non-isothermal amplification methods may be used which include, but are not limited to, PCR, multiple displacement amplification (MDA), rolling circle amplification (RCA), ligase chain reaction (LCR), or ramification amplification method (RAM). In certain embodiments, the amplification can utilize a transposase-based isothermal amplification method (see e.g. WO 2020/006049, which is incorporated by reference herein as if expressed in its entirety), nickase-based isothermal amplification method (see e.g. WO 2020/006067, which is incorporated by reference herein as if expressed in its entirety), or a helicase-based amplification method (see e.g. WO 2020/006036, which is incorporated by reference herein as if expressed in its entirety). In some embodiments, amplification is via LAMP. In some embodiments, amplification is via RPA.

In certain example embodiments, the RNA or DNA amplification is nucleic acid sequence-based amplification is NASBA, which is initiated with reverse transcription of target RNA by a sequence-specific reverse primer to create a RNA/DNA duplex. RNase H is then used to degrade the RNA template, allowing a forward primer containing a promoter, such as the T7 promoter, to bind and initiate elongation of the complementary strand, generating a double-stranded DNA product. The RNA polymerase promoter-mediated transcription of the DNA template then creates copies of the target RNA sequence. Importantly, each of the new target RNAs can be detected by a polynucleotide method described herein, thus increasing the sensitivity of the assay. The NASBA reaction has the additional advantage of being able to proceed under moderate isothermal conditions, for example at approximately 41° C., making it suitable for systems and devices deployed for early and direct detection in the field and far from clinical laboratories.

In certain other example embodiments, a recombinase polymerase amplification (RPA) reaction may be used to amplify the target nucleic acids. RPA reactions employ recombinases which are capable of pairing sequence-specific primers with homologous sequence in duplex DNA. If target DNA is present, DNA amplification is initiated and no other sample manipulation such as thermal cycling or chemical melting is required. The entire RPA amplification system is stable as a dried formulation and can be transported safely without refrigeration. RPA reactions may also be carried out at isothermal temperatures with an optimum reaction temperature of 37-42° C. The sequence specific primers are designed to amplify a sequence comprising the target nucleic acid sequence to be detected. In certain example embodiments, a RNA polymerase promoter, such as a T7 promoter, is added to one of the primers. This results in an amplified double-stranded DNA product comprising the target sequence and a RNA polymerase promoter. After, or during, the RPA reaction, a RNA polymerase is added that will produce RNA from the double-stranded DNA templates. The amplified target RNA can then in turn be detected by the polynucleotide detection system. In this way target DNA can be detected using the embodiments disclosed herein. RPA reactions can also be used to amplify target RNA. The target RNA is first converted to cDNA using a reverse transcriptase, followed by second strand DNA synthesis, at which point the RPA reaction proceeds as outlined above.

Accordingly, in certain example embodiments the systems disclosed herein may include amplification reagents. Different components or reagents useful for amplification of nucleic acids are described herein. For example, an amplification reagent as described herein may include a buffer, such as a Tris buffer. A Tris buffer may be used at any concentration appropriate for the desired application or use, for example including, but not limited to, a concentration of 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 25 mM, 50 mM, 75 mM, 1 M, or the like. One of skill in the art will be able to determine an appropriate concentration of a buffer such as Tris for use with the present invention.

A salt, such as magnesium chloride (MgCl₂), potassium chloride (KCl), or sodium chloride (NaCl), may be included in an amplification reaction, such as PCR, in order to improve the amplification of nucleic acid fragments. Although the salt concentration will depend on the particular reaction and application, in some embodiments, nucleic acid fragments of a particular size may produce optimum results at particular salt concentrations. Larger products may require altered salt concentrations, typically lower salt, in order to produce desired results, while amplification of smaller products may produce better results at higher salt concentrations. One of skill in the art will understand that the presence and/or concentration of a salt, along with alteration of salt concentrations, may alter the stringency of a biological or chemical reaction, and therefore any salt may be used that provides the appropriate conditions for a reaction of the present invention and as described herein.

Other components of a biological or chemical reaction may include a cell lysis component in order to break open or lyse a cell for analysis of the materials therein. A cell lysis component may include, but is not limited to, a detergent, a salt as described above, such as NaCl, KCl, ammonium sulfate [(NH₄)₂SO₄], or others. Detergents that may be appropriate for the invention may include Triton X-100, sodium dodecyl sulfate (SDS), CHAPS (3-[(3-cholamidopropyl)dimethylammonio]-1-propanesulfonate), ethyl trimethyl ammonium bromide, nonyl phenoxypolyethoxylethanol (NP-40). Concentrations of detergents may depend on the particular application and may be specific to the reaction in some cases. Amplification reactions may include dNTPs and nucleic acid primers used at any concentration appropriate for the invention, such as including, but not limited to, a concentration of 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 mM, 2 mM, 3 mM, 4 mM, 5 mM, 6 mM, 7 mM, 8 mM, 9 mM, 10 mM, 20 mM, 30 mM, 40 mM, 50 mM, 60 mM, 70 mM, 80 mM, 90 mM, 100 mM, 150 mM, 200 mM, 250 mM, 300 mM, 350 mM, 400 mM, 450 mM, 500 mM, or the like. Likewise, a polymerase useful in accordance with the invention may be any specific or general polymerase known in the art and useful or the invention, including Taq polymerase, Q5 polymerase, or the like.

In some embodiments, amplification reagents as described herein may be appropriate for use in hot-start amplification. Hot start amplification may be beneficial in some embodiments to reduce or eliminate dimerization of adaptor molecules or oligos, or to otherwise prevent unwanted amplification products or artifacts and obtain optimum amplification of the desired product. Many components described herein for use in amplification may also be used in hot-start amplification. In some embodiments, reagents or components appropriate for use with hot-start amplification may be used in place of one or more of the composition components as appropriate. For example, a polymerase or other reagent may be used that exhibits a desired activity at a particular temperature or other reaction condition. In some embodiments, reagents may be used that are designed or optimized for use in hot-start amplification, for example, a polymerase may be activated after transposition or after reaching a particular temperature. Such polymerases may be antibody-based or aptamer-based. Polymerases as described herein are known in the art. Examples of such reagents may include, but are not limited to, hot-start polymerases, hot-start dNTPs, and photo-caged dNTPs. Such reagents are known and available in the art. One of skill in the art will be able to determine the optimum temperatures as appropriate for individual reagents.

Amplification of nucleic acids may be performed using specific thermal cycle machinery or equipment and may be performed in single reactions or in bulk, such that any desired number of reactions may be performed simultaneously. In some embodiments, amplification may be performed using microfluidic or robotic devices, or may be performed using manual alteration in temperatures to achieve the desired amplification. In some embodiments, optimization may be performed to obtain the optimum reactions conditions for the particular application or materials. One of skill in the art will understand and be able to optimize reaction conditions to obtain sufficient amplification.

In certain embodiments, detection of DNA with the methods or systems of the invention requires transcription of the (amplified) DNA into RNA prior to detection.

### Nucleic Acid Biomarker Enrichment

In certain example embodiments, target RNA or DNA (such as a biomarker described elsewhere herein) may first be enriched prior to detection or amplification of the target RNA or DNA (such as a replication-specific feature). In certain example embodiments, this enrichment may be achieved by binding of the target nucleic acids by a CRISPR effector system, probes, capture molecules, etc. described elsewhere herein.

Current target-specific enrichment protocols require single-stranded nucleic acid prior to hybridization with probes. Among various advantages, the present embodiments can skip this step and enable direct targeting to double-stranded DNA (either partly or completely double-stranded). In addition, the embodiments disclosed herein are enzyme-driven targeting methods that offer faster kinetics and easier workflow allowing for isothermal enrichment. In certain example embodiments enrichment may take place between 20-37° C. In certain example embodiments, a set of guide RNAs to different target nucleic acids are used in a single assay, allowing for detection of multiple targets and/or multiple variants of a single target.

In certain example embodiments, a dead CRISPR effector protein may bind the target nucleic acid in solution and then subsequently be isolated from said solution. For example, the dead CRISPR effector protein bound to the target nucleic acid, may be isolated from the solution using an antibody or other molecule, such as an aptamer, that specifically binds the dead CRISPR effector protein.

In other example embodiments, the dead CRISPR effector protein or other capture molecule (polynucleotide, aptamer, or the like) may be bound to a solid substrate. A fixed substrate may refer to any material that is appropriate for or can be modified to be appropriate for the attachment of a polypeptide or a polynucleotide. Possible substrates include, but are not limited to, glass and modified functionalized glass, plastics (including acrylics, polystyrene and copolymers of styrene and other materials, polypropylene, polyethylene, polybutylene, polyurethanes, Teflon™, etc.), polysaccharides, nylon or nitrocellulose, ceramics, resins, silica or silica-based materials including silicon and modified silicon, carbon, metals, inorganic glasses, plastics, optical fiber bundles, and a variety of other polymers. In some embodiments, the solid support comprises a patterened surface suitable for immobilization of molecules in an ordered pattern. In certain embodiments a patterned surface refers to an arrangement of different regions in or on an exposed layer of a solid support. In some embodiments, the solid support comprises an array of wells or depressions in a surface. The composition and geometry of the solid support can vary with its use. In some embodiments, the solids support is a planar structure such as a slide, chip, microchip and/or array. As such, the surface of the substrate can be in the form of a planar layer. In some embodiments, the solid support comprises one or more surfaces of a flowcell. The term “flowcell” as used herein refers to a chamber comprising a solid surface across which one or more fluid reagent can be flowed. Example flowcells and related fluidic systems and detection platforms that can be readily used in the methods of the present disclosure are described, for example, in Bentley et al. Nature 456:53-59 (2008), WO 04/0918497, U.S. Pat. No. 7,057,026; WO 91/06678; WO 07/123744; U.S. Pat. Nos. 7,329,492; 7,211,414; 7,315,019; 7,405,281, and US 2008/0108082. In some embodiments, the solid support or its surface is non-planar, such as the inner or outer surface of a tube or vessel. In some embodiments, the solid support comprises microspheres or beads. “Microspheres,” “bead,” “particles,” are intended to mean within the context of a solid substrate to mean small discrete particles made of various material including, but not limited to, plastics, ceramics, glass, and polystyrene. In certain embodiments, the microspheres are magnetic microspheres or beads. In some embodiments, the beads may be porous. The bead sizes range from nanometers, e.g., 100 nm, to millimeters, e.g., 1 mm.

A sample containing, or suspected of containing, the target nucleic acids (e.g., a replication-specific feature) may then be exposed to the substrate to allow binding of the target nucleic acids to the bound dead CRISPR effector protein, probe, or other capture molecule. Non-target molecules may then be washed away. In certain example embodiments, the target nucleic acids may then be released from the CRISPR effector protein/guide RNA complex, probe, or other capture molecule for further detection using the methods disclosed herein. In certain example embodiments, the target nucleic acids may first be amplified as described herein.

In certain example embodiments, the CRISPR effector, probe, capture molecule and the like may be labeled with a binding tag. In certain example embodiments the CRISPR effector, probe, capture molecule and the like, may be chemically tagged. For example, the CRISPR effector, probe, capture molecule and the like, may be chemically biotinylated. In another example embodiment, a fusion may be created by adding additional sequence encoding a fusion to the CRISPR effector, probe, capture molecule and the like. One example of such a fusion is an AviTag™, which employs a highly targeted enzymatic conjugation of a single biotin on a unique 15 amino acid peptide tag. In certain embodiments, the CRISPR effector, probe, capture molecule and the like probe, may be labeled with a capture tag such as, but not limited to, GST, Myc, hemagglutinin (HA), green fluorescent protein (GFP), flag, His tag, TAP tag, and Fc tag. The binding tag, whether a fusion, chemical tag, or capture tag, may be used to either pull down the CRISPR effector system, probe, or other capture molecule or complex once it has bound a target nucleic acid or to fix the CRISPR effector system, CRISPR effector system, probe, or other capture molecule or complex on the solid substrate.

### Sequencing

In some embodiments detection includes sequencing the biomarker(s). Protein and nucleic acid sequencing methods and techniques are generally known in the art. Exemplary sequencing techniques are described herein.

### Protein Sequencing

Protein sequencing can involve determining the entire or partial amino acid sequence of a polypeptide (or protein), peptide. In some embodiments, the protein sequencing allows for determination and mapping post-translational modifications to the protein. In some embodiments, de novo protein sequencing is performed using mass-spectrometry (see e.g., Standing, K. G., 2001. Curr Opin Struct Biol. 13(5):595-601). In some embodiments, protein sequencing is performed using Edman degradation coupled with a mass spectrometry technique (see e.g., Myashita et al., 2021. PNAS. 98 (8) 4403-4408). Mass spectrometry methods are also described in greater detail elsewhere herein. In some embodiments, a next-generation protein sequencing method is employed (see e.g., Callahan et al., Trends Biochem Sci. 2020 January; 45(1): 76-89; Alfero et al., Trends Biochem Sci. 2020 January; 45(1): 76-89; and Tang, L., Nature Methods volume 15, page 997 (2018).

### Nucleic Acid Sequencing

Nucleic acid sequencing methods and techniques are generally known in the art and include low- and high-throughput methods. In some embodiments, the nucleic acids sequenced are DNA. In some embodiments, the nucleic acids sequenced are RNA. In some embodiments, both DNA and RNA are sequenced. In some embodiments, the nucleic acid sequencing method is or includes Sanger sequencing, capillary electrophoresis and fragment analysis, or a next generation sequencing technique. In some embodiments, the nucleic acid sequencing includes a method or technique that allows for determining post-translational modifications of the nucleic acid. In some embodiments, such a method includes bisulfite sequencing. Exemplary methods are described in e.g., Slatko et al., Curr Protoc Mol Biol. 2018 April; 122(1):e59. doi: 10.1002/cpmb.59; McCombie et al., Cold Spring Harb Perspect Med. 2019 Nov. 1; 9(11):a036798. doi: 10.1101/cshperspect.a036798; Gu et al., Annu Rev Pathol. 2019 Jan. 24; 14:319-338. doi: 10.1146/annurev-pathmechdis-012418-012751; Chen and Zhao. Hum Genomics. 2019 Aug. 1; 13(1):34. doi: 10.1186/s40246-019-0220-8; Kumar et al., Semin Thromb Hemost. 2019 October; 45(7):661-673. doi: 10.1055/s-0039-1688446; Levey and Boone. Cold Spring Harb Perspect Med. 2019 Jul. 1; 9(7):a025791. doi: 10.1101/cshperspect.a025791; Ravi et al., Methods Mol Biol. 2018; 1706:223-232. doi: 10.1007/978-1-4939-7471-9_12; Stark et al., Nat Rev Genet. 2019 November; 20(11):631-656. doi: 10.1038/s41576-019-0150-2; van Dijk et al., Trends Genet. 2018 September; 34(9):666-681; Hu et al., Hum Immunol. 2021 November; 82(11):801-811; Arora and Tollefsbol. Methods. 2021 March; 187:92-103. doi: 10.1016/j.ymeth.2020.09.008; Wrecycka et al., J Biotechnol. 2017 Nov. 10; 261:105-115; Gouil and Keniry. Essays Biochem. 2019 Dec. 20; 63(6):639-648; Li and Tollefsbol. Methods. 2021 March; 187:28-43; Gong et al., Small Methods. 2022 March; 6(3):e2101251. doi: 10.1002/smtd.202101251; Hrdlickova et al., Wiley Interdiscip Rev RNA. 2017 January; 8(1):10.1002/wrna.1364. doi: 10.1002/wrna.1364; Wang et al., Nat Rev Genet. 2009 January; 10(1):57-63. doi: 10.1038/nrg2484; Conesa et al., Genome Biol. 2016 Jan. 26; 17:13. doi: 10.1186/s13059-016-0881-8; Chen at al., Front Genet. 2019 Apr. 5; 10:317. doi: 10.3389/fgene.2019.00317; Leucken and Theis. Mol Syst Biol. 2019 Jun. 19; 15(6):e8746; Shendure et al., Nature Biotechnology. 26 (10): 1135-1145; Sanger et al., 1977. PNAS. 74 (12): 5463-5467, which are each incorporated by reference herein as if expressed in their entirety and can be adapted for use with the present disclosure.

In certain embodiments, detection involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; and Gierahn et al., “Seq-Well: portable, low-cost RNA sequencing of single cells at high throughput” Nature Methods 14, 395-398 (2017), all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; and International patent application number PCT/US2016/059239, published as WO2017164936 on Sep. 28, 2017, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

Further embodiments are illustrated in the following Examples which are given for illustrative purposes only and are not intended to limit the scope of the invention.

### EXAMPLES

Now having described the embodiments of the present disclosure, in general, the following Examples describe some additional embodiments of the present disclosure. While embodiments of the present disclosure are described in connection with the following examples and the corresponding text and figures, there is no intent to limit embodiments of the present disclosure to this description. On the contrary, the intent is to cover all alternatives, modifications, and equivalents included within the spirit and scope of embodiments of the present disclosure. The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

### Example 1

### Introduction

The spirochetal bacterium Borrelia burgdorferi is the primary agent of Lyme disease, a debilitating infection that is transmitted to humans by the bite of an infected Ixodes spp. of tick. Over the past 20 years in the United States, the incidence of Lyme disease has increased more than 2000 percent with an estimate of close to 476,000 patients diagnosed annually [1,2]. Increases in disease prevalence can be attributed to 1) geographical expansion of vector ticks; 2) higher pathogen carriage rates; 3) deforestation; 4) increase in physician awareness of Lyme disease; and 5) social behavior [3-5]. Given the number of complex variables contributing to the ascendency of Lyme disease, this pervasive problem is likely to continue for the foreseeable future.

Upon transmission from an infected tick to a human host, B. burgdorferi causes a biphasic infection with a variety of clinical manifestations [6]. Acute, localized infection results in vague symptoms including fever, myalgias and headaches with the notable exception of an erythema migrans ‘Bullseye-like’ rash [6,7]. If not promptly and properly treated, patients may go on to experience late-stage disease complications that affect many tissues and organ systems [8]. Lyme arthritis (LA)—proliferative synovitis of one or more large joints—is the most common late-stage manifestation of Lyme disease in the United States [3,9]. LA progression and symptom persistence are multi-factorial [10]. For example, adaptive autoantibodies to bacterial products correlate with LA severity [11-14] while genetic polymorphisms in humoral receptors make some more susceptible to adverse outcomes [15]. In addition, a recent discovery has implicated remnants of the bacterial cell envelope as a likely contributor to dis-ease pathology [16]. How these factors are connected, the consequences of their interplay, and other components that may contribute to the development and persistence of LA, are not known.

The typical Gram-negative cell envelope consists of an outer membrane (OM), an inner membrane (IM), and the periplasmic space in between. One essential component of the cell envelope—peptidoglycan—resides in the periplasm. Peptidoglycan (PG) is a gigadalton-sized biopolymer made up of rigid glycan strands composed of the repeating disaccharide N-acetyl-glucosamine (GlcNAc) and N-acetylmuramic acid (MurNAc), that are cross-linked by short peptides [17,18]. The primary function of PG is to protect the cell from bursting due to the high osmotic pressure created by the crowded bacterial cytoplasm [19]. PG position within the periplasm is critical to its protective properties. It is perhaps not surprising then that most diderms produce highly conserved proteins that precisely position PG relative to the other envelope components. Bacteria unable to produce peptidoglycan-associated proteins (PAPs), have severe defects in cell 1) growth; 2) division; 3) morphology; 4) communication; and 5) ability to withstand exogenous stress [20,21]. Interestingly, many of these seemingly structural cell-wall components moonlight as virulence factors that contribute to bacterial pathogenicity [20,22,23].

Relative to classical diderms, the B. burgdorferi cell envelope is riddled with anomalies. For example, despite being a diderm, B. burgdorferi does not produce Lipopolysaccharide [24-26]. The outer membrane (OM) contains host-derived cholesterol [27,28] and more than 100 different lipoproteins [29]. Flagella are not extruded from the envelope, but rather are contained entirely in the periplasmic space [30,31]. Cross-linking peptides in the PG cell-wall contain the atypical diamine L-Ornithine [16,32]. Further, the typical proteins which are associated with PG that provide both structural integrity and spatial continuity within the cell envelope, appear to be lacking. This Example at least describes the identification of a B. burgdorferi PAP, previously implicated as an immunomodulatory factor and determine its function in the cell envelope homeostasis. In addition, this Example at least provides evidence for a unique PG-PAP relationship that likely contributes to the pathogenic properties of B. burgdorferi PG.

### Results

### Identification of PAPS

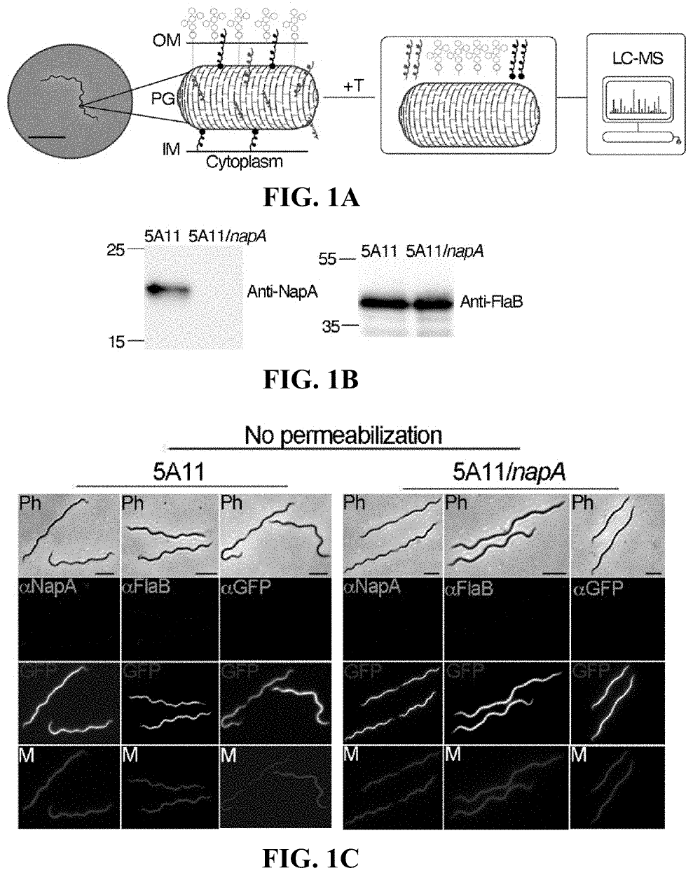

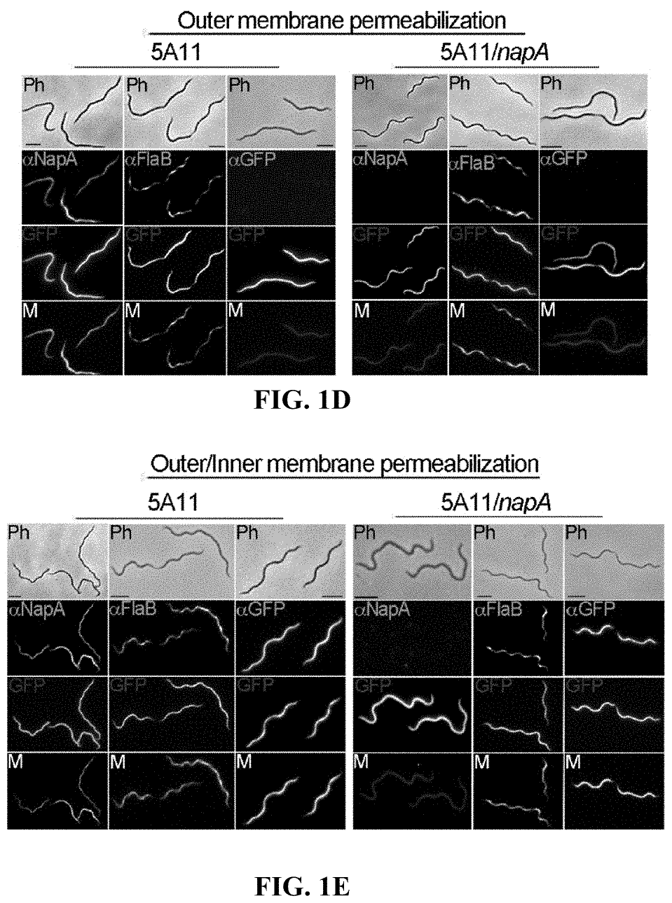

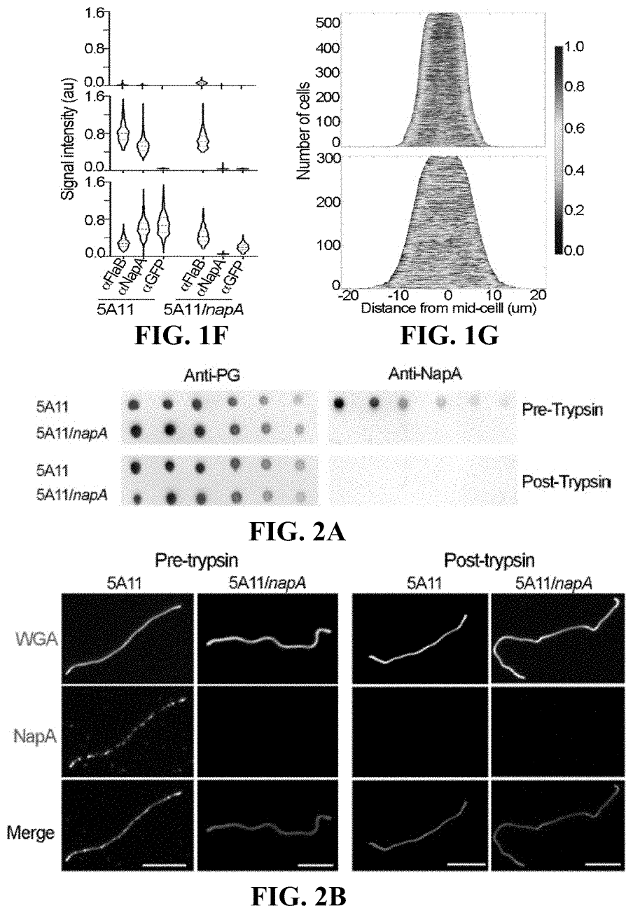

Despite their apparent paucity [24,25], it was hypothesized that B. burgdorferi does, indeed, produce PG-associated proteins (PAPs) that may be functionally akin to Braun's lipoprotein (i.e., play a structural role in PG and cell-wall support), but are not easily identifiable using standard in silico homology searches. To test Applicant's hypothesis without any a priori assumptions, Applicant purified B. burgdorferi PG using standard methods [16,33]. An initial purification step solubilizes most cellular components using 5% boiling sodium dodecyl sulfate (SDS). Following solubilization, PG as well as PG-associated material was collected. After washing to remove SDS, sacculi were treated with trypsin, which cleaves PAPs (FIG. 1A). Intact PG was removed from liberated PAP peptides and fragments were identified by LC-MS (FIG. 1A). Initial results yielded several candidate proteins but were contaminated with known cytoplasmic proteins (Table 1). In follow up experiments Applicant performed more rigorous sample processing, which included repeating the solubilization step with 5% boiling SDS. In these harsh sample prepara-tion procedures only two hits met the following criteria: 1) were present in all five biological replicates; 2) consistently had MASCOT scores>45; and 3) at least two unique peptides iden-tified in each experiment. Using these exclusion criteria BB0690 (Tables 1 and 2) was the top PAP candidate.

BB0690 has many names. Earlier studies identified BB0690 bioinformatically as a homo-logue of Dps (DNA binding protein from starved bacteria) and demonstrated that, like Dps, its production is further induced by stress [34-37]. Unlike Dps produced by most bacteria, BB0690 lacks the DNA-binding domain and does not bind DNA [34]. Curious, since the main function of Dps is to decorate DNA and protect heritable material from oxidative stress [37,38]. Subsequent studies demonstrated that BB0690 does play a role in oxidative stress by sequestering copper and iron metal ions, and thereby earned the name BicA (Borrelia iron copper binding protein A) [39]. Perhaps the most well studied phenomena associated with BB0690 is its ability to attract neutrophils and modulate innate immune responses [38,40-42] which precipitated the alternative moniker NapA (Neutrophil attracting protein A). Applicant contends that the basic function of BB0690 is not well understood. However, for simplicity, Applicant refers to BB0690 as NapA for this communication.

Sub-Cellular Localization of B. burgdorferi NapA

Despite the paradoxical role of NapA acting to protect B. burgdorferi against metal-stress

[34,39,43], but yet does not bind DNA [34], the sub-cellular localization of NapA has never been determined. To begin, Applicant first validated the specificity of polyclonal anti-NapA serum [35] raised in rabbits in two strains—a fully infectious derivative of the B31 type strain (5A11)

and a mutant strain in which the napA locus has been replaced by a kanamycin resistance cas-sette aphI1 (5A11/napA) [34]. Within the expected size range, anti-NapA yielded a single band in parent strain 5A11, which was absent in 5A11/napA (FIG. 1B). Comparative, whole genome sequencing results of 5A11 and 5A11/napA indicated that the resistance cassette, aphI1, completely replaced the napA locus in the mutant, but no additional mutations were present (Tables S3 and S4).

Upon strain and reagent validation, Applicant probed different cellular compartments for NapA using a modified immunofluorescence technique. Applicant reasoned that if NapA is directly associated with B. burgdorferi PG, then Applicant would expect to detect NapA in the periplasm. To ensure that Applicant was able to distinguish between possible periplasmic—from cytoplasmic-derived signal, Applicant co-transformed both the wild type 5A11 and 5A11/napA strains with a plasmid that constitutively produces GFP [44]. Applicant would expect that GFP would be exclusively localized to the cytoplasm. The periplasmic control consisted of the abundant flagella filament protein FlaB [31,45]. Both 5A11 and 5A11/napA strains were cultured, fixed with paraformaldehyde, and treated with buffer (no permeabilization), 50% methanol (OM permeabilization) [46], or detergent and PG-degrading lysozyme (OM/IM permeabilization). Each permeabilization method was validated by immunofluorescence using anti-FlaB (periplasm) and anti-GFP (cytoplasm). Regardless of strain, untreated fixed cells yielded no detectable signal when probed with any antibody, indicating that the fixation method did not compromise either spirochete membrane (FIGS. 1C and 1F, upper panel). In contrast, methanol treatment permeabilized the OM, as previously reported [46], resulting in robust anti-FlaB signal (FIGS. 1D and 1F, middle panel). Importantly, OM permeabilization did not result in loss of IM integrity, as indicated by background levels of signal intensity when probed with anti-GFP (FIGS. 1D and 1F, middle panel). As expected for a PAP, NapA was readily detectable in the periplasm, but only in 5A11 wild-type cells (FIG. 1D). Population level analysis of over 500 cells indicate that periplasmic NapA signal was intense, and comparable to the constitutively produced flagellar filament protein FlaB (FIG. 1F, middle panel). Not until fixed cells were completely permeabilized, were Applicant able to detect GFP with anti-GFP antibody (FIGS. 1E and 1F, lower panel). Under these conditions NapA can be readily detected as well, but demograph—single-cell, population-level signal intensity analysis, organized by cell-length—suggests cytoplasmic NapA signal is more sporadic than the uniform, periplasmic NapA signal (FIG. 1G). Applicant believed the latter could be attributed to the cytoplasmic permeabilization step, which also degrades some PG material with lysozyme treatment. Be that as it may, the limited permeabilization approach indicates that NapA originates in the cytoplasm but is readily detected in the B. burgdorferi periplasmic space.

NapA is Associated with the PG of B. burgdorferi

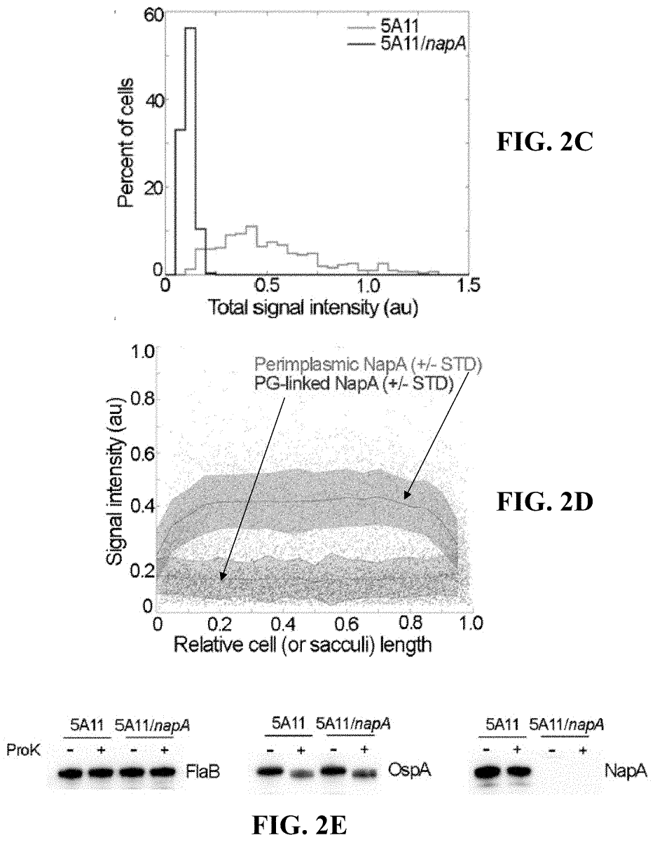

To determine if NapA is associated with PG, Applicant developed a strategy based on the concept of the screen (FIG. 1A). Parental and napA mutant strains were cultured to mid-log exponential growth, cellular components were solubilized with boiling SDS and insoluble material was collected. One half of the insoluble material was removed, and the remainder treated with tryp-sin, as is typical for PG purification. A dilution series of each sample was spotted on nitrocellu-lose and probed for NapA and PG. Dot blots demonstrated that PG was present in all samples tested (FIG. 2A), indicating that each sample was processed similarly and contained relatively equal amounts of cell-wall material. Much like PG, NapA signal was clearly present, and reduced with each dilution, but only in material purified from parental cells, prior to trypsin treatment (FIG. 2A). These data support the notion that, even after harsh treatment in boiling

detergent, NapA is associated with B. burgdorferi PG. To further assess their relationship, Applicant used immunofluorescence on separate biological replicate samples, prepared as described above. To circumvent anti-serum incompatibility issues (rabbit anti-PG/NapA), Applicant used Wheat Germ Agglutinin (WGA) conjugated to Alexa-350 to detect B. burgdorferi PG. WGA is known to bind GlcNAc—a ubiquitous PG sugar. Consistent with dot blot results, PG sacculi pre- and post-trypsin treatment were clearly present in relatively equal abundance for both parental and napA mutant strains (FIG. 2B). PG remained intact and WGA-PG derived signal was relatively uniform, with the possible exception of sacculi poles (FIG. 2B), suggesting that neither sample preparation nor the presence of NapA impacted PG signal (FIG. 2B). Prior to trypsin digestion, NapA appears to be scattered throughout the PG sacculus (FIG. 2B). Popula-tion level analysis of NapA signal, normalized by total sacculi area, was greater than 5-fold above background (FIG. 2C). While NapA signal appeared to display discrete patterning (FIG. 2B), population level assessment of relative position indicated that NapA is approximately equally distributed throughout PG sacculi (FIG. 2D). Overall NapA-PG signal, attained from purified sacculi, was about half that of periplasmic NapA signal (FIG. 1D), with clear depletion at the poles (FIG. 2D). The cause for the latter can be rationalized by the fact that intact cells have extended poles that are free of PG [33,45] and, by their association, NapA (FIG. 1G and FIG. 2D). The former is likely due to harsh purification steps. Taken together, the immunoblotting and immunofluorescence studies confirm that NapA is a PAP in the Lyme disease spirochete.

Some PAPs traverse the OM and can be detected on the bacterial surface [20]. Such localization would be noteworthy for a protein thought to influence immune cell chemotaxis [40-42]. To determine if NapA was surface-exposed, Applicant incubated live parental and napA mutant cells with Proteinase K, which would cleave surface-exposed proteins and alter their size by western blot. NapA was detected in wild type 5A11 cells, regardless of treatment, whereas surface-exposed OspA was readily cleaved and its mobility was clearly affected (FIG. 2E). The same samples and treatments were probed with anti-FlaB to confirm OM integrity during protease incubation. All samples contains FlaB and migrated similarly, confirming the sample preparation quality. Applicant concluded that NapA is located in the periplasm of B. burgdorferi, but is not surface-exposed.

### NapA Provides Structural and Physiological Integrity to the Cell-Wall

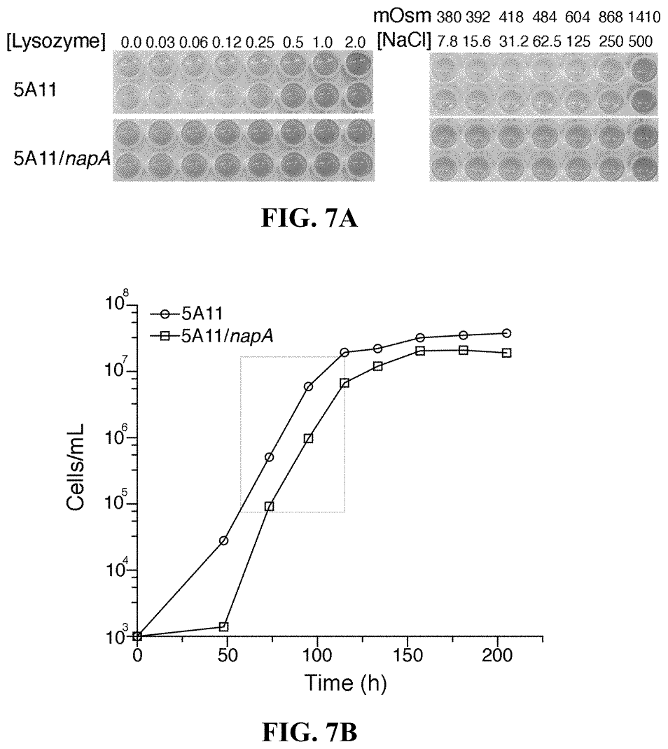

Since NapA is in the periplasm (FIG. 1A-1G) and decorates the PG sacculus of B. burgdorferi (FIG. 2A-2E), Applicant speculated that NapA-mediated protection from exogenous stress may be at the level of cell envelope integrity. If cell envelope integrity is compromised in a NapA deficient bacterium, then any cell-wall stress should produce a phenotype, not just oxidative stress [35,39]. To evaluate this, Applicant first incubated 5A11 and 5A11/napA cells with increasing amounts of NaCl and Lysozyme, which cause osmotic and PG specific stress, respectively. Applicant monitored microtiter plates for changes in pH—an indirect measurement of growth [47,48]. Wild-type cells were more than 8 times more resistant to NaCl-induced stress (FIG. 7A, right). Similarly, titrations of Lysozyme, which attacks the β 1-4 glycosidic linkage between PG glycan sugars GlcNAc and MurNAc ablated 5A11/napA growth at 5-6 times less enzyme than the wild-type bacteria (FIG. 7A, left).

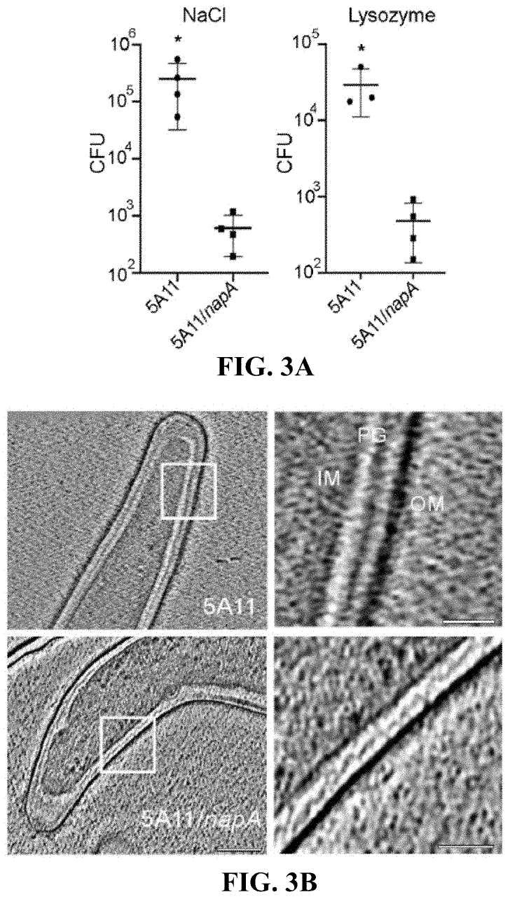

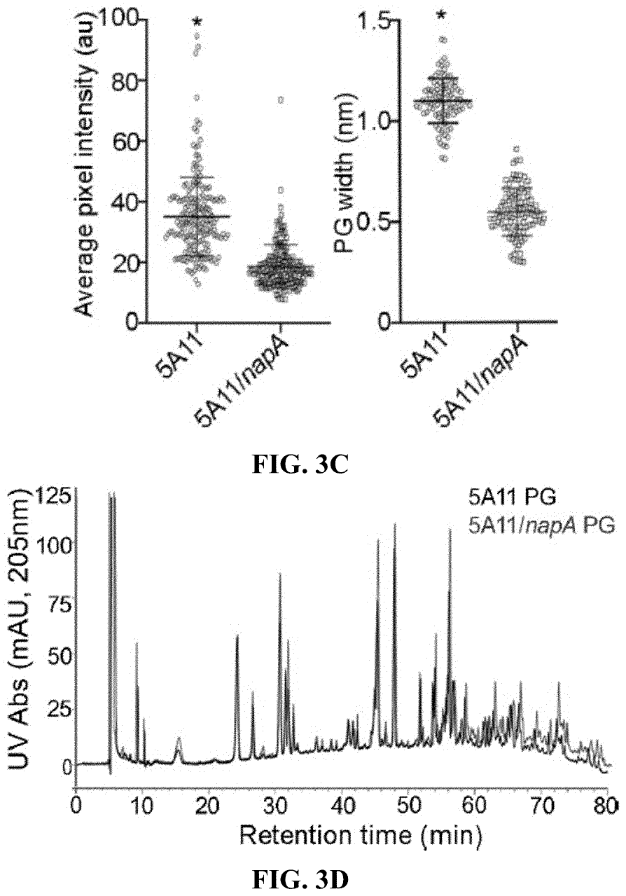

These data hint at a cell-wall defect in NapA deficient bacteria, however the microtiter plate assay also suggested a growth defect (FIG. 7A). Indeed, direct culture enumeration indicated that NapA deficient bacteria replicate 1.9 times slower than the parental strain (FIG. 7B). Since data presented in FIG. 7A were end-point measurements after 6 days, Applicant reasoned that the growth defect could account for the apparent differences in susceptibility to cell-wall stresses. To circumvent these issues, Applicant performed stress tests on both strains at a single, previously optimized concentration of NaCl and Lysozyme (FIG. 7A) for 18 hours in liquid broth. Each strain, and treatment, were then diluted in plating media lacking stress and colony forming units (CFUs) were determined. Wild-type CFUs were calculated after 3 weeks and, to compensate for growth defects (FIG. 7B), compared to results obtained from the napA mutant bacteria after 6 weeks. Even after accounting for growth rate defects, mutant bacteria were 2-3 logs lower in CFUs (FIG. 3A), indicating that NapA plays a basic role in cell envelope integrity and homeostasis. These data are consistent with earlier studies that NapA provides protection from exogenous stress [34,35,39]. However, Applicant surmise that NapA protects B. burgdorferi from all cell-wall stresses, potentially by reinforcing PG.

The distribution of NapA throughout the PG sacculi (FIGS. 2B and 2D) suggested that NapA may help bolster the PG, and that this association is essential to overall cell-wall integrity (FIG. 3A). Mechanistic insights were provided by comparative Cryo-Electron Microscopy (Cryo-EM) analysis of napA mutant and parental strains. Bacteria unable to produce NapA possessed compromised PG—appearing discontinuous, thinner, and more ruffled—relative to the thicker, more electron dense PG layer of the parental strain (FIG. 3B). Analysis of multiple Cryo-EM micrographs along the cell body from each strain demonstrated that both were true. The thickness of PG sacculi in mutant bacteria was, on average, roughly half (0.53) that of wild type cells (FIG. 3C). Integrated average PG pixel intensity values, normalized by sampling area, were also significantly lower in the napA mutant strain (FIG. 3C). It remained possible that the observed phenotype were the collective consequences of impairing PG biosynthesis. In other words, was NapA production in some way linked to PG synthesis, causing aberrations in cell-wall integrity? We addressed this possible scenario by purifying PG from the same number of cells, collected from both 5A11 and 5A11/napA strains. Pure PG was digested with mutanolysin, reduced, and separated by liquid chromatography. Comparative analysis of muropeptides profiles, attained from each strain, were nearly identical in terms of retention time and were negligibly different with respect to abundance (FIG. 3D), indicating the NapA production does not influence PG synthesis. Instead, these data support the role of NapA in cell envelope integrity and provide insights into the mechanism(s) by which it protects the cell from stress.

NapA and PG Fragments are Secreted in B. burgdorferi Outer Membrane Vesicles

Bacterial elongation requires PG synthesis. Newly synthesized PG multimers are incorporated into the existing structure, resulting in expansion, but at a cost. Each incorporation event requires that incisions are made to provide substrates for transglycosylation reactions. Most diderms typically recycle excised PG monomers back into the cytoplasm for reuse. B. burgdorferi lacks the transporters and enzymes necessary for PG recycling. The result—approximately 45% of B. burgdorferi PG is shed per generation from the periplasm into the extracellular environment [16]. How these PG fragments cross the outer membrane boundaries of the cell envelope is not known.

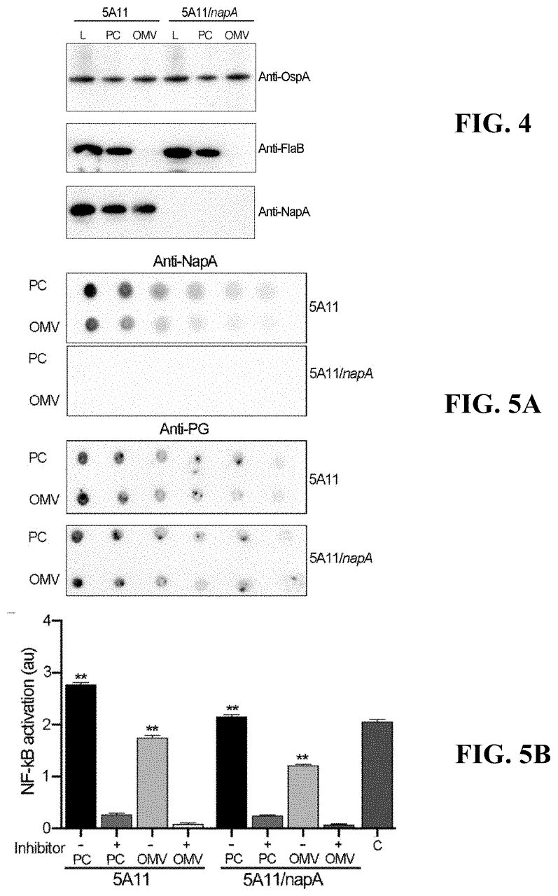

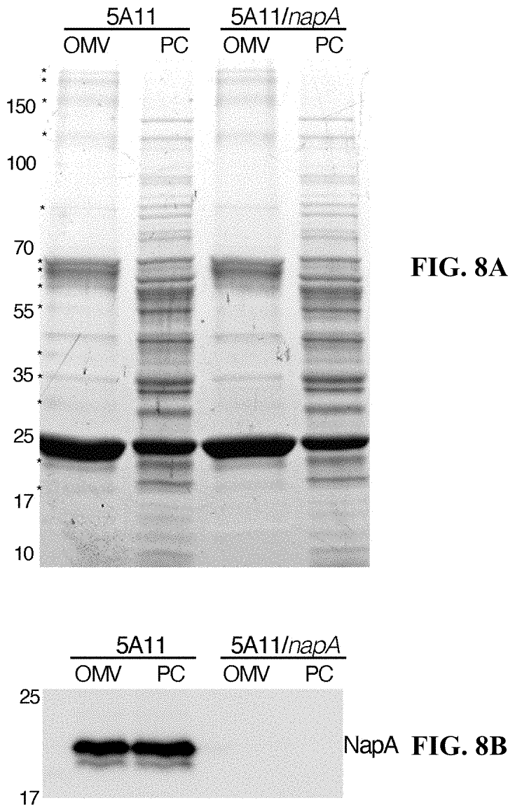

Applicant postulated that PG and, by extension, potentially NapA, could be released from the periplasm in Outer Membrane Vesicles (OMVs). B. burgdorferi produces OMVs, not only under stress, but also under regular homeostatic conditions [49], which support PG release [16]. To extend upon these findings and query OMVs for specific occupants, Applicant prepared OMVs and protoplasmic cylinder (PC) fractions from wild-type and napA mutant bacteria as previously described [49] and compared Applicant's fractions (FIG. 8), by immunoblotting to a portion of the input (lysate, L). Fractions from each strain contained OspA (FIG. 4), a well characterized OM protein known to be released in OMVs [49,50]. In contrast, FlaB—an abundant PC protein—was undetectable in the OMV preparations (FIG. 4). Probing each fraction for NapA yielded similar results to OspA, indicating that the full-length protein was indeed in B. burgdorferi OMVs isolated from wild type cells (FIG. 4).

Given the NapA-PG association, Applicant reasoned that released muropeptides and/or fragments of polymeric PG may also be included in OMVs. Given the large distribution of potential PG sizes, Applicant opted for dot blot analysis of each fraction and co-immunoblotting with anti-PG and anti-NapA. PG could be detected in the OMV fraction in both parental and napA mutant preparations (FIG. 5A), indicating that NapA is not required for PG to be present in OMVs. Applicant further confirmed that B. burgdorferi OMVs contain PG by a ligand-receptor reporter assay. PC and OMV fractions were incubated with a hNOD2 receptor reporter cell line, which, when exposed to PG containing Muramyl-L-Alanine-D-Glutamine (MDP), activates the secretion of alkaline phosphatase. To control for non-specific activation, Applicant included the inhibitor gefitinib, which acts on the adaptor protein RIP2, downstream of NOD2 signaling [16,51]. Both fractions from each strain resulted in significant hNOD2 activation (FIG. 5B), which was reduced 5-6-fold when inhibitor was added (FIG. 5B). Applicant notes that OMV contents were capable of activating a cytoplasmic receptor which indicates that 1) B. burgdorferi OMVs lysed during the experiment; 2) uptake occurs via phagocytosis; or 3) they are capable of fusing with eukaryotic membranes and expelling their contents, as reported for other bacteria [28,52].

NapA-Associated PG Acts as a Molecular Beacon, Augmenting the Immunomodulatory Properties of the B. burgdorferi Cell Wall

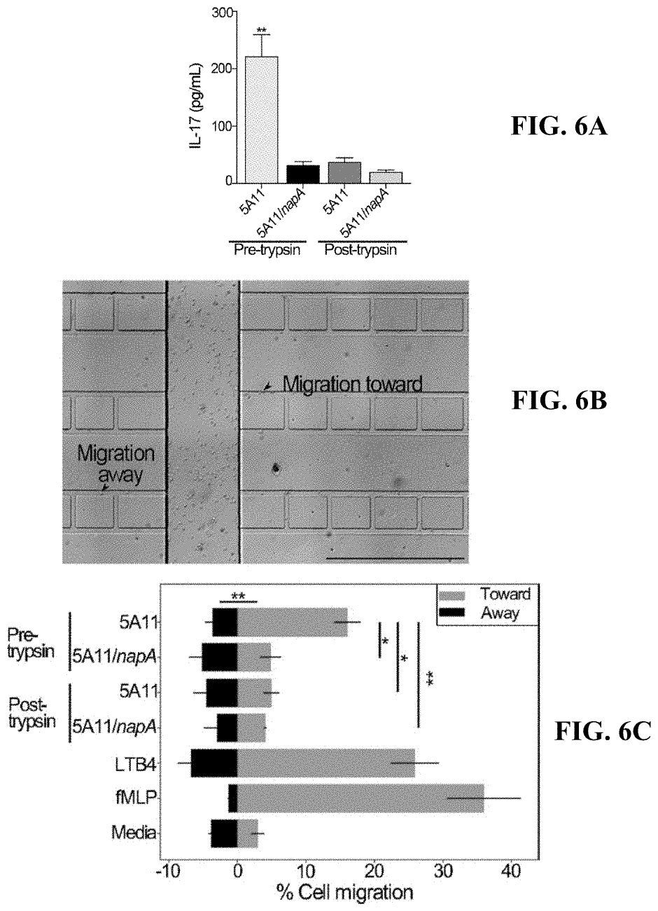

B. burgdorferi PG was recently shown to be a persistent antigen in the synovium of LA patients and is capable of inducing both inflammation and arthritis [16]. These studies were performed using purified B. burgdorferi PG, which includes a trypsin digestion step to cleave any linked proteins. Since PG is associated with NapA in its natural biological state, Applicant questioned whether the combination may augment the inflammatory response. Here, Applicant focused on IL-17 since 1) B. burgdorferi PG only modestly increased IL-17 secretion [16]; 2) IL-17 is markedly over-represented in LA patients [16,53,54]; and 3) previous studies have found that recombinant NapA can stimulate an TH1/TH17 response [40,42]. Using OMVs containing NapA-PG is complicated by package contents and casing. Instead, Applicant used the same pre- and post-trypsin treated PG samples as above, prepared from wild-type and mutant napA bacteria. Relative to napA mutant derived PG preparations, wild-type PG caused human PBMCs to secrete ˜9-fold more IL-17 (FIG. 6A), which highlights two important points: 1) The NapA-PG association has immunological consequences and 2) while it is possible that other proteins are associated with B. burgdorferi PG (Tables 1 and 2), NapA alone seems sufficient to augment the PG-induced IL-17 response (FIG. 6A).

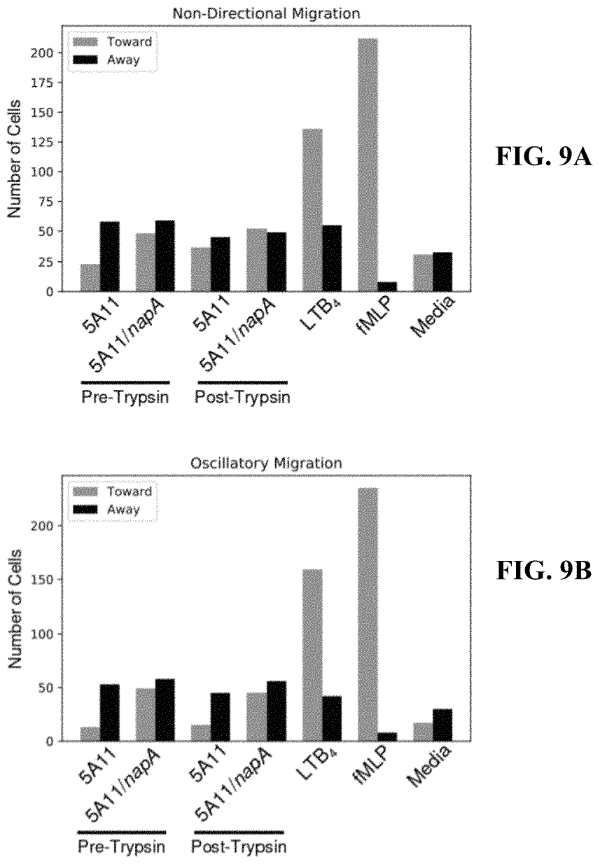

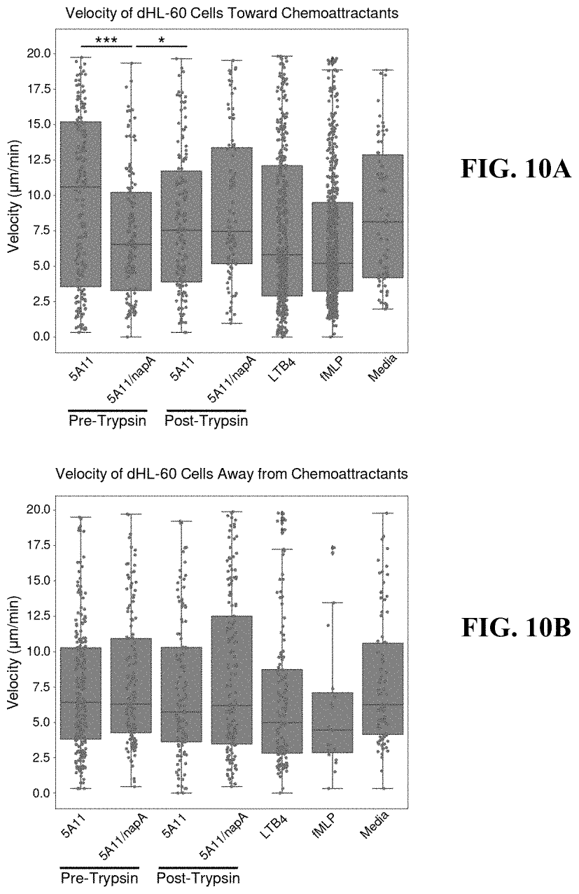

Since NapA-PG produced higher levels of IL-17, it may also act as a molecular beacon for neutrophils, naturally. To determine the chemoattractant capabilities of NapA-PG, Applicant per-formed a comparative study using real-time neutrophil tracking in a microfluidics chamber. In this system, neutrophils flow into a central chamber that is flanked by reservoirs on each side (FIG. 6B). Migratory bait is added to one reservoir and compared to the adjacent media-containing reservoir. Migration towards a potential stimulus was monitored by phase-contrast and epifluorescent microscopy for 5 hours. Percent migration was determined by the number of cells that reached a flanking reservoir. The only PG bait that acted as a significant chemoattractant was NapA-associated PG (16.03±1.93%) (FIG. 6C and S1-S7 Movies in Davis et al., 2021. PloS Pathog 17(5): e1009546, which are incorporated herein by reference as if expressed in their entireties herein); similar to that of known attractants LTB4 (25.89±3.52%) and fMLP (35.94±5.42%) [55]. None of the other preparations caused significant attraction or repulsion (FIG. 6C). Cells migrating toward NapA-associated PG also showed more directional migration—less cells migrated within cell mazes and displayed oscillatory migration (Table 5 and FIG. 9A-9B). Moreover, cells migrated toward NapA-associated PG with higher velocity (10.94±4.79 μm/min), relative to cells migrating toward other preparations (Table 5 and FIG. 10A-10B). Since data presented are the combined results of three biological replicates, and the only difference between pre-trypsin treated 5A11 PG and pre-trypsin treated napA/5A11 PG is the presence of NapA, the data suggest that NapA is both necessary and sufficient to cause neutrophil migration toward B. burgdorferi PG.

### Discussion

This Example at least provides evidence for a PAP in B. burgdorferi. Applicant reports that NapA exists in the periplasm but is not surface exposed (FIG. 1A-1G and FIG. 2A-2E). Molecular and cellular studies demonstrate a NapA-PG interaction, and that this association is important in stabilizing the B. burgdorferi cell envelope (FIG. 2A-2E and FIG. 3A-3D). NapA-PG is not only important for physical and physiological homeostasis, but also the nature of the interaction has pathogenic consequences resulting in increased IL-17 production and neutrophil attraction (FIG. 6A-6C). Here Applicant discusses the findings in the context of spirochete biology, Lyme disease, pathogenesis, and bacterial evolution.

Classical PAPs, produced by most bacteria, play a basic physiological role in cell envelope homeostasis. These proteins are often abundant and function to situate the PG layer at an appropriate distance from the IM and/or OM, often through lipidation. In this sense, NapA is atypical in that structural [41] and in silico analysis of the N-terminal region lack evidence for a lipidation site [29,56,57]. Instead of acting as a structuring scaffold to maintain PG position within the periplasm, Applicant favor an alternative mode of cell envelope protection whereby NapA decorates the PG (FIG. 2A-2E), provides continuity during turnover (FIG. 3A-3D), and both sequesters reactive species [34-36,39] and other exogeneous stress (FIG. 3A-3D). Recent studies in pathogenic Leptospira discovered a novel PG binding lipoprotein LipL21, which functions to bolster the PG while also acting to protect the cell from NOD1 and NOD2 detection [58], highlighting the dual function of seemingly pure structural proteins [17,20,23].

The natural life cycle of B. burgdorferi is complex and involves establishing residency in very different hosts, including the tick vector and dozens of potential vertebrate hosts [26]. Earlier studies have shown that napA is dispensable for mouse infection but required for tick survival [34]. Bacteria unable to produce NapA are more susceptible to PG-specific stress (FIG. 3A-3D). With the exception of host blood, it is not clear what stressors would be present in the tick mid-gut or how NapA ameliorates the osmoprotective properties of PG. Ixodes scapularis, however, does produce the B. burgdorferi PG-specific hydrolyzing enzyme Dae2 [59,60] in addition to lysozyme [61], which could function more effectively in the absence of NapA-linked PG (FIG. 3A-3D).

Clearly, much remains to be determined in lieu of these findings. For instance, the nature of the NapA-PG association is not known. PAPs bind their PG substrate through covalent and non-covalent interactions [62]. While Applicant cannot exclude either possibility, Applicant observed less NapA signal in purified PG relative to periplasmic-derived NapA signal (FIG. 2D); the latter is boiled for hours in 5% SDS. This suggests non-covalent interaction(s). Furthermore, B. burgdorferi NapA lacks the classical export signal sequence consistent with Sec-mediated secretion [29,57]. Others have found instances in which proteins are secreted from the cytoplasm through unknown mechanism(s), both in B. burgdorferi [63-66] and in many other bacterial phyla [67]. Applicant speculated that this could occur in conjunction with flagellum assembly through the dedicated Type 3 Secretion System, a system that lacks a consensus signal sequence [68] and is capable of exporting non-flagellar components associated with cell envelope homeostasis and virulence [69-73]. Alternatively, a yet to be defined system that is functionally analogous to the twin arginine transporter [74], which secretes folded proteins [75], but recognizes a different signal sequence, could be present in the B. burgdorferi genome, although not easily identified using standard bioinformatics. Far too often bioinformatics have failed to correctly assign seemingly conserved biological function to hypothetical proteins in this unusual genus [76-78]. These anomalies notwithstanding, NapA is readily detected in the periplasm (FIG. 1A-1G), associated with PG (FIG. 2A-2E and FIG. 7A-7B and Table S), and abundant in OMVs (FIG. 4 and FIG. 5A-5B). Applicant notes that others have corroborated the latter (personal communication, Wolfram Zückert).

Cell elongation requires both PG anabolism and catabolism. Excised muropeptides accumulate outside the cell and are involved in the pathogenesis of LA [16]. Until now, there has been no mechanism to explain how released PG crosses the B. burgdorferi OM. Here, Applicant shows that one route of PG release is through OMVs (FIG. 5A-5B). Based on the cellular reporter assay, OMV contents can end up inside eukaryotic cells (FIG. 5B). Several mechanisms have been pro-posed, including endocytosis and membrane fusion [52]. Two-way lipid exchange has been shown to occur following the internalization of B. burgdorferi OMVs [28], which supports the notion that OMVs may also be used for exchange of periplasmic contents such as NapA and other potentially pathogenic material. Of course, it is also possible the OMVs lyse, spilling their contents into the extracellular space of host systems. Regardless of the possible mechanism, NapA-linked PG augments the helper T cell response caused by PG alone, inducing higher levels of IL-17 (FIG. 6A). These findings are in line with studies using rNapA, which has been implicated in LA [40,42]. While other PAPs are likely (Table 2), these effects can likely be attributed to NapA-PG.

Neutrophils are akin to a platoon on the front lines—controlling the environment, initiating a response, and recruiting backup. During the initial stages of infection, neutrophils are recruited to the site of the tick bite; phagocytize B. burgdorferi; utilize lethal enzymes; and destroy bacterial cells using neutrophil extracellular traps (NETs) [79-81]. This initial attraction may be due to NapA-linked PG released from B. burgdorferi during growth or death at the site of inoculation. The latter may be a diversionary tactic whereby healthy bacteria are able to disperse to other parts of the body and cause more severe symptomology. In these later stages several different organs systems are involved, including the joints, heart, and central nervous system [9]. B. burgdorferi PG lingers in humans suffering from Lyme arthritis and trypsin treated PG can induce arthritis in the mouse model [16]. Therefore, the coordinated effect of both NapA and PG within the synovial tissue during later stages could exacerbate arthritis severity through the chemotactic properties of NapA-PG (FIG. 6C). Taken together, these findings implicate that a structural protein moonlights as a molecular beacon for immune cells and attracts them to an abundant inflammatory molecule.

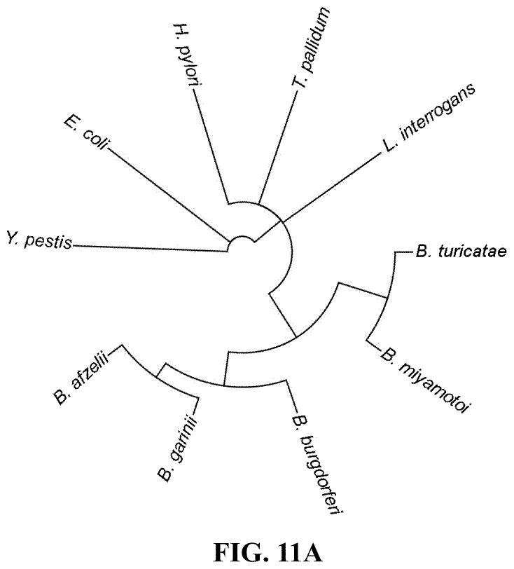

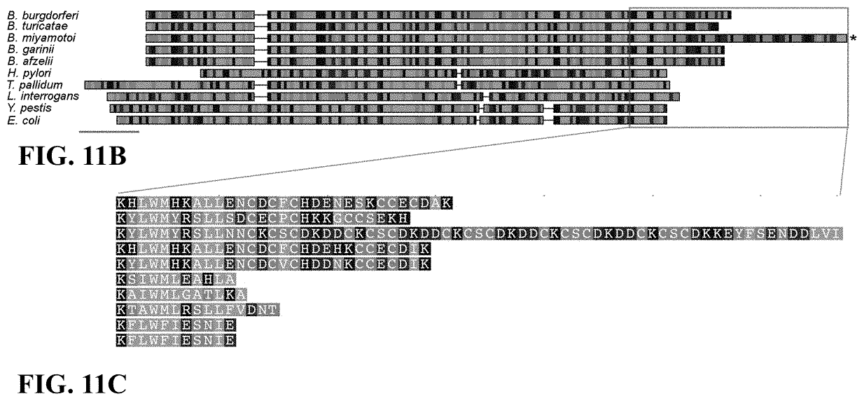

Dps homologues are produced by virtually all bacteria [82]. NapA shares structural and amino acid sequence similarities to some of the well-studied Dps homologues [37,41] (FIG. 11A-11C). There are, however, notable differences which may extend to other proteins which may have evolved to perform altered functions. 1) Dps production is highly upregulated in stressed conditions, and 2) acts by shielding DNA for oxidative damage, a ubiquitous function that appears

to be highly conserved across diverse taxa [36]. B. burgdorferi NapA, on the other hand, does not bind DNA [34] and its role in cellular homeostasis is in the periplasm (FIGS. 1A-1G, 2A-2E, and 3A-3D). NapA production does appear to increase under oxidative stress [35], but others have argued that basal production is considerable in culture ([34] and FIGS. 1D and 1F) and increased NapA expression by metal stress is negligible [83]. These findings are in line with the latter—Applicant detected considerable NapA under exponential growth (FIG. 1A-1G), which would make sense for a structural protein. At the amino acid level, B. burgdorferi NapA has two distinct features that separate it from other Dps homologues. First, much like H. pylori, B. burgdorferi NapA has a truncated N-terminus that lacks the Lysine-rich residues implicated in DNA binding [34,84,85]. Unlike H. pylori NapA, the B. burgdorferi homologue has an extended C-terminus, which appears to be a unique to Borreliae (FIG. 11B-11C). The biological consequences of these differences remains to be determined. Regardless of the differences between Dps homologues, these findings highlight the ingenuity of bacteria in which a protein can change from a mechanistic standpoint, while maintaining the same basic biological function of protecting the cell from exogenous stress.

### Materials and Methods

### Bacterial Strains, Eukaryotic Cells, and Growth Conditions

Both B. burgdorferi strains used in this study were generously provided by Frank Gheradini (NIH). A laboratory clone of the B. burgdorferi B31 type strain, termed 5A11 [86] served as the wild-type parental control. The napA mutant was produced in the same 5A11 background and was created by allelic replacement as described previously [34]. To produce low-level, constitutive GFP expressing napA and 5A11 strains, Applicant first created a promoter fusion construct between bb0826 and monomeric super-folder gfp fusion construct [44] using compatible SacI and BamHI. The resulting plasmid (pBLJ516) was transformed [87] into each strain, and clones were selected by micro-plate dilution with gentamicin (40 ug/mL), followed by fluorescence microscopy screening (see below). All B. burgdorferi cultures were propagated and maintained in Barbour-Stoenner-Kelly II (BSK-II) medium containing 6% rabbit serum at 37° C. under 5% CO₂.

Fresh, mixed donor human peripheral blood mononuclear cells (PBMCs) (Zen-Bio) were re-suspended in PBMC media (Zen-Bio) overnight prior to stimulations. Human NOD2 reporter cells, which were used to detect PG in OMVs (see below), were purchased from Invi-vogen and cultured as recommended by the manufacturer. Human promyelocytic leukemia cells (HL-60 CCL-240, American Type Culture Collection ATCC, Manassas, Va.) were cul-tured in complete media comprising of Iscove's Modified Dulbecco's Medium (IMDM, ATCC, Manassas, Va.) supplemented with 10% fetal bovine serum (FBS, ATCC, Manassas, Va.) at 37° C. in 5% CO2, according to ATCC instructions. HL-60 cells were differentiated into a neutrophil-like state with 1.5% dimethyl sulfoxide (DMSO, Sigma-Aldrich, St. Louis, Mo.) to 1.5×10⁵ cells/mL for five days. Differentiated HL-60 cells (dHL-60 cells) were stained with Hoechst solution [20 mM] for 10 minutes (Thermo Fisher Scientific, Waltham, Mass.) at 37° C. and 5% CO₂ and spun down and re-suspended into a concentration of 5.0×107 cells/mL immediately before use in the migration assay.

B. burgdorferi DNA Purification and Genome Sequencing

Low-passage, 40 mL cultures of each strain, were propagated to late-log (108 cells/mL) and harvested. After washing bacterial pellets three times with PBS, cells were lysed by sonication and DNA was extracted by standard phenol:chloroform methods. Crude DNA extracts were then purified using Zymo Research (Irvine, Calif.) genomic DNA purification kit.

Whole genomic sequencing was performed by Microbial Genome Sequencing Center (MiGS, Pittsburgh, Pa.), who provided >450× coverage for each sample. Unicycler was used to process and assemble all sequence data. Results were compared to the published type strain [24] and parent of the wild type derivative (5A11) used in these studies.

### Peptidoglycan Purification and Analysis

B. burgdorferi PG was purified as described previously [16] but was typically from 1 L of culture. Briefly, cells were harvested at 3,500×g, washed three times with PBS, and resuspended in PBS. Cell suspensions were added dropwise to 10% of boiling SDS. The final concentration of SDS 5%. After boiling for 1-hour, insoluble material was collected by ultra-centrifugation at 275,000×g for 1 hour and washed five times with 20 mL of 35° C. water. PG was resuspended in PBS and treated with 1000 U Benzonase Nuclease (Sigma-Aldrich) for 4 hours at 37° C., followed by overnight digestion with 300 μg/mL chymotrypsin (Sigma-Aldrich) at the same temperature. SDS (1%, final concentration) was added to each and boiled briefly. Insoluble material was once again harvested as described above and washed 3 times. For comparative studies that included PAPs, samples were processed the same as above, but split such that only half of the material was treated with 300 μg/mL chymotrypsin. In immunological an chemoattractant studies, after processing as described above, each sample was digested with Mutanolysin (10,000 U/mL) at 37° C. overnight. After removing undigested material by centrifugation, supernatants containing muropeptides were passed through a YM-10 filter and flow through dried. The concentration of the purified B. burgdorferi PG was determined using dry weight. B. burgdorferi PG was re-suspended in phosphate buffered saline (Thermo-Fisher) prior to use. Applicant notes that for all experiments in which PG preparations were treated with tryp-sin for PAP validation (FIGS. 2A-2E, 5A-5B, and 6A-6C) chymotrypsin was used, but for simplicity Applicant referred to digestions in figures a ‘trypsin+or trypsin treated’.

For muropeptide analysis bacterial density was determined from 500 mL cultures and standardized such that PG was purified from an equal number of cells (2.5×1010 cells). PG purification and mutanolysin digestion occurred as described above. After centrifugation (21,000×g for 30 minutes) to remove undigested material, supernatants were lyophilized. The resulting muropeptides were reduced with borohydride and analyzed as described previously [16].

### Identification of PAPs

To identify protein(s) associated with B. burgdorferi PG Applicant performed the same crude extraction procedure described above, with the exception that insoluble PG sacculi were treated with Mass Spectroscopy grade Trypsin (Sigma-Aldrich), instead of chymotrypsin. Peptides released by protease treatments were desalted utilizing 100 μl C18 Bond Elut OMIX (Agilent) SPE tips following the manufacturer's recommended protocol. Eluents were concentrated to dryness using a centrifugal vacuum concentrator. Peptides were reconstituted in 40 μl solvent A (98:2 LC-MS grade water: LC-MS grade acetonitrile supplemented with 0.1% (v/v) formic acid) by sonication. LC-MS grade solvents were obtained from Fisher Scientific. Aliquots (10 μL) were analyzed by liquid chromatography tandem mass spectrometry in data-dependent, positive ion mode, using an Orbitrap Fusion Lumos coupled to an Easy nLC1200 UPLC/autosampler (Thermo Scientific). Sample was loaded onto an C18 EASY-Spray HPLC analytical column (50 μm ID×15 cm, 2 μm particle size with 100 and 0.1 nm pore size, Thermo Scientific), and peptides were eluted from the system at a flow rate of 300 nl/min with a 110-minute gradient from 98% solvent A to 55% solvent A. Solvent B was 20:80 LC-MS grade water: LC-MS grade acetonitrile supplemented with 0.1% (v/v) formic acid. The analytical column was maintained at 55° C. and the ion transfer tube at 275° C. Electrospray voltage was set to 3000 V and the RF lens set to 30%. The MS1 scan utilized the orbitrap set to 120,000 resolution (m/z 200) over the m/z range of 500 to 1000 with an AGC target of 4e5, a maximum injection time of 50 msec in profile positive ion mode. Peaks exhibiting an isotopic envelope resembling a peptide with a charge of +2 to +5 and an intensity of at least 2e4 were subjected to MS2. MS2 utilized quadrupole isolation of ±1.4 Da, the orbitrap detector set to 15,000 resolution (m/z 200) and stepped HCD of 29-31% with the first mass of the MS2 scan set to 150 and a default charge state of +3. The AGC target was 1e5 with a maximum injection time of 200 msec in centroid positive ion mode. Dynamic exclusion prevented MS2 on the same peak for 15 seconds. Peptides were identified using Proteome Discoverer 2.2 (Thermo Scientific) using both Sequest HT and Mascot search engines. The data was searched against the B. burgdorferi reference proteome down-loaded from UniProt and concatenated with a database containing common lab contaminant proteins. All peptides for trypsin digests were expected to be fully-specific for trypsin digestion with the possibility of up to two missed cleavages. All peptides for chymotrypsin digests were expected to be fully-specific for trypsin digestion with the possibility of up to three missed cleavages. MS1 tolerance was set to ±10 ppm and MS2 tolerance was set to ±0.1 Da. Oxidation of methionine, deamidation of asparagine and glutamine, acetylation of the protein N-terminus and formation of pyroglutamate from glutamine when at the N-terminus of a peptide were set as variable modifications.

In secondary experiments, Applicant performed an additional solubilization step, prior to LC-MS identification of released peptides. Briefly, after solubilizing cellular material with 5% boiling SDS for 1 hour, PG was harvested by centrifugation, as described above. After washing 3 times with 20 mL of ˜50° C. ultra-pure water, PG was re-extracted with 5% boiling SDS for an additional hour and allowed to cool to room temperature overnight. The next day, material was re-heated to 80° C. for 30 minutes, centrifugation and washed, as described above, prior to peptide identification.

### Cellular Fractionation

Periplasmic fractions from napA/5A11 and 5A11 parental cultures were isolated essentially as previously described [49]. Briefly, one liter of each strain was cultured in BSKII supplemented with 6% rabbit serum to a final density of 2.5×107 cells/mL. To create a crude cell lysate, 40 mL of each culture was separated from the 1-liter bulk culture and processed separately. Bacteria were harvested at 3,500×g for 20 minutes and washed three times with PBS contain-ing 0.1% BSA. The resulting crude cell lysate pellets were stored at −80° C. The other pellets, collected from ˜960 mL of culture, were resuspended in 120 mL of cold, 25 mM citrate buffer (pH 3.2) and incubated with gentle shaking for 2 h. Every 20-30 mins, each sample vigorously vortexed for ˜30 s. Both OMVs and PCs were collected by centrifugation at 21,000×g for 20 mins, resuspended in 25 mM citrate buffer, each sample split into 3 equal volumes, and applied to a discontinuous sucrose gradient (56%; 42%; 25%). All 6 tubes were centrifuged at 102,500×g for ˜18 hours at 4° C. OMV and PC fractions, from each sample, were remove by needle aspiration, pooled and collected by centrifugation at 142,000×g for 6 hours. Each fraction, from each sample, were resuspended in 25 mM citrate buffer and applied to continuous sucrose (10-42%) and separated as described above. Both PC and OMV were collected, once again, by needle aspiration, diluted in PBS, and collected by centrifugation at 12,500×g for 20 mins, and 142,000×g for 6 hours, respectively. The resulting material was resuspended in PBS, aliquoted, and stored at −80° C.

### Immunoblots

All antibodies used in this study have been previously characterized. Anti-FlaB [88] loading control, and anti-NapA [35] were graciously provided by Melissa Caimano and Frank Ghera-dini, respectively. Rabbit anti-serum raised against rOspA was purchased from Rockland Inc. Polyclonal anti-PG rabbit serum was recently validated and provided by the Christine Jacobs-Wagner lab. Dilutions for western blots were as follows: Anti-FlaB (1:1000); anti-NapA (1:8000); anti-OspA (1:1000); anti-PG (1:90). A 1:8000 dilution of rabbit IgG:HRP (Jackson labs) was used to detect all primary antibodies, with the exception of FlaB, which was detected with rat IgG:HRP (Jackson labs), used at the same dilution. All secondary antibodies were detected by chemiluminescence using SuperSignal West Pico PLUS (Thermo Scientific) detec-tion reagents and imaged with a Syngene G:box (Imgene Technologies).

### Immunofluorescence

5A11 and napA/5A11 were cultured (40 mL) to a final density of 5×107 cells/mL. Cells were fixed by quickly adding a freshly opened ampule of paraformaldehyde to final concentration of 2% (vol/vol). Cells were fixed, with gentle agitation for 10 minutes at room temperature, and the reaction terminated on ice for 30 minutes. After harvesting and washing the fixed cells, they were stored at −20° C.

Fixed cells were spotted on poly-L-lysine-coated slides. After washing with PBS supplemented with 0.05% Tween 20 (PBS-T) to remove unbound material, Applicant proceeded with different methods to permeabilize the cells. No permeabilization consisted of three PBS washes, and three Seablock (Abcam) prior to block the cells with Seablock for 2 hours. Inner membrane permeabilization involved treating cells for 10 minutes, at room temperature, with 50% methanol. Methanol was removed by aspiration, cells washed three times with PBS, then washed three times with Seablock prior to blocking for 2 hours with Seablock. For Inner/outer mem-brane permeabilization cells were first treated with 0.03% SDS for 3 minutes at 37° C. SDS was removed with PBS washes, and fixed samples were subsequently treated with 1 mg/mL of lysozyme (Sigma-Aldrich). Permeabilized samples washed three times with PBS, then washed three times with Seablock (Abcam) prior to blocking for 2 hours with Seablock. Immunolabel-ing each target were the same for each sample and treatment. All antibodies were diluted in Seablock. Primary: secondary antibody pairs and dilutions were as follows: 1) GFP: mouse α-GFP, [1:100] (Sigma Aldrich); Goat α-mouse IgG:Alexa Fluor 647 [1:250] (Jackson Laborato-ries). 2) FlaB: rat α-FlaB, [1:75]; Goat α-rat IgG:Alexa Fluor 555 [1:250] (ThermoFisher). 3) NapA: rabbit α-NapA [1:400]; Donkey α-Rabbit IgG:Alexa Fluor 647 [1:250] (Jackson Labora-tories). Primary incubations occurred at room temperature for 1-hour, unbound material was washed 15 times with PBS-T, and probed with secondary antibodies for 1 hour. Unbound sec-ondary antibodies were washed 15 times. Slides were treated with SlowFade (ThermoFisher) and imaged as described below.

The immunofluorescence procedure for purified PG and PG-linked NapA were identical to that previously described [33]. Briefly, pre- and post-trypsin treated PG preparations were spotted onto poly-L-lysine-coated slides. After washing with PBS supplemented with 0.05% Tween 20 (PBS-T) to remove unbound material, samples were blocked with Seablock (Abcam) for 2 hours. Anti-NapA (1:400) was co-incubated with 5 μg/mL WGA:Alexa 350 on each sam-ple for 1 hour and washed 15 times with PBS-T. Anti-NapA was detected with the Goat α-rab-bit IgG:Cy3 conjugated antibody (Jackson laboratories), diluted 1:250. Control reactions included secondary antibody, without primary. Samples were treated with SlowFade and imaged as described below.

### PBMC Stimulations

Pooled cryo-preserved PBMCs were seeded in 12-well plates at 2×106 cells/well in Lympho-cyte culture media (Zen-Bio), pre-equilibrated to 37° C. under 5% CO2. Cells rested for 18 h under these conditions prior to stimulation. Following stimulation for ˜72 hours with 25 μg/mL of B. burgdorferi PG, the cells were harvested by centrifugation at 800×g for 5 min at 15° C. The supernatants were collected, aliquoted, and kept at −80° C. prior to cytokine analysis. Cytokine analyses were performed on stimulated PBMC supernatants, diluted 1:4, according to the manufacturers (Abcam ELISA Kit IL-17A/F). Results were normalized to controls stim-ulation with diluent (PBS control). Statistical significance was determined by an unpaired Stu-dent's T Test. Unless stated in the text, statistical significance was set at p<0.05.

### Microscopy and Image Acquisition

Samples were immobilized by poly-L-lysine-coated slides. Epifluorescence microscopy was performed on a Zeiss Axio Observer equipped with a Hamamatsu Orca-Flash 4.0 V3 Digital CMOS camera, Colibri 7, and an oil-immersion phase-contrast objective Plan Apochromat 100×/1.45 N.A. (Nikon). Phase contrast and epifluorescence exposures were 100 ms and 500 ms, respectively.

### Microscopy Analysis

Data and statistical tests were performed using Graph Pad Prism 6.0 Software Inc. Automated sacculi detection was achieved using Oufti [89] on inverted WGA signal. Sacculi were detected using a subpixel logarithmic algorithm that was optimized for Applicant's images. The localization of NapA was identified by a Gaussian fit to the NapA signal for each cell mesh in the population (spotDetection via Oufti). The NapA locations were then normalized and plotted relative to NapA signal intensity. NapA locations were then binned; shaded region represents the stan-dard deviation for each bin. Data attained from cell meshes were graphed using MatLab 2019a. The codes used to generate FIG. 2E are provided in 51 Text “MatlabScripts” of Davis et al., 2021. PloS Pathog 17(5): e1009546, which is incorporated by reference as if expressed in its entirety herein.

### Cryo-Electron Tomography

Frozen-hydrated specimens were prepared as previously described [35]. Briefly, B. burgdorferi culture was mixed with 10 nm colloidal gold and was then deposited onto freshly glow-dis-charged, holey carbon EM grids for 1 min. Grids were blotted with filter paper and then rap-idly frozen in liquid ethane, using a homemade gravity-driven plunger apparatus. Frozen-hydrated specimens were imaged at −170° C. using a Titan Krios electron microscope (Thermo Fisher) equipped with a field emission gun and a K2 Summit direct detector device (Gatan). The microscope was operated at 300 kV with a magnification of 53,000×, resulting in an effective pixel size of 2.7 Å at the specimen level. SerialEM [90] was used to collect tilt series with a cumulative dose of ˜60 e-/Å2. IMOD [91] was used for alignment and reconstruction.

### Stress Tests and Colony Forming Units

Initial studies geared towards understanding both permissive and restrictive growth conditions upon stress were performed in microplates with serial dilutions. Each strain was cultured to ˜1×10⁶ cells/mL and subsequently back diluted to a final concentration of 1×104 cells/mL in fresh B SKIT containing cell-wall stress. Lysozyme and NaCl were serially diluted 1:1. The final concentration of Lysozyme ranged from 2 mg/mL to 0 mg/mL, while NaCl varied from 500 nM to 7.8 mM. Applicant also reports the osmolality of each culture media condition that was spiked with NaCl. Osmolality was determined using Fiske Micro-Osmometer Model 210, following manufacturers recommended procedures. Both microtiter plates contained 4 wells of uninoculated media, which served as a negative control. These 96 well plates were allowed to incubate at 37° C. under 5% CO₂ for 6 days. Afterwards, they were placed at ambient conditions for 2 hours before being imaged.

### Colony Forming Units

Parental and NapA mutant bacteria were cultured to ˜6.5×10⁶ cells/mL and back diluted to a final starting concentration of 106 cells/mL. Each culture was stressed with Lysozyme (0.37 mg/mL) or NaCl (0.111 M) for 24 hours without additional antibiotic selection. Afterwards, each culture was plated using standard methods [92] and cultured at 37° C. under 5% CO₂. CFUs were determined for strain 5A11 after 3 weeks; napA mutant CFUs were counted after 6 weeks.

### Neutrophil Migration

The microfluidic competitive chemotaxis-chip (μC3) [55] was used to perform each migration assay. This device allowed for the creation of a dual gradient through two opposing chemoattractant reservoirs. The central cell-loading chamber is connected to the two reservoirs by perpendicular cell migration ladders (measured 10 μm wide×10 μm tall). Device fabrication was as previously [55]. Briefly, two layers of photoresist (SUB, MicroChem), the first one 10 μm thin (corresponding to the migration channels) and the second one 70 μm thick (corresponding to the neutrophil loading chamber) were patterned on one silicon wafer sequentially using two photolithographic masks and processing cycles according to the instructions from the manufacturer. The wafer with patterned photoresist was used as a mold to produce polydimethylsiloxane (PDMS) (Sylgard 184, Elsworth Adhesives, Wilmington, Mass.) devices, which were then bonded to the base of glass-bottom 6-well plates (MatTek Corp., Ashland, Mass.), using an oxygen plasma machine (Nordson March, Concord, Calif.). Prior to each migration assay, the device was primed with fibronectin (Sigma-Aldrich, St. Louis, Mo.) (11 μg/mL). After priming with fibronectin, each device was covered in 4 mL complete media. Samples were loaded into one chemoattractant reservoir of each corresponding device using a trimmed gel loading pipette tip. Formylmethionine-leucyl-phenylalanine (fMLP, Sigma-Aldrich, St. Louis, Mo.) (10 nM) and Leukotriene B4 (LTB4, Cayman Chemical, Ann Arbor, Mich.) (100 nM) served as positive controls in the chemotaxis assay and were loaded in the same manner. The second chemoattractant reservoir was filled with complete media to measure chemorepulsion from the sample. Complete media in both reservoirs served as a negative control in the chemotaxis assay. dHL-60 cells were loaded into the central cell-loading chamber in the ladder device with a gel loading pipette tip. The media was removed and replaced with new complete media after the dHL-60 cells were loaded.

### Chemotaxis Imaging and Measurements

Each assay was visualized on a fully automated Nikon TiE microscope using a Plan Fluor 10×Ph1 DLL (NA=0.3) lens with a biochamber heated to 37° C. with 5% CO₂. Image capture was performed using NIS-elements (Nikon Inc., Melville, N.Y.). Experiments were run under the microscope for 5 hours with brightfield and fluorescent images taken at 2-minute intervals. Image analysis of cell migration counts was analyzed automatically using ImageJ Cell tracking was conducted using an automated tracker, TrackMate [93] (custom tracking and analysis codes are available for download at https://github.com/boribong/Single-Cell-Migration-Tracking) and ImageJ software (NIH). Cell migration parameters have been defined previously [55].

Statistical Analysis of dHL-60 Cell Chemotaxis Towards NapA

All experiments were performed and replicated at least three times, unless otherwise stated. Statistical analysis was performed using Prism software (GraphPad Software, La Jolla, Calif.). Data expressed as means±standard deviations. To compare the migration between the different 5A11 and 5A11/napA samples, Applicant used a one-way ANOVA and Turkey's Multiple Com-parison test. To compare the migration toward or away within the different 5A11 and 5A11/napA samples, Applicant used a Student's t-test. Differences were considered statistically significant for p<0.05.

hNOD2 Activation Assay

To quantify the amount of Muramyl-dipeptide (MDP) present in OMVs and PCs Applicant used the hNOD2 reporter assay (Invivogen) as described previously [16]. Control reactions included 20 ug/mL of the RIP2 inhibitor gefitinib (Sigma Aldrich). MDP (50 ng/mL, Invivogen) served as the positive control.

### Supplemental Information

Table 1 shows a summary of LC-MS results from PAP preliminary screen. LC-MS results from three biological replicates of PG-associated protein analysis following trypsin cleavage. Data reported represent the mean of all three experiments+/−the standard deviation (SD) for the following categories: MASCOT score; number of unique peptides identified per experiment (# peptides); number of peptide-spectrum matches per experiment (# PSM). Note: Only reliable hits that were observed in two or more experiments were reported.

TABLE 1

| Accession | Name | MW | Observed | | | |
|---|---|---|---|---|---|---|
| (kDa) | mean(+/−SD) | mean(+/−SD) | mean(+/−SD) | MASCOT | # peptides | # PSM |
| BB0690 | NapA | 21.3 | 3/3 | 244 (175) | 3.7 (2) | 23 (20) |
| BB0744 | P83/P100 antigen | 79.9 | 2/3 | 399 (656) | 3.7 (5.5) | 20 (33) |
| BB0476 | Elongation Factor Tu | 43.6 | 2/3 | 227 (272) | 5 (5) | 15 (18) |
| BB0388 | RpoC | 155 | 2/3 | 118 (142) | 5 (1.4) | 12 (1.4 |

Table 2 shows a summary of LC-MS results from stringent PAP screen. LC-MS results from two bio-logical replicates of PG-associated protein analysis following trypsin cleavage. Data reported represent the mean of both experiments+/−the standard deviation (SD) for the following categories: MASCOT score; number of unique peptides identified per experiment (# peptides); number of peptide-spectrum matches per experiment (# PSM). Note that Table 2 differs from Table 1 in the sample preparation. Whereas data presented in Table 1 were from a single SDS solubilization step, Table 2 represents results from a second SDS solubilization step.

TABLE 2

| | | MW | | MASCOT | # peptides | # PSM |
|---|---|---|---|---|---|---|
| Accession | Name | (kDa) | Observed | mean(+/−SD) | mean(+/−SD) | nean(+/−SD) |
| BB0690 | NapA | 21.3 | 2/2 | 181 (34) | 3.5 (2) | 12 (3) |
| BB0744 | P83/P100 antigen | 79.9 | 2/2 | 54 (61) | 1.5 (0.7) | 3 (1.4) |

Table 3 shows a parental clone 5A11 mutations relative to B31 reference genome. All mutations that differ from the B31 type strain are shown with the exception of the hypervariable vlsE expression locus. (S) substitution; (A) addition; (D) deletion.

TABLE 3

| Mutation | Event | Coordinate | Location | Result |
|---|---|---|---|---|
| S | G→T | 10,883 | Coding-OspB | G199V |
| S | C→A | 28,269 | Coding-BBN41 | Q82K |
| S | C→T | 17,924 | Intergenic | — |
| A | +C | 3,140 | Intergenic | — |
| S | G→A | 56,157 | Coding-BB0059 | V42I |
| A | +A | 138,870 | Intergenic | — |
| A | +T | 366,152 | Intergenic | — |
| A | +A | 422,314 | Intergenic | — |
| D | −G | 515,969 | Intergenic | — |
| D | −C | 516,002 | Intergenic | — |
| D | −G | 516,098 | Intergenic | — |
| S | T→A | 528,031 | Intergenic | — |
| A | +G | 532,509 | Intergenic | — |
| A | +T | 540,032 | Intergenic | — |
| S | T→G | 747,897 | Intergenic | — |
| A | +A | 862,670 | Intergenic | — |

Table 4 shows 5A11/napA mutations relative to B31 reference genome. All mutations that differ from the B31 type strain are shown with the exception of the hypervariable vlsE expression locus. (S) substitution; (A) addition; (D) deletion.

TABLE 4

| Mutation | Event | Coordinate | Location | Result |
|---|---|---|---|---|
| S | G→T | 10,883 | Coding-OspB | G199V |
| S | C→A | 28,269 | Coding-BBN41 | Q82K |
| S | C→T | 17,924 | Intergenic | — |
| A | +C | 3,140 | Intergenic | — |
| S | G→A | 56,157 | Coding-BB0059 | V42I |
| A | +A | 138,870 | Intergenic | — |
| A | +T | 366,152 | Intergenic | — |
| A | +A | 422,314 | Intergenic | — |
| D | −A | 424,631 | Intergenic | — |
| D | −G | 515,969 | Intergenic | — |
| D | −C | 516,002 | Intergenic | — |
| D | −G | 516,098 | Intergenic | — |
| S | T→A | 528,031 | Intergenic | — |
| A | +G | 532,509 | Intergenic | |
| A | +T | 540,032 | Intergenic | |
| D | Allele exchange | 731,203-731,758 | Coding-NapA | napA |
| S | T→G | 747,897 | Intergenic | |
| A | +A | 862,670 | Intergenic | |

TABLE 5
shows the definition of migratory parameters.

| Migratory Phenotype | Definition | Units |
|---|---|---|
| Percentage of Cells | Number of Cells | Percentage (%) |
| Migrated | Migrated/Average of Cells in | |
| | Central Loading Chamber × | |
| | 100 | |
| dHL-60 Cell | Distance Cell Traveled/Time | μm/min |
| Velocity | Elapsed | |
| Non-Directional | dHL-60 cells that enter the | Number of Cells |
| Migration | cell mazes | |
| Oscillatory | Cells that change direction in | Number of Cells |
| Migration | the x or y plane ≥ 3 times | |

FIG. 7A shows results from a lysozyme and NaCl stress test. Both 5A11 and 5A11/napA strains were grown to 1×10⁴ cells/mL in BSK II at 37 degrees C. media prior to adding increasing amounts of Lysozyme (0 to 2 mg/mL) (left) or NaCl (7.8 to 500 mM) (right). Final osmolality of culture media is also shown (380 to 1410 mOsm). Cells were allowed to grow for one week in a 96 well plate prior to growth analysis using spectrophotometry. FIG. 7B shows growth curves. 5A11 and 5A11/napA were grown at a starting concentration of 1×10³ cells/mL in BSK II media. Cells were enumerated roughly every 24 hours for 10 days with the exception of the first count which occurred 48 hours after inoculation. Note that for data presented in FIG. 3A-3D the concentrations of Lysozyme and NaCl tested were in between wells 5-6 and 4-5, respectively.

FIG. 8A-8B shows (FIG. 8A) SDS PAGE and immunoblot analysis of Outer Membrane Vesicle and Protoplasmic Cylinder preparations. Both 5A11 and 5A11/napA strains were cultured to late-log, cell were harvested, and fractionated into outer membrane vesicles (OMV) and protoplasmic cylinders (PC). Each preparation was separate by SDS PAGE and visualized by Sypro Ruby stain. Asterisk (*) indicate bands only present in OMVs; and (FIG. 8B) NapA Immunoblot of samples prepared as described above.

FIG. 9A-9B shows. dHL60 cells migrated toward NapA-associated PG shows less non-dysfunctional migratory patterns in comparison to other preparations. FIG. 9A shows cells migrating toward NapAassociated PG shows lowest number of cells displaying of non-directional migration (n=23). FIG. 9B shows Cells migrating toward NapA-associated PG shows lowest number of cells showing oscillatory migration (n=13).

FIG. 10A-10B shows Fig. dHL-60 cells migrating toward Nap-A associated PG show higher velocity in com-parison to other preparations. Single cell velocity values are plotted over a box plot showing range of values. FIG. 10A shows Cells migrating toward PG bait samples and chemoattractants show Nap-A associated PG has a similar velocity (10.94±4.79 μm/min) to known chemoattractants LTB4 (7.04±4.90 μm/min) and fMLP (6.93±4.40 μm/min). FIG. 10B shows cells migrating away from PG bait samples and chemoattractants show similar velocities. To evaluate differences between responses ANOVA were performed with Turkey's correction for multiple comparisons (*=p<0.05, ***=p<0.001).

FIG. 11A-11C shows a phylogenetic analysis of Dps/NapA. FIG. 11A shows a phylogenic analysis of Dps/NapA homologues in Borreliae, Helicobacter pylori, Treponema pallidum, Leptospira interrogans, Yersinia pestis, and Escherichia coli FIG. 11B shows an amino acid alignment of Dps/NapA homologues from bacteria in FIG. 11A. The Lysine-rich DNA binding domain is underlined (blue) FIG. 11C shows a zoomed in amino acid sequence of the C-terminus of Dps/NapA homologues.

### References for Example 1

1. Prevention CfDCa. Recent Lyme disease surveillance data Nov. 7, 2019 [Available from: https://www.cdc.gov/lyme/datasurveillance/recent-surveillance-data.html.
2. Kugeler K J, Schwartz A M, Delorey M J, Mead P S, Hinckley A F. Estimating the Frequency of Lyme Dis-ease Diagnoses, United States, 2010-2018. Emerg Infect Dis. 2021; 27(2):616-9. https://doi.org/10. 3201/eid2702.202731 PMID: 33496229
3. Mead P S. Epidemiology of Lyme disease. Infectious Disease Clinics. 2015; 29(2):187-210. https://doi. org/10.1016/j.idc.2015.02.010 PMID: 25999219
4. Eisen R J, Eisen L, Beard C B. County-scale distribution of Ixodes scapularis and Ixodes pacificus (Acari: Ixodidae) in the continental United States. J Med Entomol. 2016; 53(2):349-86. https://doi.org/10.1093/jme/tjv237 PMID: 26783367
5. Bisanzio D, Fernández M P, Martello E, Reithinger R, Diuk-Wasser M A. Current and future spatiotempo-ral patterns of Lyme disease reporting in the northeastern United States. JAMA Netw Open. 2020; 3(3): e200319. https://doi.org/10.1001/jamanetworkopen.2020.0319 PMID: 32125426
6. Stanek G, Strle F. Lyme borreliosis-from tick bite to diagnosis and treatment. FEMS Microbiol Rev. 2018; 42(3):233-58. https://doi.org/10.1093/femsre/fux047 PMID: 29893904
7. Hatchette T F, Davis I, Johnston B L. Lyme disease: clinical diagnosis and treatment. Can Commun Dis Rep. 2014; 40(11):194-208. https://doi.org/10.14745/ccdr.v40i11a01 PMID: 29769842
8. Wormser G P, Dattwyler R J, Shapiro E D, Halperin J J, Steere A C, Klempner M S, et al. The clinical assessment, treatment, and prevention of lyme disease, human granulocytic anaplasmosis, and babe-siosis: clinical practice guidelines by the Infectious Diseases Society of America. Clin Infect Dis. 2006; 43(9):1089-134. https://doi.org/10.1086/508667 PMID: 17029130
9. Steere A C, Strle F, Wormser G P, Hu L T, Branda J A, Hovius J W, et al. Lyme borreliosis. Nat Rev Dis Primers. 2016; 2:16090. https://doi.org/10.1038/nrdp.2016.90 PMID: 27976670
10. Steere A C. Posttreatment Lyme disease syndromes: distinct pathogenesis caused by maladaptive host responses. J Clin Invest. 2020; 130(5):2148-51. https://doi.org/10.1172/JCI138062 PMID: 32281948
11. Sulka K B, Strle K, Crowley J T, Lochhead R B, Anthony R, Steere A C. Correlation of Lyme disease-associated IgG4 autoantibodies with synovial pathology in antibiotic-refractory Lyme arthritis. Arthritis Rheumatol. 2018; 70(11):1835-46. https://doi.org/10.1002/art.40566 PMID: 29790305
12. Drouin E E, Seward R J, Strle K, McHugh G, Katchar K, Londoño D, et al. A novel human autoantigen, endothelial cell growth factor, is a target of T and B cell responses in patients with Lyme disease. Arthri-tis Rheum. 2013; 65(1):186-96. https://doi.org/10.1002/art.37732 PMID: 23044924
13. Crowley J T, Strle K, Drouin E E, Pianta A, Arvikar S L, Wang Q, et al. Matrix metalloproteinase-10 is a target of T and B cell responses that correlate with synovial pathology in patients with antibiotic-refrac-tory Lyme arthritis. J Autoimmun. 2016; 69:24-37. https://doi.org/10.1016/j.jaut.2016.02.005 PMID: 26922382
14. Pianta A, Drouin E E, Crowley J T, Arvikar S, Strle K, Costello C E, et al. Annexin A2 is a target of autoim-mune T and B cell responses associated with synovial fibroblast proliferation in patients with antibiotic-refractory Lyme arthritis. Clin Immunol. 2015; 160(2):336-41. https://doi.org/10.1016/j.clim.2015.07.005 PMID: 26187145
15. Strle K, Shin J J, Glickstein L J, Steere A C. Association of a Toll-like receptor 1 polymorphism with heightened Thl inflammatory responses and antibiotic-refractory Lyme arthritis. Arthritis Rheum. 2012; 64(5):1497-507. https://doi.org/10.1002/art.34383 PMID: 22246581
16. Jutras B L, Lochhead R B, Kloos Z A, Biboy J, Strle K, Booth C J, et al. Borrelia burgdorferi peptidoglycan is a persistent antigen in patients with Lyme arthritis. Proc Natl Acad Sci USA. 2019; 116(27):13498-507. https://doi.org/10.1073/pnas.1904170116 PMID: 31209025
17. Costerton J W, Ingram J M, Cheng K J. Structure and function of the cell envelope of gram-negative bac-teria. Bacteriol Rev. 1974; 38(1):87-110. PMID: 4601163
18. Gan L, Chen S, Jensen G J. Molecular organization of Gram-negative peptidoglycan. Proc Natl Acad Sci USA. 2008; 105(48):18953-7. https://doi.org/10.1073/pnas.0808035105 PMID: 19033194
19. Parry B R, Surovtsev I V, Cabeen M T, O'Hern C S, Dufresne E R, Jacobs-Wagner C. The bacterial cyto-plasm has glass-like properties and is fluidized by metabolic activity. Cell. 2014; 156(1-2):183-94. https://doi.org/10.1016/j.cell.2013.11.028 PMID: 24361104
20. Kovacs-Simon A, Titball R W, Michell S L. Lipoproteins of bacterial pathogens. Infect Immun. 2011; 79(2):548-61. https://doi.org/10.1128/IAI.00682-10 PMID: 20974828
21. Cascales E, Bernadac A, Gavioli M, Lazzaroni J C, Lloubes R. Pal lipoprotein of Escherichia coli plays a major role in outer membrane integrity. J Bacteriol. 2002; 184(3):754-9. https://doi.org/10.1128/jb.184. 3.754-759.2002 PMID: 11790745
22. Haake D A. Spirochaetal lipoproteins and pathogenesis. Microbiol-Uk. 2000; 146:1491-504. https://doi.org/10.1099/00221287-146-7-1491 PMID: 10878114
23. Henderson B, Martin A. Bacterial virulence in the moonlight: multitasking bacterial moonlighting proteins are virulence determinants in infectious disease. Infect Immun. 2011; 79(9):3476-91. https://doi.org/10. 1128/IAI.00179-11 PMID: 21646455
24. Fraser C M, Casjens S, Huang W M, Sutton G G, Clayton R, Lathigra R, et al. Genomic sequence of a Lyme disease spirochaete, Borrelia burgdorferi. Nature. 1997; 390(6660):580-6. https://doi.org/10. 1038/37551 PMID: 9403685
25. Casjens S, Palmer N, van Vugt R, Huang W M, Stevenson B, Rosa P, et al. A bacterial genome in flux: the twelve linear and nine circular extrachromosomal DNAs in an infectious isolate of the Lyme disease spirochete Borrelia burgdorferi. Mol Microbiol. 2000; 35(3):490-516. https://doi.org/10.1046/j.1365-2958.2000.01698.x PMID: 10672174
26. Radolf J D, Caimano M J, Stevenson B, Hu L T. Of ticks, mice and men: understanding the dual-host life-style of Lyme disease spirochaetes. Nat Rev Microbiol. 2012; 10(2):87-99. https://doi.org/10.1038/nrmicro2714 PMID: 22230951
27. Pappas C J, Iyer R, Petzke M M, Caimano M J, Radolf J D, Schwartz I. Borrelia burgdorferi requires glyc-erol for maximum fitness during the tick phase of the enzootic cycle. PLoS Pathog. 2011; 7(7): e1002102. https://doi.org/10.1371/journal.ppat.1002102 PMID: 21750672
28. Crowley J T, Toledo A M, LaRocca T J, Coleman J L, London E, Benach J L. Lipid exchange between Bor-relia burgdorferi and host cells. PLoS Pathog. 2013; 9(1):e1003109. https://doi.org/10.1371/journal. ppat.1003109 PMID: 23326230
29. Zuckert W R. Secretion of bacterial lipoproteins: through the cytoplasmic membrane, the periplasm and beyond. Biochim Biophys Acta. 2014; 1843(8):1509-16. https://doi.org/10.1016/j.bbamcr.2014.04.022 PMID: 24780125
30. Wolgemuth C W, Charon N W, Goldstein S F, Goldstein R E. The flagellar cytoskeleton of the spiro-chetes. J Mol Microbiol Biotechnol. 2006; 11(3-5):221-7. https://doi.org/10.1159/000094056 PMID: 16983197
31. Motaleb M A, Comm L, Bono J L, Elias A F, Rosa P, Samuels D S, et al. Borrelia burgdorferi periplasmic flagella have both skeletal and motility functions. Proc Natl Acad Sci USA. 2000; 97(20):10899-904. https://doi.org/10.1073/pnas.200221797 PMID: 10995478
32. Beck G, Benach J L, Habicht G S. Isolation, preliminary chemical characterization, and biological activity of Borrelia burgdorferi peptidoglycan. Biochem Biophys Res Commun. 1990; 167(1):89-95. https://doi.org/10.1016/0006-291x(90)91734-a PMID: 2310405
33. Jutras B L, Scott M, Parry B, Biboy J, Gray J, Vollmer W, et al. Lyme disease and relapsing fever Borrelia elongate through zones of peptidoglycan synthesis that mark division sites of daughter cells. Proc Natl Acad Sci USA. 2016; 113(33):9162-70. https://doi.org/10.1073/pnas.1610805113 PMID: 27506799
34. Li X, Pal U, Ramamoorthi N, Liu X, Desrosiers D C, Eggers C H, et al. The Lyme disease agent Borrelia burgdorferi requires BB0690, a Dps homologue, to persist within ticks. Mol Microbiol. 2007; 63(3):694-710. https://doi.org/10.1111/j.1365-2958.2006.05550.x PMID: 17181780
35. Seshu J, Boylan J A, Gherardini F C, Skare J T. Dissolved oxygen levels alter gene expression and anti-gen profiles in Borrelia burgdorferi. Infect Immun. 2004; 72(3):1580-6. https://doi.org/10.1128/iai.72.3. 1580-1586.2004 PMID: 14977964
36. Calhoun L N, Kwon Y M. Structure, function and regulation of the DNA-binding protein Dps and its role in acid and oxidative stress resistance in Escherichia coli: a review. J Appl Microbiol. 2011; 110(2):375-86. https://doi.org/10.1111/j.1365-2672.2010.04890.x PMID: 21143355
37. Haikarainen T, Papageorgiou A C. Dps-like proteins: structural and functional insights into a versatile protein family. Cell Mol Life Sci. 2010; 67(3):341-51. https://doi.org/10.1007/s00018-009-0168-2 PMID: 19826764
38. Cooksley C, Jenks P J, Green A, Cockayne A, Logan R P, Hardie K R. NapA protects Helicobacter pylori from oxidative stress damage, and its production is influenced by the ferric uptake regulator. J Med Microbiol. 2003; 52(Pt 6):461-9. https://doi.org/10.1099/jmm.0.05070-0 PMID: 12748264
39. Wang P, Lutton A, Olesik J, Vali H, Li X. A novel iron- and copper-binding protein in the Lyme disease spirochaete. Mol Microbiol. 2012; 86(6):1441-51. https://doi.org/10.1111/mmi.12068 PMID: 23061404
40. Codolo G, Amedei A, Steere A C, Papinutto E, Cappon A, Polenghi A, et al. Borrelia burgdorferi NapA-driven Th17 cell inflammation in lyme arthritis. Arthritis Rheum. 2008; 58(11):3609-17. https://doi.org/10.1002/art.23972 PMID: 18975343
41. Codolo G, Papinutto E, Polenghi A, D'Elios M M, Zanotti G, de Bernard M. Structure and immunomodu-latory property relationship in NapA of Borrelia burgdorferi. Biochim Biophys Acta. 2010; 1804(12):2191-7. https://doi.org/10.1016/j.bbapap.2010.09.004 PMID: 20851780
42. Codolo G, Bossi F, Durigutto P, Bella C D, Fischetti F, Amedei A, et al. Orchestration of inflammation and adaptive immunity in Borrelia burgdorferi-induced arthritis by neutrophil-activating protein A. Arthri-tis Rheum. 2013; 65(5):1232-42. https://doi.org/10.1002/art.37875 PMID: 23371320
43. Troxell B, Ye M, Yang Y, Carrasco S E, Lou Y, Yang X F. Manganese and zinc regulate virulence deter-minants in Borrelia burgdorferi. Infect Immun. 2013; 81(8):2743-52. https://doi.org/10.1128/IAI.00507-13 PMID: 23690398
44. Takacs N C, Kloos A Z, Scott M, Rosa A P, Jacobs-Wagner C. Fluorescent proteins, promoters, and selectable markers for applications in the Lyme disease spirochete Borrelia burgdorferi. 2018; 84(24): e01824-18.
45. Chang Y, Moon K H, Zhao X, Norris S J, Motaleb M A, Liu J. Structural insights into flagellar stator-rotor interactions. Elife. 2019; 8. https://doi.org/10.7554/eLife.48979 PMID: 31313986
46. Mulay V, Caimano M J, Liveris D, Desrosiers D C, Radolf J D, Schwartz I. Borrelia burgdorferi BBA74, a periplasmic protein associated with the outer membrane, lacks porin-like properties. J Bacteriol. 2007; 189(5):2063-8. https://doi.org/10.1128/J B.01239-06 PMID: 17189354
47. Koetsveld J, Draga ROP, Wagemakers A, Manger A, Oei A, Visser C E, et al. In Vitro Susceptibility of the Relapsing-Fever Spirochete Borrelia miyamotoi to Antimicrobial Agents. Antimicrob Agents Che-mother. 2017; 61(9). https://doi.org/10.1128/AAC.00535-17 PMID: 28674060
48. Dever L L, Jorgensen J H, Barbour A G. In vitro antimicrobial susceptibility testing of Borrelia burgdorferi: a microdilution MIC method and time-kill studies. J Clin Microbiol. 1992; 30(10):2692-7. https://doi.org/10.1128/JCM.30.10.2692-2697.1992 PMID: 1400969
49. Skare J T, Shang E S, Foley D M, Blanco D R, Champion C I, Mirzabekov T, et al. Virulent strain associ-ated outer membrane proteins of Borrelia burgdorferi. J Clin Invest. 1995; 96(5):2380-92. https://doi.org/10.1172/JCI118295 PMID: 7593626
50. Yang X, Promnares K, Qin J, He M, Shroder D Y, Kariu T, et al. Characterization of multiprotein com-plexes of the Borrelia burgdorferi outer membrane vesicles. J Proteome Res. 2011; 10(10):4556-66. https://doi.org/10.1021/pr200395b PMID: 21875077
51. Tigno-Aranjuez J T, Asara J M, Abbott D W. Inhibition of RIP2's tyrosine kinase activity limits NOD2-driven cytokine responses. Genes Dev. 2010; 24(23):2666-77. https://doi.org/10.1101/gad.1964410 PMID: 21123652
52. Schwechheimer C, Kuehn M J. Outer-membrane vesicles from Gram-negative bacteria: biogenesis and functions. Nat Rev Microbiol. 2015; 13(10):605-19. https://doi.org/10.1038/nrmicro3525 PMID: 26373371
53. Oosting M, ter Hofstede H, van de Veerdonk F L, Sturm P, Kullberg B J, van der Meer J W, et al. Role of interleukin-23 (IL-23) receptor signaling for IL-17 responses in human Lyme disease. Infect Immun. 2011; 79(11):4681-7. https://doi.org/10.1128/IAI.05242-11 PMID: 21896776
54. Lochhead R B, Ordoñez D, Arvikar S L, Aversa J M, Oh L S, Heyworth B, et al. Interferon-gamma produc-tion in Lyme arthritis synovial tissue promotes differentiation of fibroblast-like synoviocytes into immune effector cells. Cell Microbiol. 2019; 21(2):e12992. https://doi.org/10.1111/cmi.12992 PMID: 30550623
55. Boribong B P, Lenzi M J, Li L, Jones C N. Super-low dose lipopolysaccharide dysregulates neutrophil migratory decision-making. Front Immunol. 2019; 10:359. https://doi.org/10.3389/fimmu.2019.00359 PMID: 30915068
56. Schulze R J, Zuckert W R. Borrelia burgdorferi lipoproteins are secreted to the outer surface by default. Mol Microbiol. 2006; 59(5):1473-84. https://doi.org/10.1111/j 0.1365-2958.2006.05039.x PMID: 16468989
57. Zückert W R. Protein Secretion in Spirochetes. Microbiol Spectr. 2019; 7(3). https://doi.org/10.1128/microbiolspec.PSIB-0026-2019 PMID: 31198130
58. Ratet G, Santecchia I, Fanton d'Andon M, Vernel-Pauillac F, Wheeler R, Lenormand P, et al. LipL21 lipoprotein binding to peptidoglycan enables Leptospira interrogans to escape NOD1 and NOD2 recog-nition. PLoS Pathog. 2017; 13(12):e1006725. https://doi.org/10.1371/journal.ppat.1006725 PMID:
59. Chou S, Daugherty M D, Peterson S B, Biboy J, Yang Y, Jutras B L, et al. Transferred interbacterial antagonism genes augment eukaryotic innate immune function. Nature. 2015; 518(7537):98-101. https://doi.org/10.1038/nature13965 PMID: 25470067
60. Oliva Chavez A S, Shaw D K, Munderloh U G, Pedra J H. Tick humoral responses: Marching to the beat of a different drummer. Front Microbiol. 2017; 8:223. https://doi.org/10.3389/fmicb.2017.00223 PMID: 28261180
61. Kim T K, Tirloni L, Pinto A F, Moresco J, Yates J R 3rd, da Silva Vaz I Jr, et al. Ixodes scapularis tick saliva proteins sequentially secreted every 24 h during blood feeding. PLoS Negl Trop Dis. 2016; 10(1): e0004323. https://doi.org/10.1371/journal.pntd.0004323 PMID: 26751078
62. Dramsi S, Magnet S, Davison S, Arthur M. Covalent attachment of proteins to peptidoglycan. FEMS Microbiol Rev. 2008; 32(2):307-20. https://doi.org/10.1111/j 0.1574-6976.2008.00102.x PMID: 18266854
63. Cafiero T, Toledo A. Borrelia burgdorferi surface exposed GroEL is a multifunctional protein. Patho-gens. 2021; 10(2):226. https://doi.org/10.3390/pathogens10020226 PMID: 33670728
64. Toledo A, Coleman J L, Kuhlow C J, Crowley J T, Benach J L. The enolase of Borrelia burgdorferi is a plasminogen receptor released in outer membrane vesicles. Infect Immun. 2012; 80(1):359-68. https://doi.org/10.1128/IAI.05836-11 PMID: 22083700
65. Nogueira S V, Smith A A, Qin J H, Pal U. A surface enolase participates in Borrelia burgdorferi-plasmino-gen interaction and contributes to pathogen survival within feeding ticks. Infect Immun. 2012; 80(1):82-90. https://doi.org/10.1128/IAI.05671-11 PMID: 22025510
66. Floden A M, Watt J A, Brissette C A. Borrelia burgdorferi enolase is a surface-exposed plasminogen bind-ing protein. PLoS One. 2011; 6(11):e27502. https://doi.org/10.1371/journal.pone.0027502 PMID: 22087329
67. Bendtsen J D, Kiemer L, Fausbøll A, Brunak S. Non-classical protein secretion in bacteria. BMC Micro-biol. 2005; 5:58. https://doi.org/10.1186/1471-2180-5-58 PMID: 16212653
68. Notti R Q, Stebbins C E. The structure and function of type III secretion systems. Microbiol Spectr. 2016; 4(1):10. https://doi.org/10.1128/microbiolspec.VMBF-0004-2015 PMID: 26999392
69. Young G M, Schmiel D H, Miller V L. A new pathway for the secretion of virulence factors by bacteria: The flagellar export apparatus functions as a protein-secretion system. Proc Natl Acad Sci USA. 1999; 96 (11):6456-646. https://doi.org/10.1073/pnas.96.11.6456 PMID: 10339609
70. Young B M, Young G M. YplA is exported by the Ysc, Ysa, and flagellar type III secretion systems of Yer-sinia enterocolitica. J Bacteriol. 2002; 184(5):1324-34. https://doi.org/10.1128/jb.184.5.1324-1334. 2002 PMID: 11844761
71. Konkel M E, Klena J D, Rivera-Amill V, Monteville M R, Biswas D, Raphael B, et al. Secretion of virulence proteins from Campylobacterjejuni is dependent on a functional flagellar export apparatus. J Bacteriol. 2004; 186(11):3296-303. https://doi.org/10.1128/J B.186.11.3296-3303.2004 PMID: 15150214
72. Barrero-Tobon A M, Hendrixson D R. Flagellar biosynthesis exerts temporal regulation of secretion of specific Campylobacter jejuni colonization and virulence determinants. Mol Microbiol. 2014; 93(5):957—
74. https://doi.org/10.1111/mmi.12711 PMID: 25041103
73. Abby S S, Rocha E P. The non-flagellar type III secretion system evolved from the bacterial flagellum and diversified into host-cell adapted systems. PLoS Genet. 2012; 8(9):e1002983. https://doi.org/10. 1371/journal.pgen.1002983 PMID: 23028376
74. Feltcher M E, Sullivan J T, Braunstein M. Protein export systems of Mycobacterium tuberculosis: novel targets for drug development? Future Microbiol. 2010; 5(10):1581-97. https://doi.org/10.2217/fmb.10. 112 PMID: 21073315
75. Kudva R, Denks K, Kuhn P, Vogt A, Müller M, Koch H-G. Protein translocation across the inner mem-brane of Gram-negative bacteria: the Sec and Tat dependent protein transport pathways. Research in microbiology. 2013; 164(6):505-34. https://doi.org/10.1016/j.resmic.2013.03.016 PMID: 23567322
76. Posey J E, Gherardini F C. Lack of a role for iron in the Lyme disease pathogen. Science. 2000; 288 (5471):1651-3. https://doi.org/10.1126/science.288.5471.1651 PMID: 10834845
77. Samuels D S, Radolf J D. Who is the BosR around here anyway? Mol Microbiol. 2009; 74(6):1295-9. https://doi.org/10.1111/j.1365-2958.2009.06971.x PMID: 19943896
78. Samuels D S. Gene regulation in Borrelia burgdorferi. Annu Rev Microbiol. 2011; 65:479-99. https://doi.org/10.1146/annurev.micro.112408.134040 PMID: 21801026
79. Oosting M, Buffen K, van der Meer J W, Netea M G, Joosten L A. Innate immunity networks during infec-tion with Borrelia burgdorferi. Crit Rev Microbiol. 2016; 42(2):233-44. https://doi.org/10.3109/1040841X.2014.929563 PMID: 24963691
80. Benach J L, Fleit H B, Habicht G S, Coleman J L, Bosler E M, Lane B P. Interactions of phagocytes with the Lyme disease spirochete: role of the Fc receptor. J Infect Dis. 1984; 150(4):497-507. https://doi.org/10.1093/infdis/150.4.497 PMID: 6386995
81. Menten-Dedoyart C, Faccinetto C, Golovchenko M, Dupiereux I, Van Lerberghe P B, Dubois S, et al. Neutrophil extracellular traps entrap and kill Borrelia burgdorferi sensu stricto spirochetes and are not affected by Ixodes ricinus tick saliva. J Immunol. 2012; 189(11):5393-401. https://doi.org/10.4049/jimmunol.1103771 PMID: 23109724
82. Nair S, Finkel S E. Dps protects cells against multiple stresses during stationary phase. J Bacteriol. 2004; 186(13):4192-8. https://doi.org/10.1128/J B.186.13.4192-4198.2004 PMID: 15205421
83. Wang G, Hong Y, Olczak A, Maier S E, Maier R J. Dual Roles of Helicobacter pylori NapA in inducing and combating oxidative stress. Infect Immun. 2006; 74(12):6839-46. https://doi.org/10.1128/IAI. 00991-06 PMID: 17030577
84. Grant R A, Filman D J, Finkel S E, Kolter R, Hogle J M. The crystal structure of Dps, a ferritin homolog that binds and protects DNA. Nat Struct Biol. 1998; 5(4):294-303. https://doi.org/10.1038/nsb0498-294 PMID: 9546221
85. Karas V O, Westerlaken I, Meyer A S. The DNA-Binding Protein from Starved Cells (Dps) Utilizes Dual Functions To Defend Cells against Multiple Stresses. J Bacteriol. 2015; 197(19):3206-15. https://doi. org/10.1128/J B.00475-15 PMID: 26216848
86. Purser J E, Norris S J. Correlation between plasmid content and infectivity in Borrelia burgdorferi. Proc Natl Acad Sci USA. 2000; 97(25):13865-70. https://doi.org/10.1073/pnas.97.25.13865 PMID: 11106398
87. Samuels D S. Electrotransformation of the spirochete Borrelia burgdorferi. Methods Mol Biol. 1995; 47:253-9. https://doi.org/10.1385/0-89603-310-4:253 PMID: 7550741
88. Caimano M J, Eggers C H, Gonzalez C A, Radolf J D. Alternate sigma factor RpoS is required for the in vivo-specific repression of Borrelia burgdorferi plasmid 1p54-borne ospA and 1p6.6 genes. J Bacteriol. 2005; 187(22):7845-52. https://doi.org/10.1128/J B.187.22.7845-7852.2005 PMID: 16267308
89. Paintdakhi A, Parry B, Campos M, Irnov I, Elf J, Surovtsev I, et al. Oufti: an integrated software package for high-accuracy, high-throughput quantitative microscopy analysis. Mol Microbiol. 2016; 99(4):767-77. https://doi.org/10.1111/mmi.13264 PMID: 26538279
90. Mastronarde D N. Automated electron microscope tomography using robust prediction of specimen movements. J Struct Biol. 2005; 152(1):36-51. https://doi.org/10.1016/j.jsb.2005.07.007 PMID: 16182563
91. Kremer J R, Mastronarde D N, McIntosh J R. Computer visualization of three-dimensional image data using IMOD. J Struct Biol. 1996; 116(1):71-6. https://doi.org/10.1006/jsbi.1996.0013 PMID: 8742726
92. Zuckert W R. Laboratory maintenance of Borrelia burgdorferi. Curr Protoc Microbiol. 2007; Chapter 12: Unit 12C 1. https://doi.org/10.1002/9780471729259.mc12c01s4 PMID: 18770608
93. Tinevez J Y, Perry N, Schindelin J, Hoopes G M, Reynolds G D, Laplantine E, et al. TrackMate: An open and extensible platform for single-particle tracking. Methods. 2017; 115:80-90. https://doi.org/10.1016/j. ymeth.2016.09.016 PMID: 27713081

### Example 2

### Introduction

Peptidoglycan is a potent immune response stimulant. Fortunately, this structural polymer is only found in bacteria, and there are no homologues to it in the human system, making it a perfect target for destruction by the innate immune system. The molecular patterns found in PG can be extracellularly sensed via outer membrane TLRs or intracellularly via NLRs and in some cases can be sensed vice versa via these same receptors. Either way, the downstream effectors upregulated by the presence of PG, trigger the translocation of transcription factors into the nucleus and act to upregulate pro-inflammatory responses.

The two most broad categories of bacterial PG are the Gram-negative and Gram-positive bacteria that differ in the cross-linking amino acid in the third position be it mDAP or lysine respectively. The model Gram-negative organism is E. coli, has the basic MurNAc-Ala-Glu-mDAP PG structure(1,2). Similarly, Streptococcus mutans has the typical Gram-positive PG structure MurNAc-Ala-Glu-Lys(3). Structural aberrations to the PG layer are common in bacteria as there has been an evolutionary drive to alter PG structures to enhance structural integrity and avoid immune system stimulation. For example, the Gram-positive Bacillus subtilis does not incorporate lysine in its PG structure, but instead has amidated-DAP, a deviation that has proven to dampen immune signaling by the NLRs in vitro (4). Staphylococcus aureus is known for two PG structural changes 1) the 0-acetylation of the MurNAc sugar, which makes this PG structure resistant to lysozyme (5) and; 2) the presence of a penta-glycine bridge that acts to provide additional structure support to the cell (6).

Perhaps among the most unique and understudied alterations to PG though, is that of B. burgdorferi. This structure is different from other more common bacteria in that it does not simply acetylate or amidate preexisting structural moieties, rather has incorporated a completely new amino acid, L-Ornithine (7). The Ornithine alteration is seen in some spirochetes as well as bacteria from the Thermus, Eubacteria, and Deinococcus genera (2, 8, 9). Based on the distinctive properties of this PG structure, the hypothesis of this chapter is that B. burgdorferi PG produces a unique response when compared to human immune cells stimulated with other bacterial PG, and that the PG of B. burgdorferi is responsible for the pathogenesis of Lyme arthritis. Additionally, we sought to answer the question of whether or not B. burgdorferi PG alone could recapitulate the immune response seen when immune cells were stimulated with live

B. burgdorferi. Insights into the immunological implications of the unique PG structure found in B. burgdorferi, compared to those of more well-known bacterium, are presented here using RNA sequencing analysis.

### Methodology

### Bacterial Strains

The Wild type strains of E. coli (K-12 strain MG1655), B. subtilis strain (168), S. mutans (Clarke), S. aureus strain (USA300) and a clone of the B. burgdorferi strain B31 were utilized in these experiments. The B. burgdorferi was cultured at 37° C. under 5% CO₂ in BSK II medium supplemented with 6% rabbit serum. S. mutans was grown in BIT medium at 37° C. All other bacteria were grown in Luria-Bertaini broth at 37° C. to an OD₆₀₀ of 0.6-0.7.

### Peptidoglycan Purification

Peptidoglycan from Gram-negative (E. coli and B. burgdorferi) bacterium was purified as described previously (7) and was isolated from 1 L of culture. Gram-positive (S. mutans, S. aureus, and B. subtilis) were isolated according to the same protocol with the following additional steps. Prior to solubilization in 5% SDS, the Gram-positive pellets were aliquoted out into 1 mL homogenizing tubes containing small glass beads and shaken in a Mini Bead Beater at 60% power for 2 minutes per tube. The tubes were reconsolidated, and the pellet was then solubilized in 5% SDS. The pellets were subjected to the same ultracentrifugation processing while being washed with deionized water at each step. Following chymotrypsin treatment, the samples were resuspended in 800 μL water and split into two microcentrifuge tubes each holding 400 μL of sample. One hundred microliters of 5M HCl was added and the tubes were placed on a rotating rack at 4° C. for 48 hours. The Gram-positive samples were then treated in the same manner as the Gram-negative samples following the protocol listed above. Peptidoglycan concentration was determined by dry weight following lyophilization.

### PBMC Culturing

Three, cryopreserved pooled samples of peripheral blood mononuclear cells (PBMCs) at a concentration of 1×10⁸ cells/mL from Zen-Bio were thawed and pooled into one batch of pooled cells in Lymphocyte culture media (Zen-Bio). These cells were plated in 12 well plates at a concentration of 2×10⁶ cells/mL and rested for 12 hours prior to stimulation. Following stimulation for 12 or 72 hours with 50 μg/mL PG from each strain, live B. burgdorferi strain A3 centrifugation at 800×g for 5 min at 15° C. The plates along with the supernatants that were collected, and aliquoted were all kept at −80° C.

### RNA Isolation

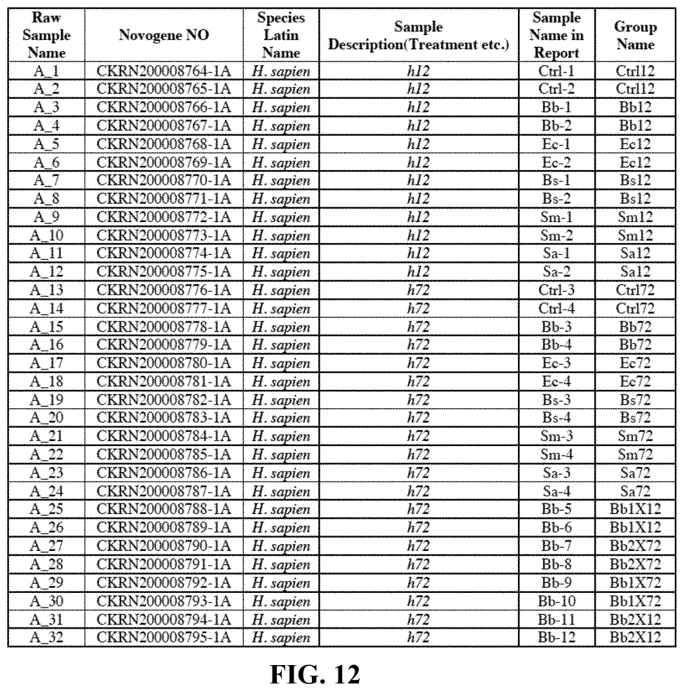

Twelve well plates containing PBMCs were thawed on ice for 5 minutes and equilibrated to room temperature for another 2-3 minutes prior to lysis with TRI Reagent (Sigma). RNA was precipitated with isopropyl alcohol and the pellets were washed twice with 75% ethanol before DNAse I (Zymo Research) treatment. The digested samples were further purified using the RNA Miniprep Plus Kit from Zymo Research. RNA quality and quantity were confirmed using Nanodrop. The samples were then frozen at −80° C. before being shipped on dry ice to NovoGene Co., Ltd (https://en.novogene.com/) for analysis. Names of samples provided to Novogene Co., Ltd are listed in FIG. 12.

### RNA Sequencing

Note: All RNA sequencing and the subsequent data analysis was performed by Novogene Co., Ltd. The following are summaries of the company's protocols used and can be found here (10). A complimentary DNA (cDNA) library was constructed and sequencing was done using Illumina, sequencing by synthesis, technology. Novogene performed quality control error rate and GC content distribution on the raw reads. Prior to gene expression analysis, raw reads with the adaptor sequences P5 and P7 (https://en.novogene.com/) were removed as well as reads with uncertain nucleotide sequences greater than 10% or Q-scores of >50%. Quality control checked reads were then aligned to the human genome (USCS hg 38) as the reference genome using the ‘Spliced Transcripts Alignment to a Reference’ (STAR v2.6.1) software. Read count is correlated to the counts produced by HTSeq v0.6.1 and these two values were used to calculate the Fragments per kilobase of transcript per million base pairs sequenced (FPKM) to normalize transcript levels.

### Differential Gene Expression Analysis

All experimental and control samples groups were composed of two biological replicates. Differential expression analysis was performed using the DESeq2 R Package (v2_1.6.3).

### Cluster Analysis

Samples were clustered using the assigned FPKM value and heatmaps were generated using self-organization mapping (SOM) using default parameters in R.

### Functional Analysis

Gene ontology enrichment analysis (GO), Kyoto Encyclopedia of Genes and Genomes (KEGG) Human Disease Ontology (DO) and Reactome analysis of differentially expressed genes was performed using clusterProfiler R (v2.4.3) package. Terms with a padj<0.05 are considered significantly enriched when compared to controls. Gene ontology, KEGG, coExpression, and clustering heatmap figures were all produced as part of the analysis performed by Novogene Co., Ltd.

### Results

### Purified PG Tnduces an Acute Pro-inflammatory Transcriptomic Profile in PBMCs

Purified PG from B. burgdorferi (PG^{Bb}), E. coli (PG^{Ec}), B. subtilis (PG_{Bs}), S. mutans (PG^{Sm}), and S. aureus (PG^{Sa}), were used to stimulate human PBMCs over the course of 12 and 72 hours. Additionally, PBMCs were stimulated with two concentrations of live B. burgdorferi, 500 cells/mL, for a ratio of B. burgdorferi to PBMCs of 1:4000 or 5000 cells/mL for a ratio of B. burgdorferi to PBMCs of 1:400. After stimulation with the PG of interest or live B. burgdorferi (referred to as live Borrelia), RNA was extracted from the samples, concentration and purity were verified via Nanodrop, and sent to Novogene Co., Ltd. for RNA sequencing analysis.

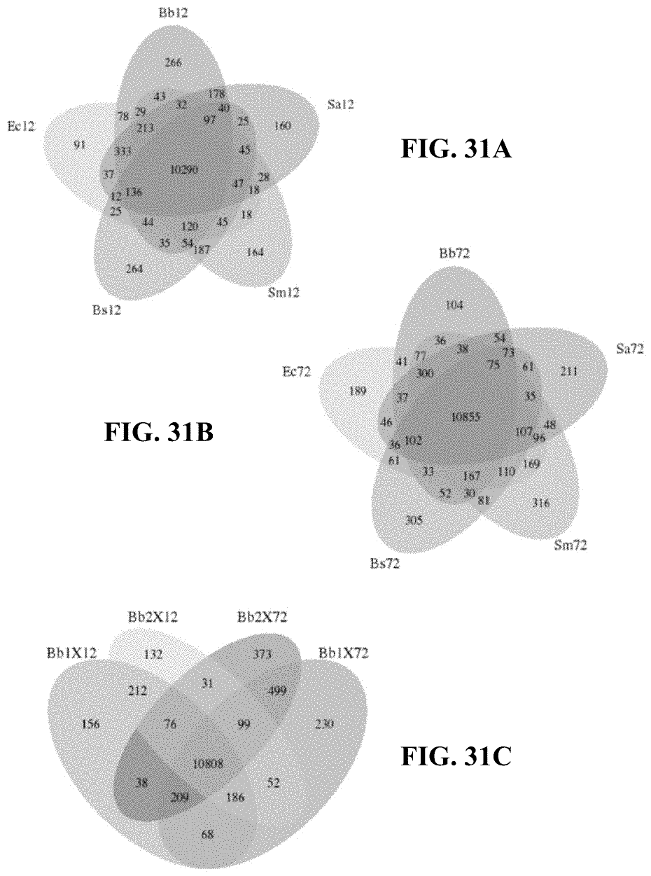

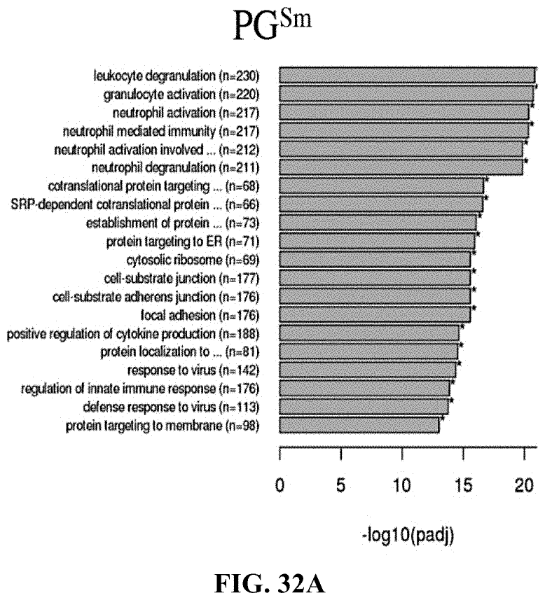

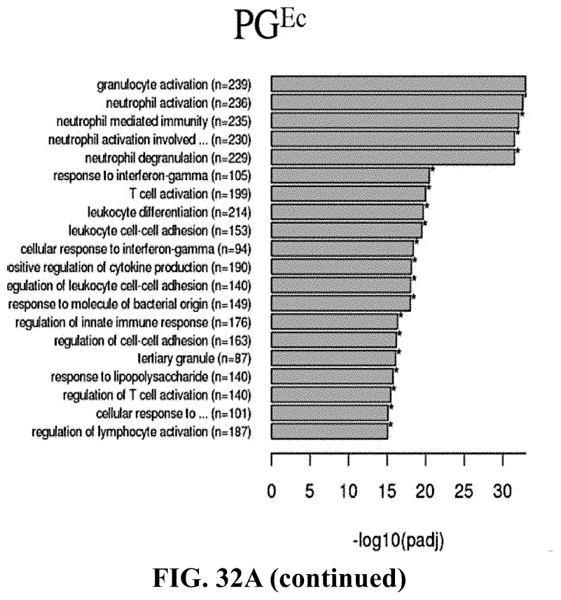

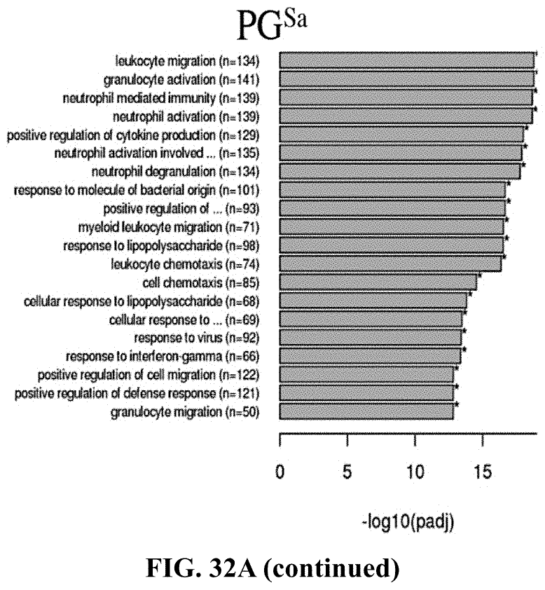

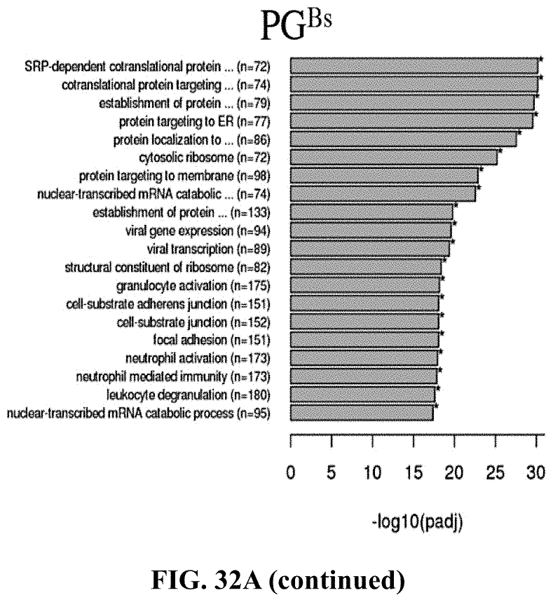

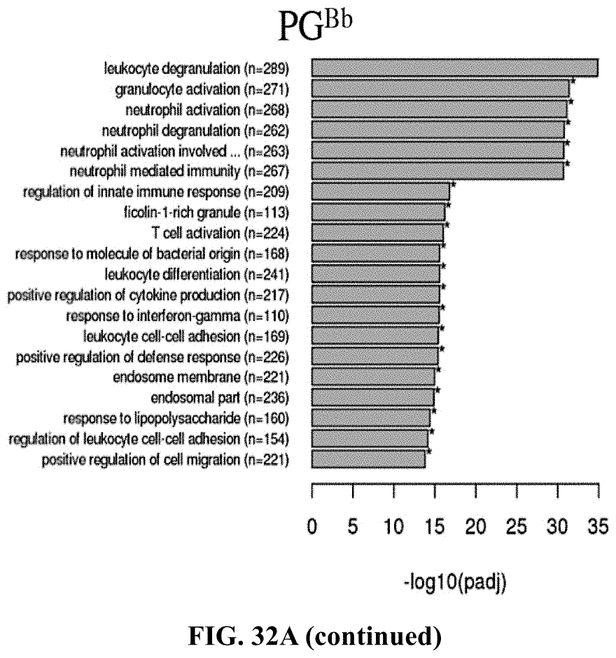

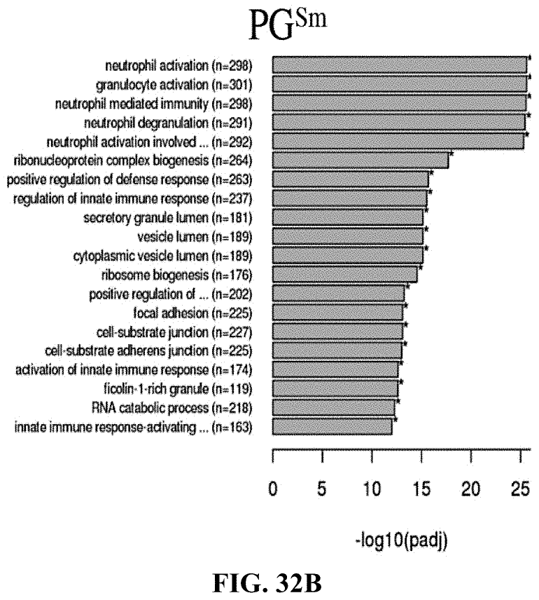

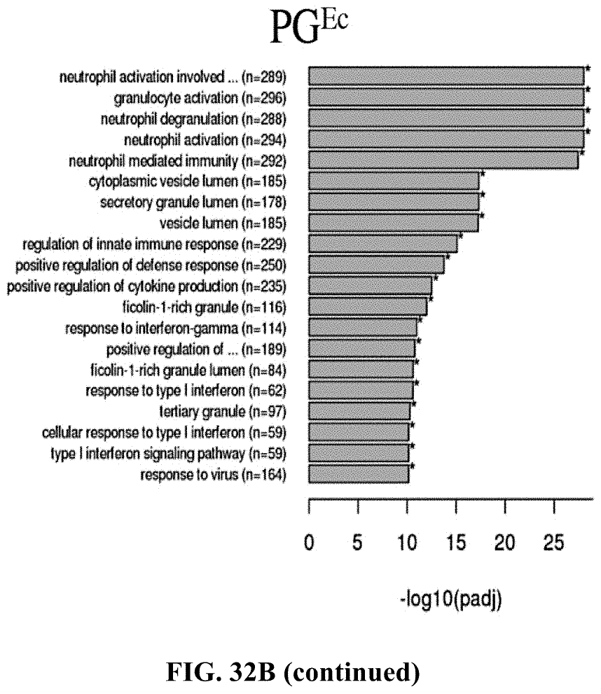

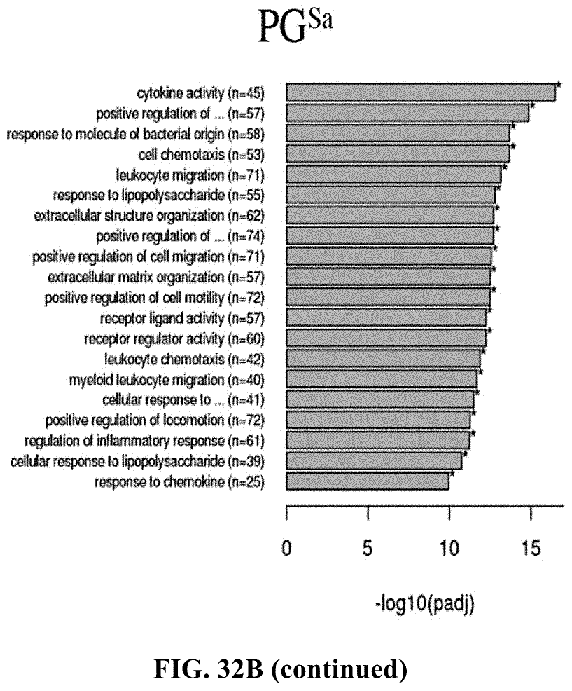

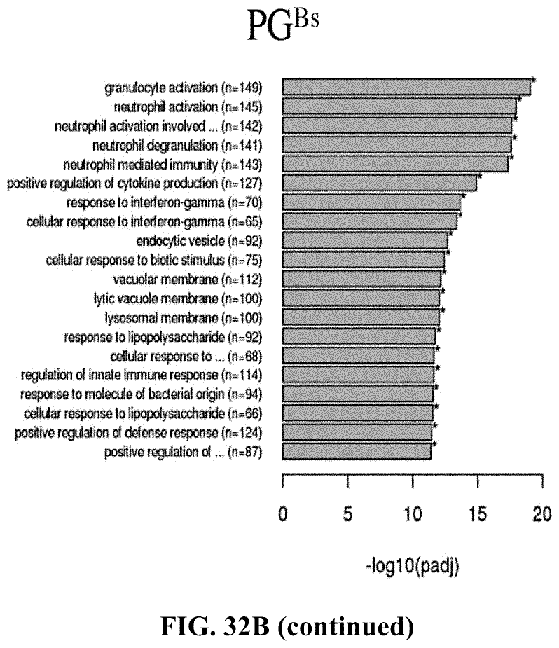

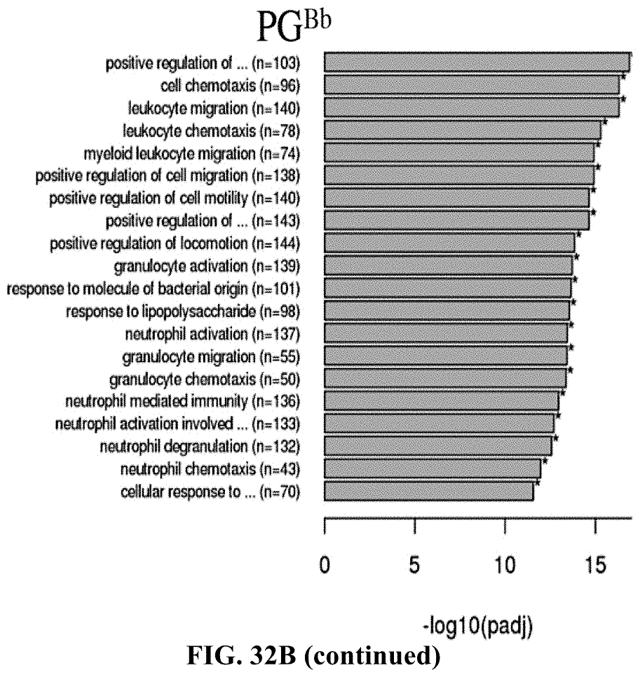

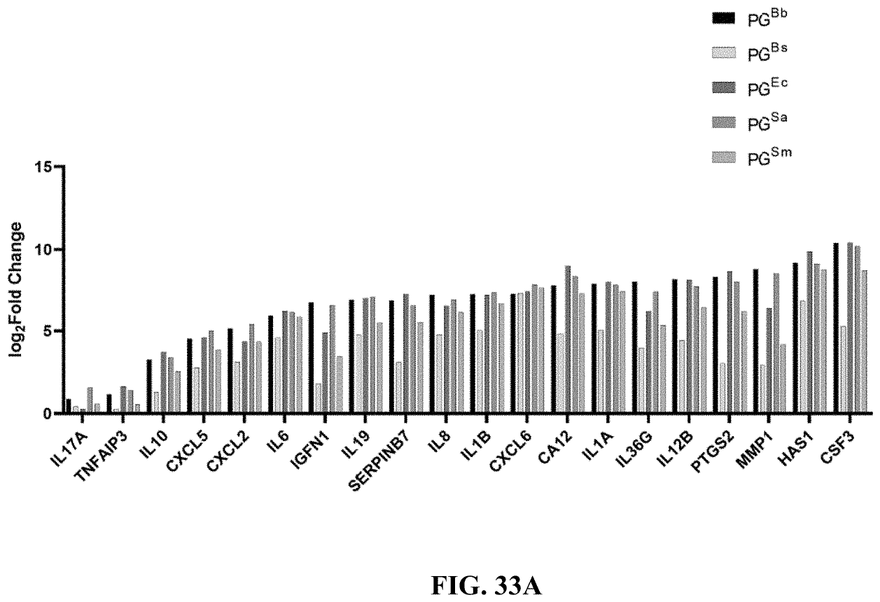

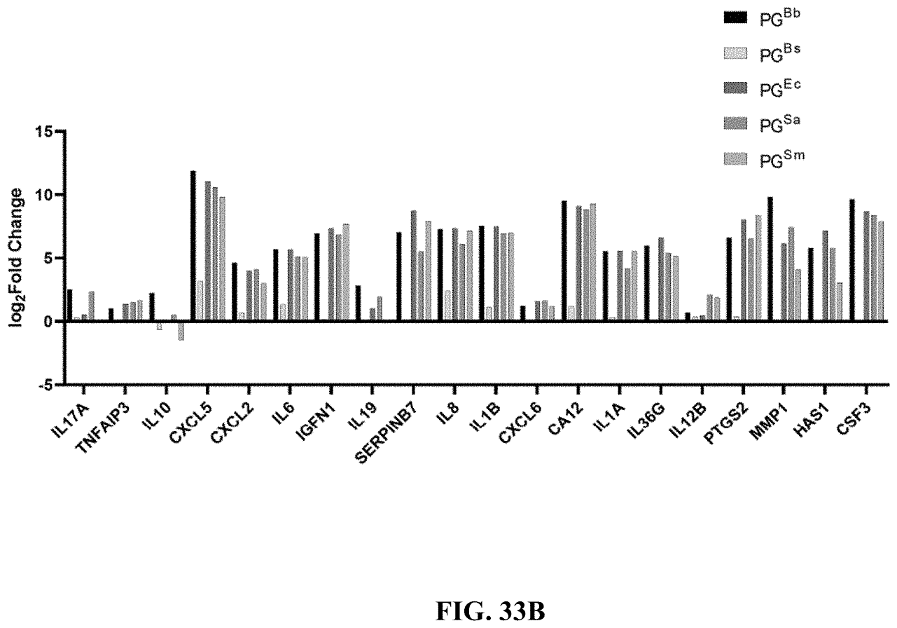

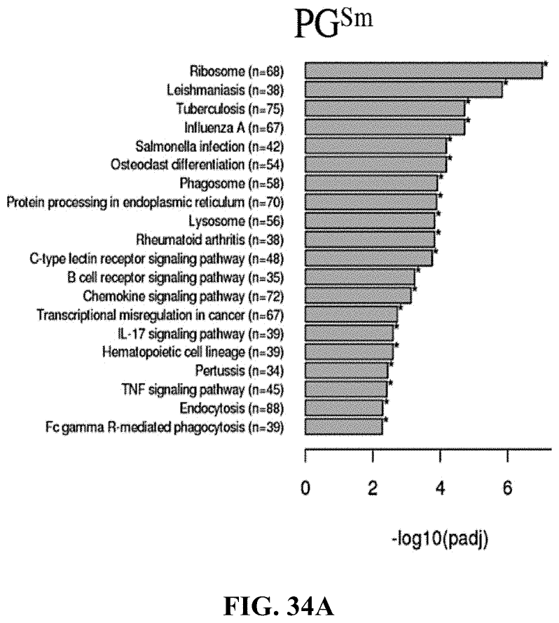

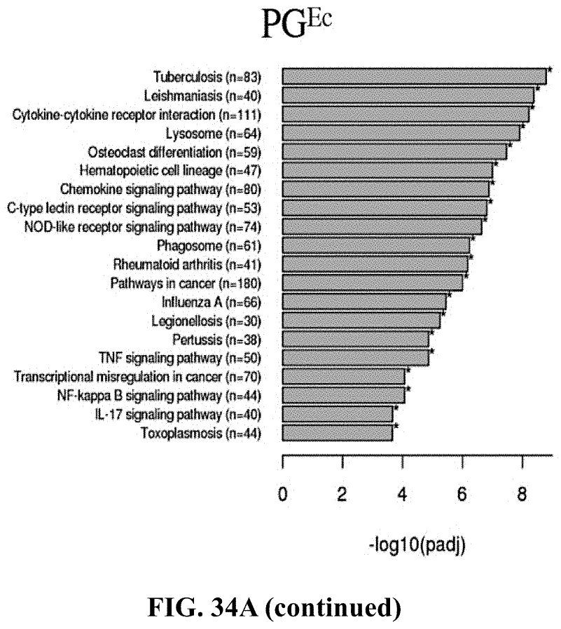

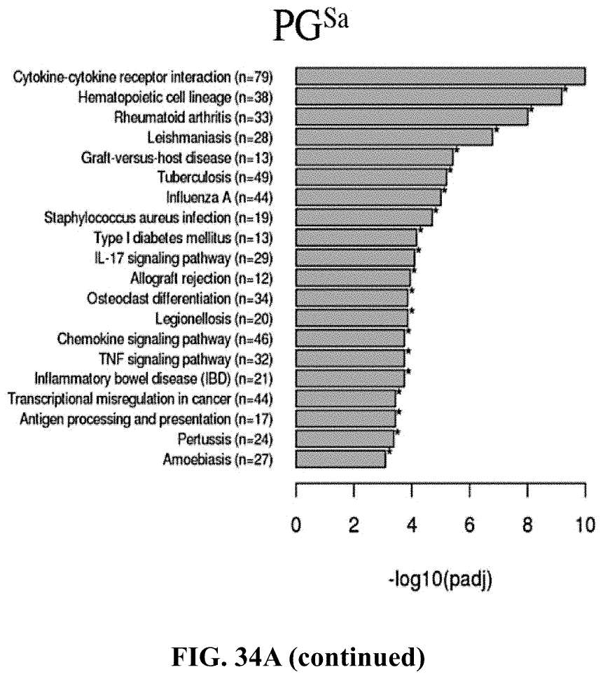

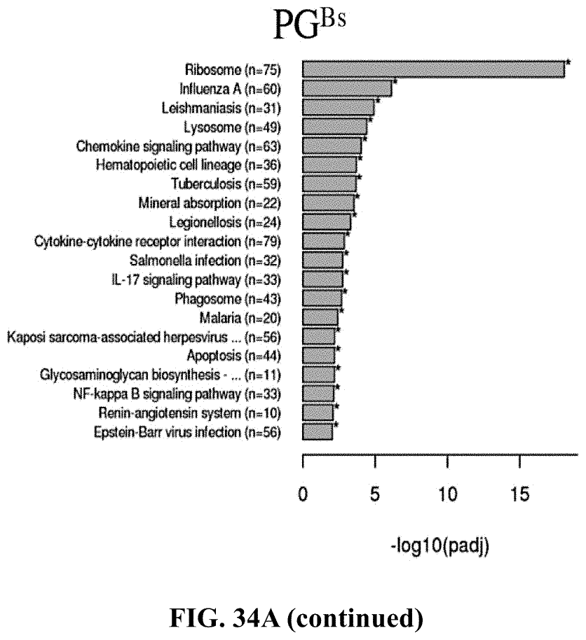

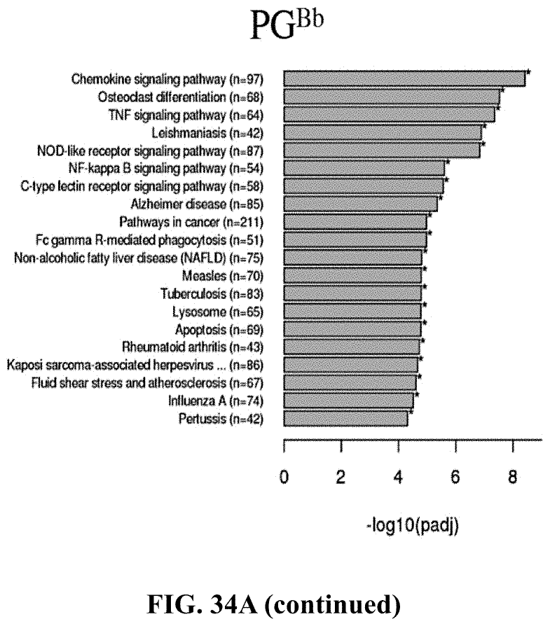

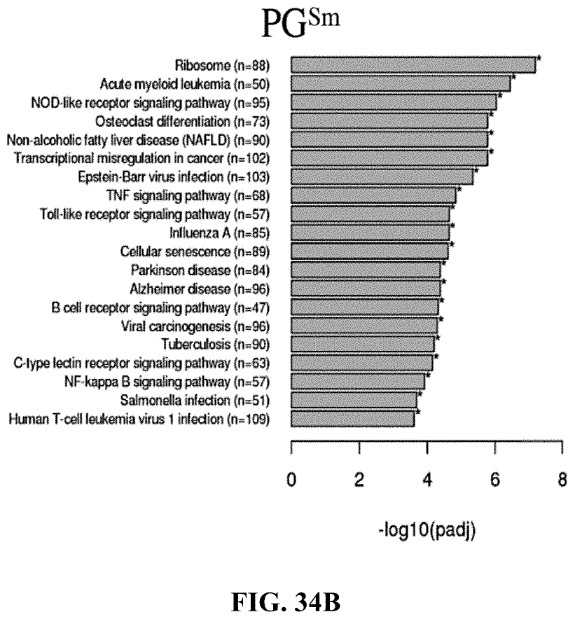

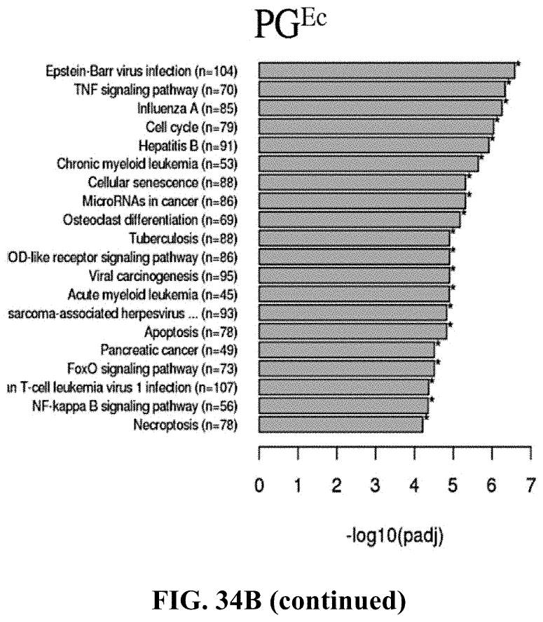

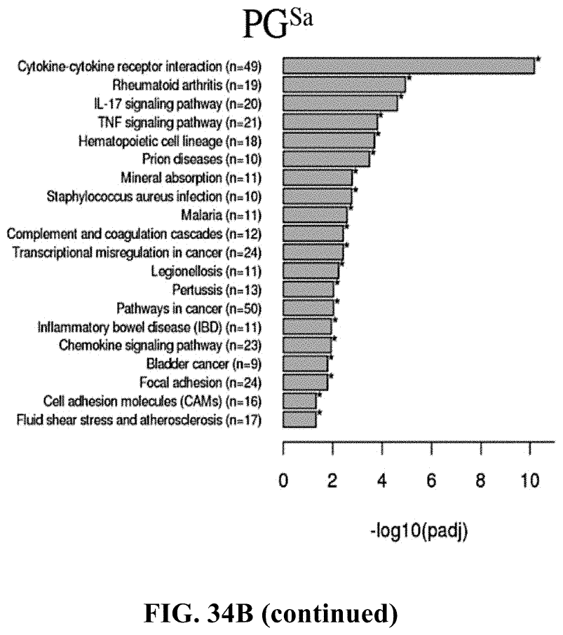

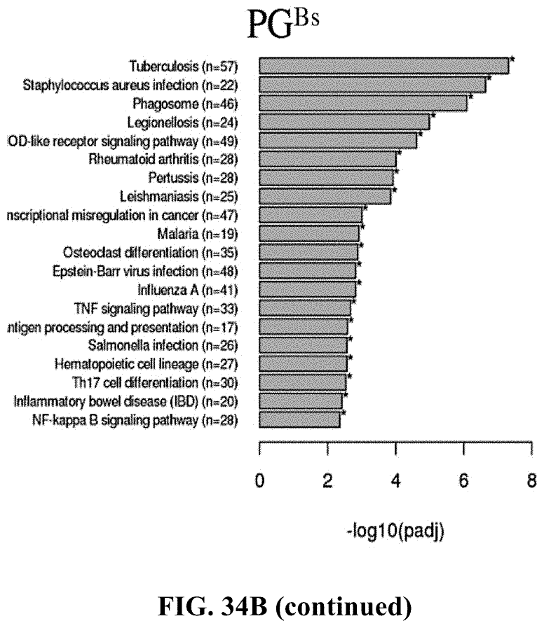

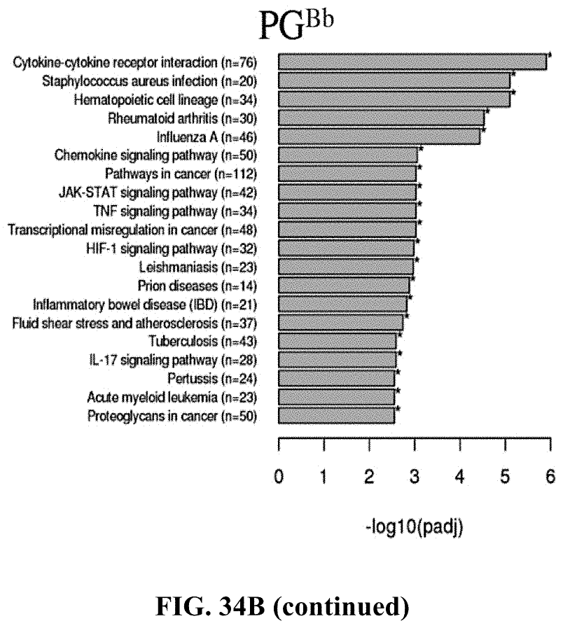

Among all the PGs, there were a total of 10,290 and 10,855 co-expressed genes observed at 12 and 72 hours respectively (FIGS. 31A and 31B). Tn contrast, when PBMCs were stimulated with live Borrelia there were 10,808 co-expressed genes shared between the two concentrations used (FIG. 31C). Peptidoglycan from all samples tested at 12 hours, with the exception of B. subtilis, caused a statistically significant change in genes involved in leukocyte degranulation, granulocyte activation, neutrophil activation and regulation of the innate immune response according to the Gene Ontology (GO) Enrichment analysis (FIG. 32A). Among some of the most prevalent differentially expressed genes represented in the study were TNFATP3, TL1A/B, TL6, CXCL2, CXCL12, CXCL5, and TL8, all of which were seen at 12 hours when compared to controls (FIG. 33A) (FIGS. 13 and 14). Kyoto Encyclopedia of Genes and Genomes analysis (KEGG) shows that after acute stimulation with all PGs used, with the exception of B. subtilis, there is strong correlation with innate immune signaling pathways including involvement of the TNF signaling pathway, NOD-like receptor signaling pathway, and the NF-kB signaling pathway (FIG. 34A).

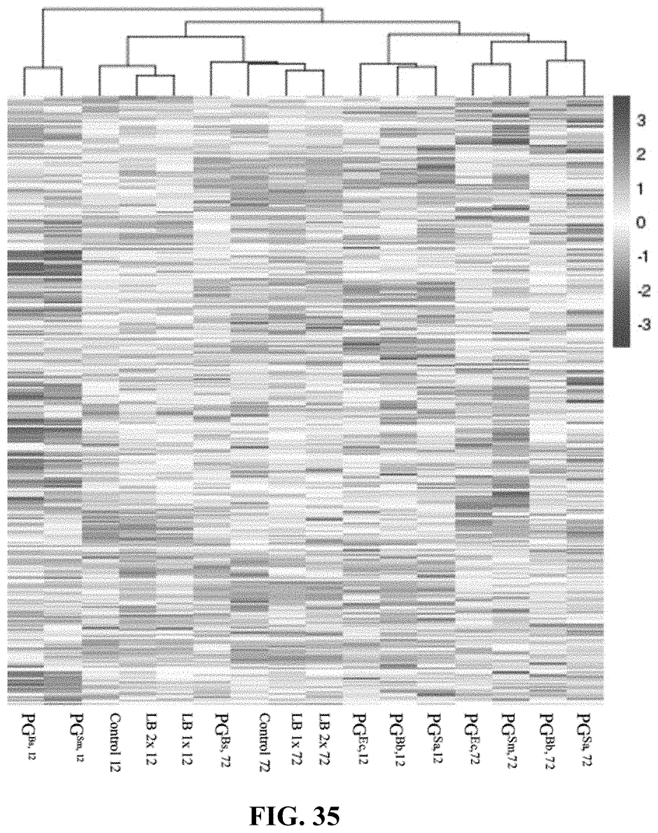

Cluster analysis of the differentially expressed genes among all experimental samples shows that there is a 1) distinct difference between PBMCs stimulated for 12 hours versus 72 hours; 2) clustering of coexpressed genes shared between both PG^{Bb}/PG^{Ec} and PG^{Bb}/PG^{Sa}; and 3) PG^{Bs} clusters more closely to controls than to samples stimulated with other PGs (FIG. 35). It is interesting to note that PBMCs stimulated with PG^{Bb}/PG^{Sa} cluster together at both 12 and 72 hours considering the respective differences in PG structure. A similar pattern is shared between PG^{Sm}/PG^{Ec} 72 hours post stimulation. The larger clusters separate the experimental groups by time exposed to the stimulant perhaps suggesting an equally important role for both PG structure and length of time exposed to the PG.

B. burgdorferi PG Initiates a Unique Immune Response in PBMCs

There were a few notable differences in the acute response seen in PBMCs stimulated with PG^{Bb} when compared to the other experimental samples. First, PBMCs stimulated with PG^{Bb} for 12 hours had the highest number of uniquely expressed genes (266) in comparison to the other PG stimuli used (FIG. 31A, FIG. 12)). Interestingly, the number of uniquely expressed genes dropped over 2-fold when cells stimulated with PG^{Bb} for 12 hours were compared to those stimulated for 72 hours (FIG. 31B) (FIG. 12). Secondly, according to the GO analysis, PBMCs stimulated with PG^{Bb} for 12 hours regulate gene products that function in pathways involving the use of ficolin-1-rich granules, a response seen only in PG^{Bb} with a padj value of 1×10⁻¹⁵ (FIG. 32A). Notable among these genes were TNFAIP6, TGFB1 and FGL2 which function in hyaluronan binding, cell growth/homeostasis, and prothrombinase activity respectively (11). Ficolin-1 or M-ficolin is produced by neutrophils and monocytes when stimulated with Gram-negative bacterial products. Of note, M-ficolin has been reported to correlate with neutrophil count in rheumatoid arthritis (RA) patients and SNPs in the M-ficolin gene has been connected to susceptibility to RA (12). Connections between these findings present interesting avenues to be explored in future studies.

Dysregulation in the balance between osteoblast (bone forming cells) and osteoclasts (bone resorbing cells) is a prominent feature in rheumatic diseases including RA and osteoarthritis (13). Among the most highly expressed genes in the in the PBMCs stimulated with PG^{Bb} for 12 hours when compared to controls were CSF3, HAS1, and MMP1, all of which function to maintain homeostasis within the cells of the bone marrow, joints, and extracellular matrix respectively (FIG. 33A) (FIGS. 13-14) (11). Note that among the other PGs tested these genes also had similar log 2 fold changes when compared to controls. After 12 hours of stimulation, PG^{Bb} KEGG pathway analysis in PBMCs showed the second highest correlation with the osteoclast differentiation pathway with 68 differentially expressed genes compared to control and a padj of 2.98×10⁻⁸ (FIG. 34A, far right). Note that this pathway as also seen in the PG^{Ec} and PG^{Sm} at 12 hours post stimulation. Interestingly, this pathway is not seen in PBMCs stimulated with PG^{Bb} for 72 hours although the pathway still remains at the same time point with PG^{Ec} and PG^{Sm} (FIG. 5B). Following the trend, the genes mentioned above are also less prominently expressed among the PGs tested at 72 hours (FIG. 33B).

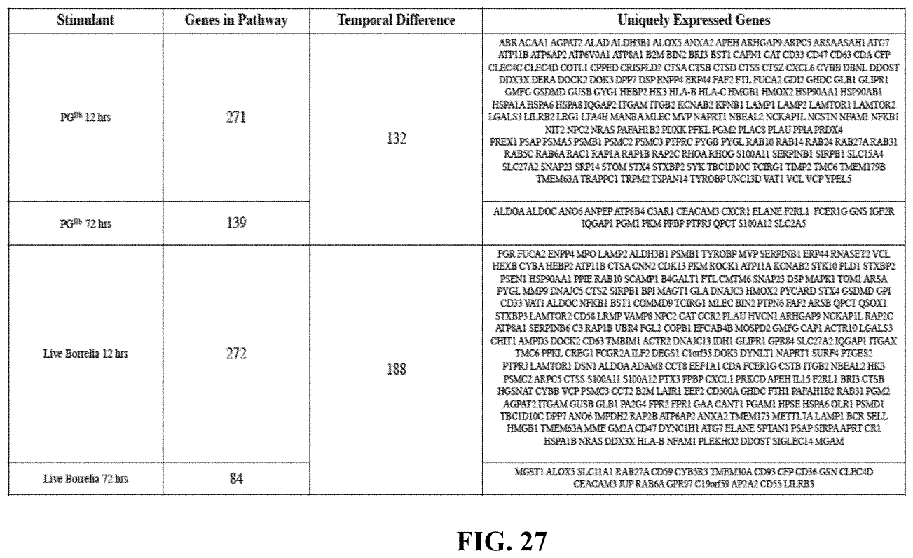

There is a prominent shift in the response seen from PBMCs when considering the 12- and 72-hour stimulations with PG^{Bb}. The GO enrichment analysis for all other PG samples (with the exception of S. aureus) shows the continued involvement of genes correlated to neutrophil degranulation, granulocyte activation, and regulation of innate immunity at 72 hours post stimulation (FIG. 32B). Although genes involved in these pathways are still represented, PG^{Bb} and PG^{Sa} both show a shift in GO association toward pathways involving cell migration, leukocyte migration, and cell motility (FIG. 32B). For example, the GO pathway “Granulocyte activation” resulting from PBMCs stimulated with PG^{Bb} for 12 hours is composed of 271 genes, this same pathway at 72 hours post stimulation is composed of only 139 genes. Among the genes no longer represented at 72 hours are the potent neutrophil activator CXCL6 (FIG. 27) (11); for comparison, there are no GO pathways representing chemotaxis in PBMCs stimulated with PG^{Bb} for 12 hours (FIGS. 32A and 32B). At 12 hours post stimulation with PG^{Bb} the KEGG pathways enriched are the innate TNF, NLR, NF-kB, and complement mediated pathways (FIG. 34A). In contrast, in PBMCS stimulated with PG^{Bb} for 72 hours the only innate pathway remaining is the TNF signaling pathway whereas all other PGs maintain at least 2 of the innate pathways seen at 12 post stimulation (FIG. 34B). Reactome Enrichment analysis supports a shift in innate pathways pertaining to neutrophil degranulation in PBMCS stimulated with PG^{Bb} at 72 hours when compared to those at 12 hours with a change in padj from 6.89×10⁻²⁴ to 3.35×10⁻¹² and that this change in PBMCs is unique to PG^{Bb} (FIGS. 21 and 26).

B. burgdorferi PG Recapitulates the Overarching Response from PBMCs Stimulated with Live Borrelia

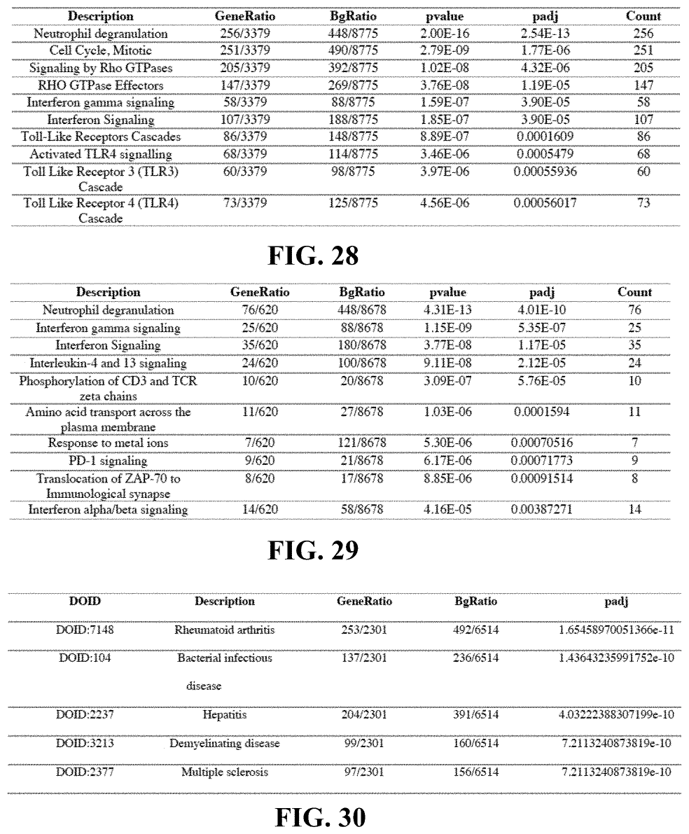

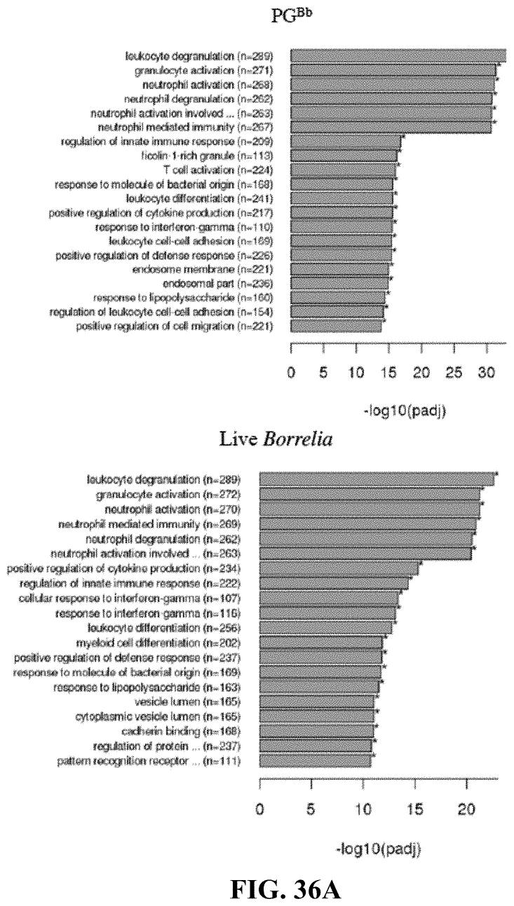

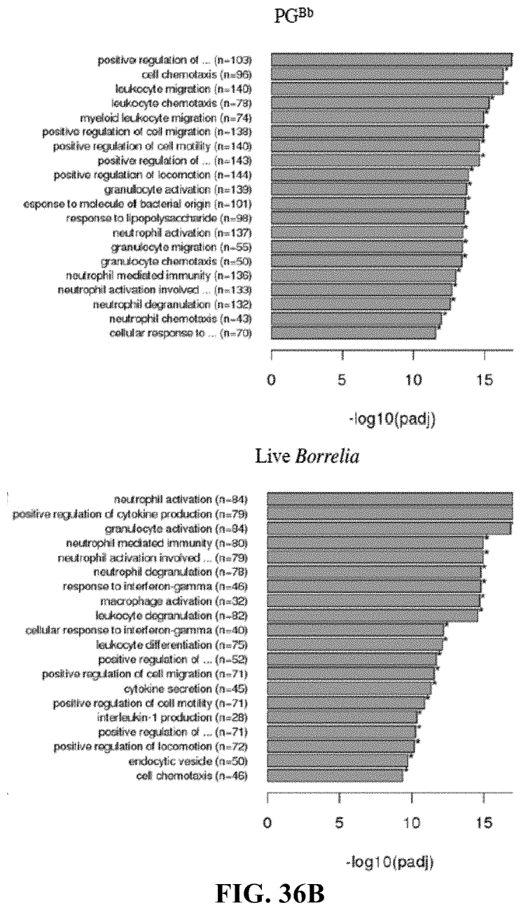

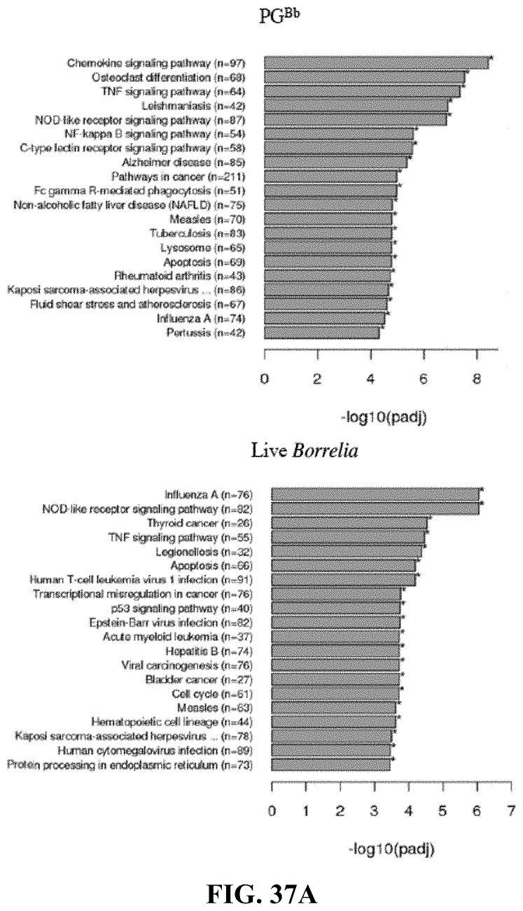

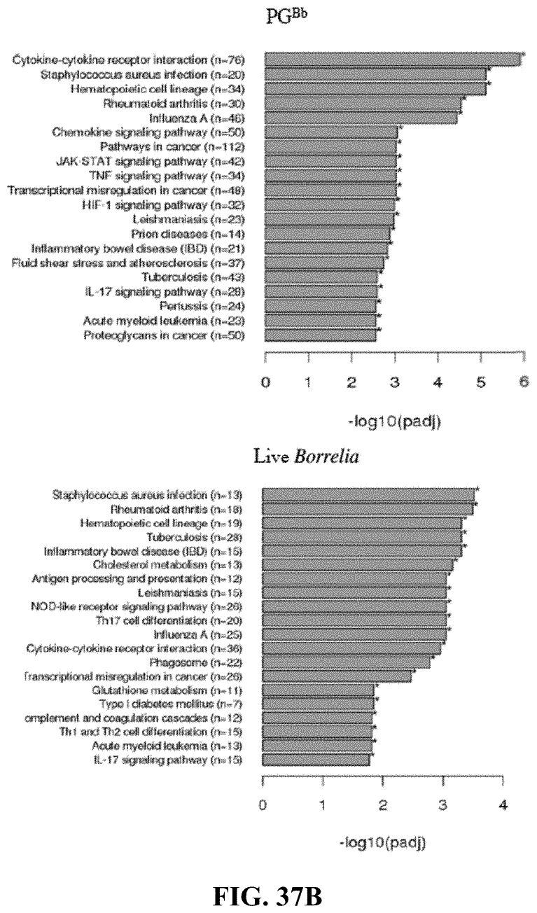

Peptidoglycan is released from B. burgdorferi under normal growth conditions (7). Thus, muropeptides may be free to interact with cellular receptors causing downstream effectors to activate. Although there is a near 2-fold decrease at 12 hours and a 2-fold increase at 72 hours of differentially expressed genes between PG^{Bb} and live Borrelia (FIG. 31C) (FIG. 27), GO, KEGG and Reactome enrichment analysis show similarities in the genes and pathways invoked by both stimuli. Similar to the GO results from PG^{Bb} at 12 hours, the PBMCs stimulated with live Borrelia for 12 hours also upregulated genes responsible for neutrophil degranulation, granulocyte activation, and regulation of innate immunity (FIG. 36A). The aforementioned observed shift in gene function to cell chemotaxis and motility is also seen in the PBMCs stimulated with live Borrelia at 72 hours according to GO analysis (FIG. 36B). Innate signaling pathways TNF, NLR, NF-kB and JAK-STAT KEGG pathways are seen in both the 12- and 72-hour samples when stimulated with either PG^{Bb} or live Borrelia (FIGS. 37A and 37B). Considering the findings in the next chapter, it is also interesting that both stimulants also cause an upregulation in genes involved in the IL-17 pathway 72 hours post stimulation according to KEGG analysis (FIG. 37B). The temporal change in genes corresponding to neutrophil degranulation seen in PG^{Bb} is also seen in the Reactome enrichment analysis of live cells stimulated with live Borrelia (FIGS. 28-29).

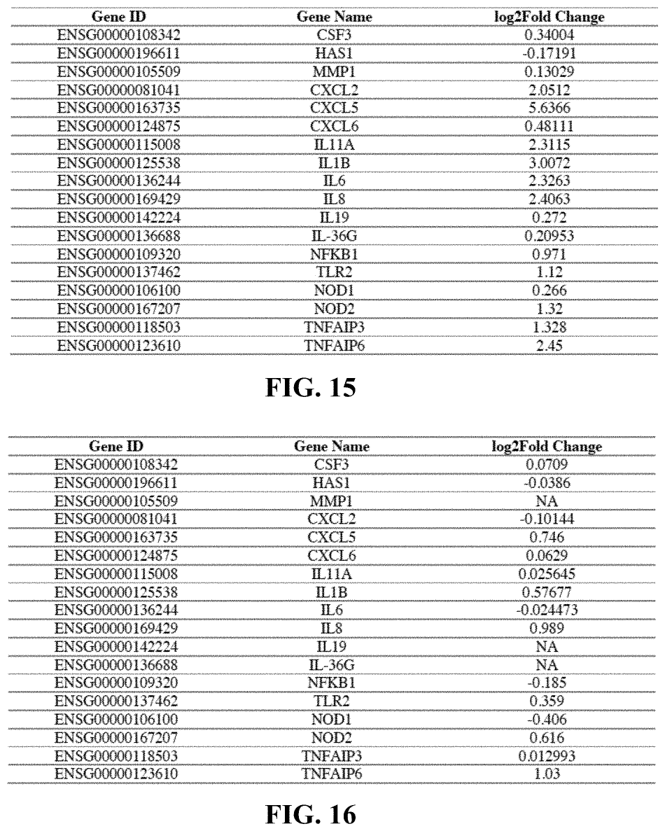

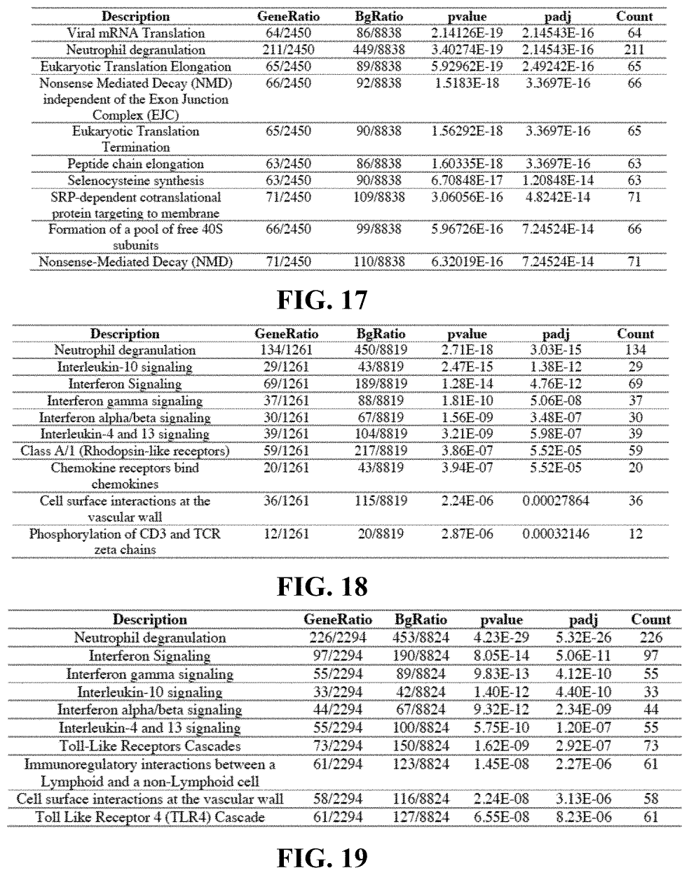

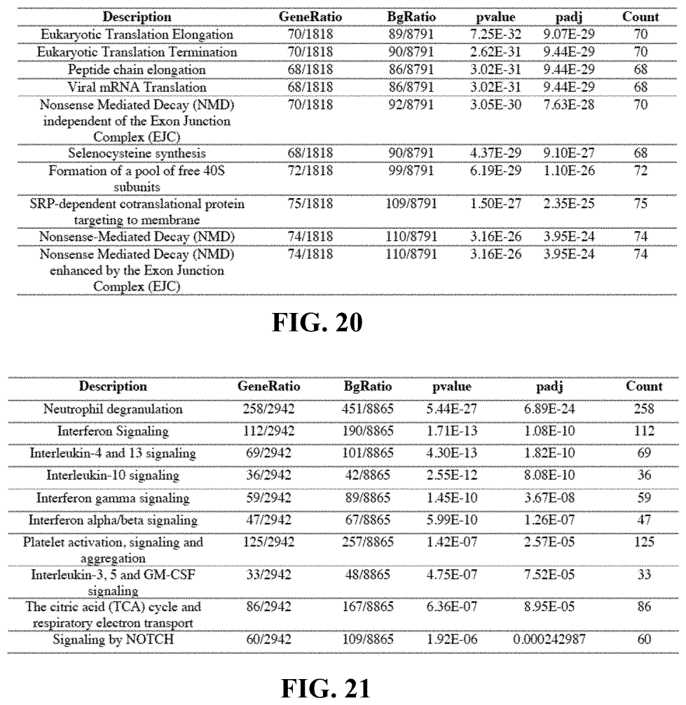

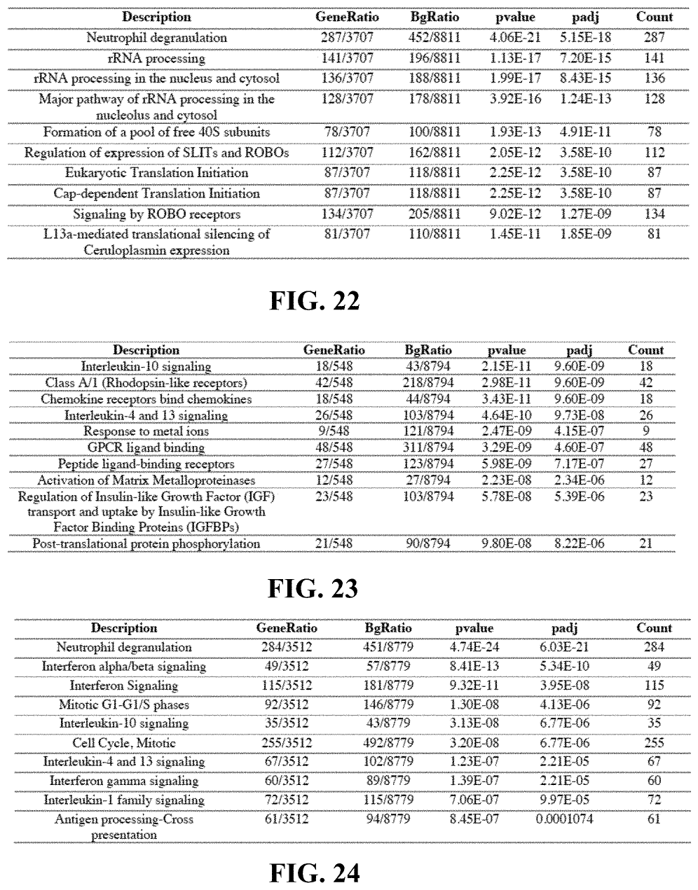

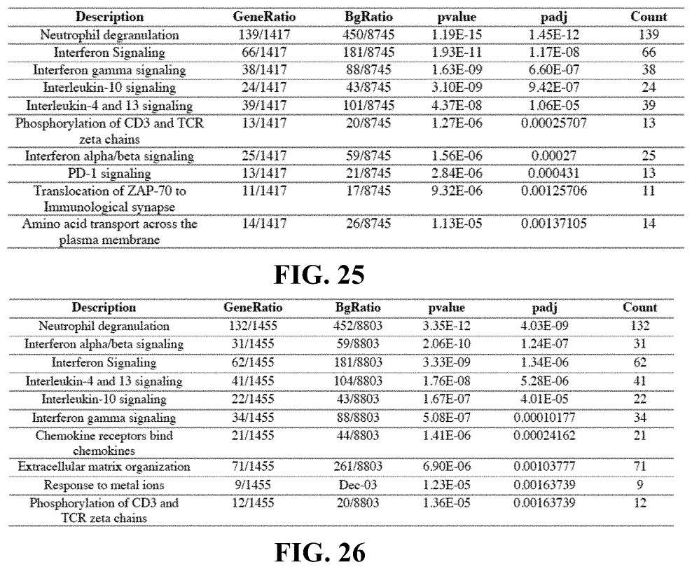

Additional results are shown in FIGS. 15-25.

### Discussion

The studies outlined here provide supporting evidence for the notion that PG^{Bb} is a potent and unique immune system modulator. Using bulk RNA sequencing technology, we found that PG^{Bb} elicits a similar acute pro-inflammatory transcriptomic profile in PBMCs and that this response is virtually universal among the other PGs used in this study. Although there are many similarities in the acute profile seen in PBMCs stimulated with PG, there are unique differences seen only in those stimulated with PG^{Bb}. These differences carry over into the response seen 72 hours post stimulation with PG^{Bb} and correlate well with the responses seen when PBMCs are stimulated with live Borrelia instead of PG.

The PGs used in this study virtually all upregulated acute pro-inflammatory genes and together best correlated to the disease genotype associated with RA according to Disease Ontology (DO) analysis (FIG. 30). This is an interesting finding considering there are a handful of studies correlating bacterial products such as PG with acute synovitis and arthritic phenotypes (7, 5, 9 14-16). These data confirm a strong role for previous bacterial infections with the onset of rheumatic disease, and correlate with the symptoms seen in Lyme arthritis. It is important to note that in these studies there is no evidence of an active bacterial infection at the time of the arthritic onset. Instead, it is hypothesized that the bacterial antigens are the source of the inflammation. More specifically, there is often no evidence of a bacterial infection in RA patients, but there is supporting evidence for small quantities of bacterial DNA in their joints (14). In addition, the condition known as reactive arthritis (ReA) in which patients present with arthralgia, conjunctivitis and urinary tract inflammation, is also often a result of a previously resolved bacterial infection (17). Lyme arthritis shares many features with these other rheumatic diseases in that in about 10% of cases, Lyme arthritis may occur after a patient has undergone proper antibiotic treatment (18). The genes that play a role in the pathways involved in the DO enrichment analysis are seen when PBMCs are stimulated with both PG^{Bb} and live Borrelia. Most notable among these genes are the three with the highest fold change when compared to controls, CSF3, HAS1 and MMP1 (FIG. 33A). Colony stimulating factor 3 (CSF3) specifically acts on bone marrow cells to produce granulocytes, such as neutrophils, perhaps to areas of acute inflammation caused by the exposure to PG (11). Hyaluronan synthase 1 (HAS1) is involved in the joint lubrication process and lastly, matrix metallopeptidase 1 (MMP1) belongs to a family of peptidases that remodel the extracellular matrix and collagen, interestingly both are also implicated in rheumatic disease (11). The genes listed here in FIG. 33A namely CXCL6, CXCL5, CXL2, IL6 and MMP1 were all recently reported as upregulated in fibroblast-like synoviocytes (FLS) stimulated with B. burgdorferi (19), supporting the notion that PG alone is able to recapitulate the transcriptomic phenotype seen when stimulating with B. burgdorferi.

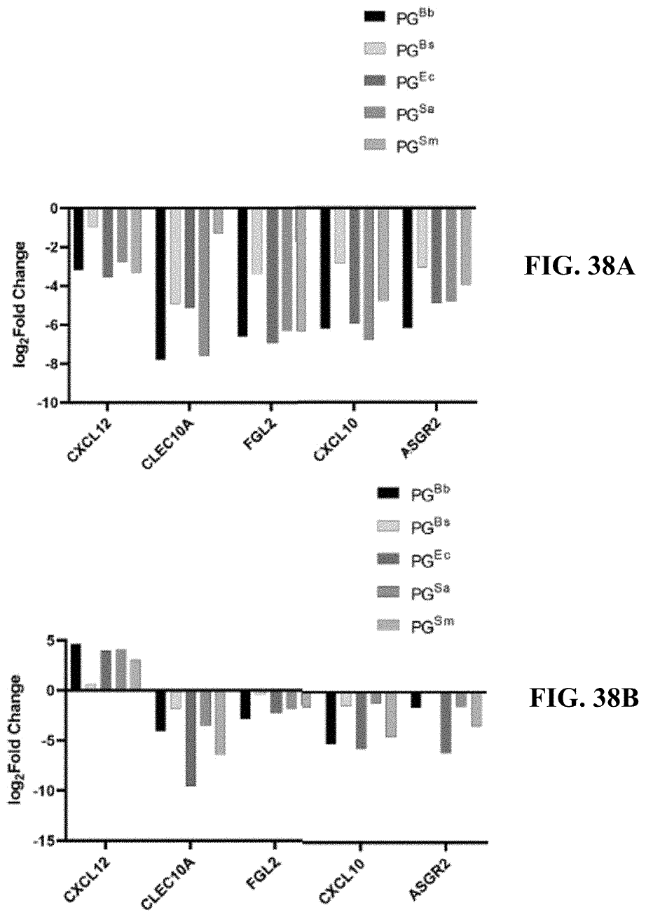

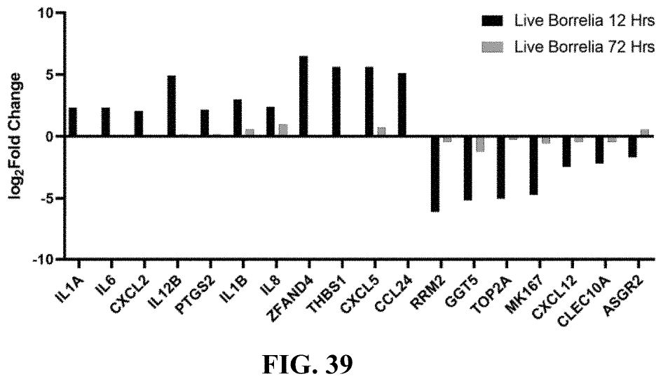

The shift in transcriptomic profile expressed by PBMCs stimulated with PG^{Bb} at 12 hours versus 72 hours presents an interesting finding. It is perhaps not coincidental that much like other bacterial PGs tested here, there is an acute onset of pro-inflammatory genes when PG^{Bb} is the stimulus. The intracellular activation of NOD-2 is dependent on the presence of MDP, a ubiquitous structure in PG. Interestingly, there is only a notable change in NOD2 expression in PBMCs that have been stimulated with live Borrelia at 12 hours, perhaps indicating that PG alone requires the aid of portions of the B. burgdorferi cell/cell envelope to bind to the intracellular NOD receptor (FIG. 15) or that there may be other inflammatory pathways involved here. At 72-hours post PG^{Bb}, and live Borrelia, stimulation, there is a decrease in pro-inflammatory innate pathways and an increase in leukocyte migration, cell motility, and chemotaxis as seen in both the GO and KEGG analysis results (FIG. 32B and FIG. 34B). These pathways are supported by the downregulation of the genes encoding the pro-inflammatory molecules IL-1A, IL-6, and CXCL6 at 72-hours post PG^{Bb} (FIG. 33B). Peptidoglycan from B. burgdorferi also appears to suppress the activation of chemotactic cytokines CXCL12 and CXCL10 from PBMCs stimulated at 12 hours, whereas this response is lessened at 72 hours (FIGS. 38A and 38B). In a similar manner, stimulation with live Borrelia at 12 hours results in an increase in ILIA and IL6 while CXCL12 is downregulated (FIG. 39). At 72 hours post stimulation with live Borrelia, the opposite is seen for these genes (FIG. 39). The dampening of the classical and robust immune response initially seen with PG^{Bb} could be an attributing factor to the pathogenesis of late stage Lyme symptoms. Perhaps the chronicity of low levels of inflammation driven by the presence of bacterial PG, as well as increased cell signaling for leukocyte infiltration, could correlate with Lyme arthritis which can occur anywhere from days to months after infection (20).

A recent study has shown that after antibiotic treatment, Lyme disease patients that develop Lyme arthritis have PG^{Bb} in their synovial fluid (23). Even after the eradication of the active infection, these bacterial components can still be detected using ELISAs to detect IgG antibodies to PG^{Bb} (7). The mouse model has been used to tie these findings together. When PG^{Bb} is injected in the tail vein of mice, they develop arthritis in their ankle joints 24 hours later (7). This systemic injection of purified PG indicates that the PG can travel through the body and eventually still end up in the synovial fluid, perhaps in a similar manner to the mechanism in the human system. The study above is supported by the RNA sequencing data presented here. The work presented in this chapter supports the hypothesis that PG^{Bb} is indeed capable of inducing inflammatory mediators that may play a role in the pathogenesis of Lyme arthritis.

### References for Example 2

1. Glauner B, Holtje J V, Schwarz U. The composition of the murein of Escherichia coli. J Biol Chem. 1988; 263(21): 10088-95.
2. Vollmer W, Blanot D, de Pedro M A. Peptidoglycan structure and architecture. FEMS Microbiol Rev. 2008; 32(2):149-67.
3. Slade H D, Slamp W C. Peptidoglycan composition and taxonomy of group D, E, and H streptococci, and Streptococcus mutans. J Bacteriol. 1972; 109(2):691-5.
4. Girardin S E, Travassos L H, Herve M, Blanot D, Boneca I G, Philpott D J, et al. Peptidoglycan molecular requirements allowing detection by Nod1 and Nod2. J Biol Chem. 2003; 278(43):41702-8.
5. Baranwal G, Mohammad M, Jarneborn A, Reddy B R, Golla A, Chakravarty S, et al. Impact of cell wall peptidoglycan O-acetylation on the pathogenesis of Staphylococcus aureus in septic arthritis. Int J Med Microbiol. 2017; 307(7):388-97.
6. Monteiro J M, Covas G, Rausch D, Filipe S R, Schneider T, Sahl H G, et al. The pentaglycine bridges of Staphylococcus aureus peptidoglycan are essential for cell integrity. Sci Rep. 2019; 9(1):5010.
7. Jutras B L, Lochhead R B, Kloos Z A, Biboy J, Strle K, Booth C J, et al. Borrelia burgdorferi peptidoglycan is a persistent antigen in patients with Lyme arthritis. Proceedings of the National Academy of Sciences. 2019:201904170.
8. Quintela J C, Garcia-del Portillo F, Pittenauer E, Allmaier G, de Pedro M A. Peptidoglycan fine structure of the radiotolerant bacterium Deinococcus radiodurans Sark. J Bacteriol. 1999; 181(1):334-7.
9. Zhang X, Rimpilainen M, Simelyte E, Toivanen P. What determines arthritogenicity of bacterial cell wall? A study on Eubacterium cell wall-induced arthritis. Rheumatology (Oxford). 2000; 39(3):274-82.
10. Novogene Co. L. Methods_MedTR [cited 2020]
11. database Gthg. 2016 [Available from: www.genecards.org.
12. Ammitzboll C G, Thiel S, Jensenius J C, Ellingsen T, Horslev-Petersen K, Hetland M L, et al. M-ficolin levels reflect disease activity and predict remission in early rheumatoid arthritis. Arthritis Rheum. 2013; 65(12):3045-50.
13. Corrado A, Maruotti N, Cantatore F P. Osteoblast Role in Rheumatic Diseases. Int J Mol Sci. 2017; 18(6).
14. van der Heij den I M, Wilbrink B, Tchetverikov I, Schrijver I A, Schouls L M, Hazenberg M P, et al. Presence of bacterial DNA and bacterial peptidoglycans in joints of patients with rheumatoid arthritis and other arthritides. Arthritis Rheum. 2000; 43(3):593-8.
15. Hannu T. Reactive arthritis. Best Pract Res Clin Rheumatol. 2011; 25(3):347-57.
16. Bockenstedt L K, Gonzalez D G, Haberman A M, Belperron A A. Spirochete antigens persist near cartilage after murine Lyme borreliosis therapy. J Clin Invest. 2012; 122(7):2652-60.
17. Selmi C, Gershwin M E. Diagnosis and classification of reactive arthritis. Autoimmun Rev. 2014; 13(4-5):546-9.
18. Steere A C. Lyme disease. N Engl J Med. 2001; 345(2):115-25.
19. Lochhead R B, Ordonez D, Arvikar S L, Aversa J M, Oh L S, Heyworth B, et al. Interferon-gamma production in Lyme arthritis synovial tissue promotes differentiation of fibroblast-like synoviocytes into immune effector cells. Cell Microbiol. 2019; 21(2):e12992.
20. Prevention CfiCa. Signs and Symptoms of Untreated Lyme iisease 2019 [Available from: https://www.cdc.gov/lymesigns_symptoms/index.html

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. A method of treating or preventing a Borrelia burgdorferi (B. burgdorferi) infection, a symptom thereof, or a disease, disorder or condition resulting therefrom in a subject in need thereof, the method comprising:
reducing or eliminating a B. burgdorferi peptidoglycan-associated protein (PAP), a function thereof, activity thereof, or any combination thereof in the subject in need thereof.

2. The method of claim 1, wherein the B. burgdorferi PAP is neutrophil attracting protein A (NapA).

3. The method of claim 2, wherein reducing or eliminating a B. burgdorferi peptidoglycan-associated protein (PAP), a function thereof, activity thereof, or any combination thereof in the subject in need thereof comprises administering a PAP inhibitor to the subject in need thereof, wherein the PAP inhibitor is optionally an antibody or fragment thereof capable of specifically binding the PAP or an enzyme capable of targeting, degrading, modifying, and/or otherwise inhibiting the PAP.

4. The method of claim 1, wherein the B. burgdorferi peptidoglycan-associated protein (PAP), a function thereof, activity thereof, or any combination thereof is reduced 1-5,000 fold.

5. The method of claim 1, wherein the disease, disorder, or condition resulting from the B. burgdorferi infection is inflammation, optionally neutrophil mediated-inflammation.

6. The method of claim 5, wherein the inflammation is intra-articular inflammation.

7. The method of claim 1, wherein the disease, disorder or condition resulting from B. burgdorferi infection is arthritis, optionally rheumatoid arthritis, carditis, encephalitis, paralysis, optionally neurological paralysis, a wound, or any combination thereof.

8. A method of diagnosing or prognosing a Borrelia burgdorferi (B. burgdorferi) infection, a symptom thereof, or a disease, disorder or condition resulting therefrom in a subject in need thereof, the method comprising:
detecting a B. burgdorferi peptidoglycan-associated protein (PAP) in a sample obtained from the subject in need thereof.

9. The method of claim 8, wherein the B. burgdorferi PAP is neutrophil attracting protein A (NapA).

10. The method of claim 8, wherein the sample comprises B. burgdorferi outer membrane vesicles.

11. The method of claim 10, wherein the PAP is present in the B. burgdorferi outer membrane vesicles.

12. The method of claim 8, further comprising detecting an amount of IL-17 in the sample, wherein an increase in IL-17 as compared to a suitable control indicates B. burgdorferi infection.

13. The method of claim 8, wherein detecting comprises exposing the sample or component(s) thereof to peripheral blood mononuclear cells in culture and measuring an amount IL-17 in the culture supernatant, whereby an increase in the amount of IL-17 as compared to a suitable control indicates the presence of a PAP.

14. The method of claim 13, wherein the PAP is neutrophil attracting protein A (NapA).

15. The method of claim 8, wherein the sample is a bodily fluid, optionally blood or fraction thereof or synovial fluid.

16. The method of claim 8, wherein detecting comprises mass-spectrometry, protein sequencing, an immunodetection method, or any combination thereof.

17. The method of claim 8, further comprising treating the Borrelia burgdorferi (B. burgdorferi) infection, a symptom thereof, or a disease, disorder or condition resulting therefrom in the subject in need thereof, wherein treating comprises reducing or eliminating a B. burgdorferi peptidoglycan-associated protein (PAP), a function thereof, activity thereof, or any combination thereof in the subject in need thereof.

18. The method of claim 17, wherein the PAP is neutrophil attracting protein A (NapA).

19. The method of claim 17, wherein treating comprises administering to the subject in need thereof comprises administering a PAP, optionally NapA, inhibitor to the subject in need thereof, optionally wherein the PAP inhibitor is an antibody or fragment thereof capable of specifically binding the PAP, optionally NapA protein, or an enzyme capable of targeting, degrading, modifying, and/or otherwise inhibiting the PAP, optionally the NapA protein.

20. The method of claim 17, wherein the B. burgdorferi peptidoglycan-associated protein (PAP), a function thereof, activity thereof, or any combination thereof is reduced 1-5,000 fold.
